SEQUENCE LISTING (13 sequences)

SEQ ID NO: 1 - protein, 340 aa, Herpes simplex virus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPG

SEQ ID NO: 2 - protein, 312 aa, Herpes simplex virus 2
MHAIAPRLLLLFVLSGLPGTRGGSGVPGPINPPNSDVVFPGGSPVAQYCYAYPRLDDPGPLGSADAGRQDLPRRVVRHEPLGRSFLTGGLVLLAPPVRGFGAPNATYAARVTYYRLTRACRQPILLRQYGGCRGGEPPSPKTCGSYTYTYQGGGPPTRYALVNASLLVPIWDRAAETFEYQIELGGELHVGLLWVEVGGEGPGPTAPPQAARAEGGPCVPPVPAGRPWRSVPPVWYSAPNPGFRGLRFRERCLPPQTPAAPSDLPRVAFAPQSLLVGITGRTFIRMARPTEDVGVLPPHWAPGALDDGPYAP

SEQ ID NO: 3 - protein, 625 aa, Macacine alphaherpesvirus
MRRLVPRLSLLAAVVCLCLGPAGAGPRPPAAPPDAAPAARDPAYCYAVPRLDDVGPAGPAPPVADVDRHPVVRHRNLSRSYSRRGLALLVPPIRRFRGTGGGAYAARVAYYRVDAGGCRRPIALRWYGGCRGGAPPSPSTCGRYSHTYHDGSPPMAHALVNASLLVPVVGSRAAAYDYEILVGDRLHVGRLTAAVAPGDPLSPPDRASRPDDRGRGGPAACRPVAPAVPLWRSIPAAWVVAANPLFDGPEGRRCVSPQDPAVPQDMAYVSAAPQSLLVGLSGYLFALGGRDREGEPPATEAPAPTAERFARARLARSLLSVTTEGTEASPASAPTVAPSAATEGASATSGSATAASAAVESATPTANGTAATPAPTTTPAPTTTPAPTTTPAPTTTPAPTTTPAPTTTPANGTAGAESDAGEGSKTTPATPTAAPSVSPAPAAPAANDTGAAGAGPVPSDATTPPGPTAAEENESSPPLPREGGGDSATGGASAAAGTEAPPADPFTPRLEALTPRPGAGGGAERGAGEEDGDDDDDEEGAGDGELPPAENPPPATGHAGPGARPDSHPYPPTPRRPSSRLPPPHLGPLTLRPPRPPTEPQAPSPSPTEPPHTPLFPFLTASPGL

SEQ ID NO: 4 - protein, 237 aa, Human cytomegalovirus
MNGWAGVCLITHCLNTRSRTYVALNMLAFARTPRGVPSCLFNKVWVSRYALVLILMVCASESSTSWAVTSNGLPNCSTVTRTAGQDAELHGPAPLSCNVTQWGRYENGSTPVLWCTLRGSRMRVSLGHRVAFGCSWKTFFIYNVSESSGGTYYQKGYNCTDKHITLSCFNLTVVPRAVQSTTTVMTPTLVTNSTFSVSLVALRLTTNSSAFGHAIYQRQQRVENGTLSKNITNLAFT

SEQ ID NO: 5 - protein, 192 aa, Human cytomegalovirus
MSKRLQVFPWITILFYTSKSQYWNYMTIPCTPTVGYGSHNISLHPLNNSLFQDDVFEWYIDKPMVTNKLCLYQSNERIKSNLDSPNIMWQCTDNRTLILMNLTTTYSRNYYFQSFKYLGQGVPKPNNLCYNVSVHFTHQTHCHTTTSSLYPPTSVHDSLEISQSFTSTNFTHTAVHYAAGNVEAQHDTATSH

SEQ ID NO: 6 - protein, 269 aa, Human cytomegalovirus
MRIEWACWLFGYFVSSVGSERSLSYRYHLESNSSANVVCNGNISVFVNGTLGVRYNITVGISSSLLIGHLTIQTLESWFTPWVQNKSYSKQPLSTTETLYNIDSENIHRVSQYFHTRWIKSLQENHTCDLTNSTPTYTYQANVNNTNYLTLTSSGWQDRLNYTAINSTHFNLTESNITSIHKYLNTTCIERLRNYTLEPVYTTAVPQNVTPEHAITTLYTTPPNAITIKDTTQSHTVQTPSFNDTHNVTEHTLNISYVLSQKTNNTTSP

SEQ ID NO: 7 - protein, 133 aa, Human cytomegalovirus
MKPLVMLICFGVILLQLGVTKVCQHNEVQLGNECCPPCGLGQRVTKVCTERTSVTCTPCPNGTYVSGLYNCTDCTQCNVTQVMIRNCTSTNNTVCAPKNHTYFSTPGVQHHKQRQQNHTAHITVKQGKSGRHT

SEQ ID NO: 8 - protein, 76 aa, Human cytomegalovirus
MYPRVMHAVCFLALGLISYVAVCAENTVTTNCLVKAENTHLTCKCNPNTTSNTNNGSKCHAMCKCRVTEPITMLGA

SEQ ID NO: 9 - protein, 528 aa, Varicella zoster virus
MKRIQINLILTIACIQLSTESQPTPVSITELYTSAATRKPDPAVAPTSAASRKPDPAVAPTSAASRKPDPAVAPTSAASRKPDPAVAPTSAATRKPDPAVAPTSAASRKPDPAVAPTSAATRKPDPAVAPTSAASRKPDPAANTQHSQPPFLYENIQCVHGGIQSIPYFHTFIMPCYMRLTTGQQAAFKQQQKTYEQYSLDPEGSNITRWKSLIRPDLHIEVWFTRHLIDPHRQLGNALIRMPDLPVMLYSNSADLNLINNPEIFTHAKENYVIPDVKTTSDFSVTILSMDATTEGTYIWRVVNTKTKNVISEHSITVTTYYRPNITVVGDPVLTGQTYAAYCNVSKYYPPHSVRVRWTSRFGNIGKNFITDAIQEYANGLFSYVSAVRIPQQKQMDYPPPAIQCNVLWIRDGVSNMKYSAVVTPDVYPFPNVSIGIIDGHIVCTAKCVPRGVVHFVWWVNDSPINHENSEITGVCDQNKRFVNMQSSCPTSELDGPITYSCHLDGYPKKFPPFSAVYTYDASTYATT

SEQ ID NO: 10 - protein, 786 aa, Varicella zoster virus
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPTRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNP

SEQ ID NO: 11 - protein, 272 aa, Varicella zoster virus
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPEN

SEQ ID NO: 12 - protein, 226 aa, Human herpesvirus 8
MSSLFISLPWVAFIWLALLGAVGGARVQGPMRGSAALTCAITPRADIVSVTWQKRQLPGPVNVATYSHSYGVVVQTQYRHKANITCPGLWNSTLVIHNLAVDDEGCYLCIFNSFGGRQVSCTACLEVTSPPTGHVQVNSTEDADTVTCLATGRPPPNVTWAAPWNNASSTQEQFTDSDGLTVAWRTVRLPRGDNTTPSEGICLITWGNESISIPASIQGPLAHDLP

SEQ ID NO: 13 - protein, 522 aa, Human herpesvirus 8
MAFLRQTLWILWTFTMVIGQDNEKCSQKTLIGYRLKMSRDGDIAVGETVELRCRSGYTTYARNITATCLQGGTWSEPTATCNKKSCPNPGEIQNGKVIFHGGQDALKYGANISYVCNEGYFLVGREYVRYCMIGASGQMAWSSSPPFCEKEKCHRPKIENGDFKPDKDYYEYNDAVHFECNEGYTLVGPHSIACAVNNTWTSNMPTCELAGCKFPSVTHGYPIQGFSLTYKHKQSVTFACNDGFVLRGSPTITCNVTEWDPPLPKCVLEDIDDPNNSNPGRLHPTPNEKPNGNVFQRSNYTEPPTKPEDTHTAATCDTNCEQPPKILPTSEGFNETTTSNTITKQLEDEKTTSQPNTHITSALTSMKAKGNFTNKTNNSTDLHIASTPTSQDDATPSIPSVQTPNYNTNAPTRTLTSLHIEEGPSNSTTSEKATASTLSHNSHKNDTGGIYTTLNKTTQLPSTNKPTNSQAKSSTKPRVETHNKTTSNPAISLTDSADVPQRPREPTLPPIFRPPASKNRYL

(19) United States
(12) Patent Application Publication
(10) Pub. No.: US 2024/0011017 A1
(43) Pub. Date: Jan. 11, 2024

(54) METHODS FOR MODULATING HOST CELL SURFACE INTERACTIONS WITH HERPESVIRUSES

(71) Applicant: Genentech, Inc., South San Francisco, CA (US)

(72) Inventors: Nadia MARTINEZ-MARTIN, San Francisco, CA (US); Bushra HUSAIN, Boston, MA (US)

(73) Assignee: Genentech, Inc., South San Francisco, CA (US)

(21) Appl. No.: 18/235,972

(22) Filed: Aug. 21, 2023

Related U.S. Application Data
(63) Continuation of application No. PCT/US2022/017301, filed on Feb. 22, 2022.
(60) Provisional application No. 63/236,917, filed on Aug. 25, 2021.
(60) Provisional application No. 63/152,295, filed on Feb. 22, 2021.

(51) Int. Cl. C12N 15/10; G01N 33/68

(52) U.S. Cl. CPC C12N 15/1055; C12N 15/1037; G01N 33/6845; G01N 2333/03; G01N 2500/02

(57) ABSTRACT

Provided herein are methods of treating or preventing herpesvirus infection comprising modulating interactions between herpesvirus surface proteins and plasma membrane-expressed host cell proteins, as well as methods of identifying modulators of such interactions.

### CROSS-REFERENCE TO RELATED APPLICATIONS

This application is a continuation of International Application No. PCT/US2022/017301, filed on Feb. 22, 2022, which claims benefit to U.S. Provisional Patent Application No. 63/152,295, filed on Feb. 22, 2021, and U.S. Provisional Patent Application No. 63/236,917, filed on Aug. 25, 2021, the entire contents of each of which are incorporated herein by reference in their entirety.

### SEQUENCE LISTING

The instant application contains a Sequence Listing which has been submitted electronically in XML format and is hereby incorporated by reference in its entirety. Said XML copy, created on Jul. 27, 2023, is named 50474-249004_Sequence_Listing_7_27_23 and is 16,604 bytes in size.

### FIELD OF THE INVENTION

Provided herein are methods of treating or preventing herpesvirus infection comprising modulating interactions between herpesvirus surface proteins and plasma membrane-expressed host cell proteins, as well as methods of identifying modulators of such interactions.

### BACKGROUND

Herpesviridae is a family of DNA viruses that cause infection (including latent, unapparent, and reactivating infection) and disease in animals, including humans.

Herpesviruses include herpes simplex virus 2 (HSV-2), which causes genital herpes and herpes simplex encephalitis; Macacine alphaherpesvirus (MCHV), which causes dangerous zoonotic infections in humans; Human cytomegalovirus (HCMV); varicella zoster virus (VZV), which causes chicken pox and shingles; and human herpesvirus 8, which causes Kaposi's sarcoma, HHV-associated multicentric Castleman's disease, primary effusion lymphoma, and KSHV inflammatory cytokine syndrome.

Much remains to be understood about the host cell receptors and cellular factors that mediate herpesvirus entry and initiation of infection, in part due to the lack of sensitive technologies for the study of membrane protein interactomes. This limited understanding has resulted in a dearth of therapeutically effective options for the treatment and prevention of herpesvirus infections and resulting diseases.

Thus, there is an unmet need for methods for identifying modulators of novel interactions between herpesviruses and host cells, as well as methods for treating or preventing herpesvirus infections using such modulators.

### SUMMARY OF THE INVENTION

In one aspect, the invention provides a method of identifying a modulator of the interaction between a protein of Table 1 and a protein of Table 2, the method comprising (a) providing a candidate modulator; (b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

In another aspect, the invention provides a method of identifying a modulator of a downstream activity of a protein of Table 1, the method comprising (a) providing a candidate modulator; (b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1.

In another aspect, the invention provides a method of identifying a modulator of a downstream activity of a protein of Table 2, the method comprising (a) providing a candidate modulator; (b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and (c) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

In some aspects, the increase or decrease in binding is at least 70%, as measured by a surface plasmon resonance (SPR) assay, a BLI assay, or an enzyme-linked immunosorbent assay (ELISA).

In some aspects, the modulator is an inhibitor of the downstream activity of the protein of Table 1 or Table 2. In other aspects, the modulator is an activator of the downstream activity of the protein of Table 1 or Table 2.

In some aspects, the change in the downstream activity is a decrease in the amount, strength, or duration of the downstream activity. In other aspects, the change in the downstream activity is an increase in the amount, strength, or duration of the downstream activity.

In some aspects, the downstream activity is infection of a cell by a member of the viral family Herpesviridae.

In some aspects, infection is decreased in the presence of the modulator. In some aspects, infection is decreased by at least 40%, as measured in a viral infection assay or a viral entry assay.

In some aspects, the modulator is a small molecule, an antibody or antigen-binding fragment thereof, a peptide, a mimic, an antisense oligonucleotide, or a small interfering RNA (siRNA). In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the antibody or antigen-binding fragment thereof binds the protein of Table 1.

In some aspects, the antibody or antigen-binding fragment thereof binds the protein of Table 2.

In another aspect, the invention provides a method of treating an individual having a herpes simplex virus 2 (HSV-2) infection comprising administering to the individual an effective amount of a CSPG5 antagonist, a PRRG2 antagonist, a UNC5D antagonist, or a PLB1 antagonist.

In another aspect, the invention provides a method of decreasing HSV-2 infection in an individual comprising administering to the individual an effective amount of a CSPG5 antagonist, a PRRG2 antagonist, a UNC5D antagonist, or a PLB1 antagonist.

In some aspects, (a) the CSPG5 antagonist results in a decrease in the binding of CSPG5 and the HSV-2 glycoprotein G (gG) protein relative to binding of the two proteins in the absence of the antagonist; (b) the PRRG2 antagonist results in a decrease in the binding of PRRG2 and the HSV-2 gG protein relative to binding of the two proteins in the absence of the antagonist; (c) the UNC5D antagonist results in a decrease in the binding of UNC5D and the HSV-2 gG protein relative to binding of the two proteins in the absence of the antagonist; or (d) the PLB1 antagonist results in a decrease in the binding of PLB1 and the HSV-2 gD protein relative to binding of the two proteins in the absence of the antagonist.

In some aspects, the CSPG5 antagonist, PRRG2 antagonist, UNC5D antagonist, or PLB1 antagonist reduces the extent and/or severity of HSV-2 infection of the individual relative to infection in the absence of the CSPG5 antagonist, PRRG2 antagonist, UNC5D antagonist, or PLB1 antagonist, respectively.

In some aspects, the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the CSPG5 antagonist, PRRG2 antagonist, UNC5D antagonist, or PLB1 antagonist is a peptide.

In some aspects, the CSPG5 antagonist, PRRG2 antagonist, UNC5D antagonist, or PLB1 antagonist is an antibody or antigen-binding fragment thereof.

In some aspects, (a) the antibody or antigen-binding fragment thereof binds the HSV-2 gG protein and inhibits its binding to CSPG5, PRRG2, and/or UNC5D; or (b) the antibody or antigen-binding fragment thereof binds the HSV-2 gD protein and inhibits its binding to PLB1.

In some aspects, the antibody or antigen-binding fragment thereof binds CSPG5, PRRG2, UNC5D, or PLB1.

In some aspects, (a) the antibody or antigen-binding fragment thereof inhibits the binding of CSPG5, PRRG2, or UNC5D to the HSV-2 gG protein; or (b) the antibody or antigen-binding fragment thereof inhibits the binding of PLB1 to the HSV-2 gD protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the individual has genital herpes or herpes simplex encephalitis.

In some aspects, the individual is a human.

In another aspect, the invention provides a CSPG5 antagonist, a PRRG2 antagonist, a UNC5D antagonist, or a PLB1 antagonist for use as a medicament.

In some aspects, the medicament is for treating an HSV2 infection.

In some aspects, the medicament is for treating genital herpes or herpes simplex encephalitis.

In some aspects, (a) the CSPG5 antagonist results in a decrease in the binding of CSPG5 and the HSV-2 glycoprotein G (gG) protein relative to binding of the two proteins in the absence of the antagonist; (b) the PRRG2 antagonist results in a decrease in the binding of PRRG2 and the HSV-2 gG protein relative to binding of the two proteins in the absence of the antagonist; (c) the UNC5D antagonist results in a decrease in the binding of UNC5D and the HSV-2 gG protein relative to binding of the two proteins in the absence of the antagonist; or (d)the PLB1 antagonist results in a decrease in the binding of PLB1 and the HSV-2 gD protein relative to binding of the two proteins in the absence of the antagonist.

In some aspects, the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the CSPG5 antagonist, PRRG2 antagonist, UNC5D antagonist, or PLB1 antagonist is a peptide.

In some aspects, the CSPG5 antagonist, PRRG2 antagonist, UNC5D antagonist, or PLB1 antagonist is an antibody or antigen-binding fragment thereof.

In some aspects, (a) the antibody or antigen-binding fragment thereof binds the HSV-2 gG protein and inhibits its binding to CSPG5, PRRG2, and/or UNC5D; or (b) the antibody or antigen-binding fragment thereof binds the HSV-2 gD protein and inhibits its binding to PLB1.

In some aspects, the antibody or antigen-binding fragment thereof binds CSPG5, PRRG2, UNC5D, or PLB1.

In some aspects, (a) the antibody or antigen-binding fragment thereof inhibits the binding of CSPG5, PRRG2, or UNC5D to the HSV-2 gG protein; or (b) the antibody or antigen-binding fragment thereof inhibits the binding of PVRL1 or PLB1 to the HSV-2 gD protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides use of a CSPG5 antagonist, a PRRG2 antagonist, a UNC5D antagonist, or a PLB1 antagonist in the manufacture of a medicament for treatment of an HSV-2 infection.

In another aspect, the invention provides use of a CSPG5 antagonist, a PRRG2 antagonist, a UNC5D antagonist, or a PLB1 antagonist in the manufacture of a medicament for treatment of genital herpes or herpes simplex encephalitis.

In another aspect, the invention provides use of a CSPG5 antagonist, a PRRG2 antagonist, a UNC5D antagonist, or a PLB1 antagonist in the manufacture of a medicament for reducing or preventing infection of a cell by HSV-2.

In some aspects, (a) the CSPG5 antagonist results in a decrease in the binding of CSPG5 and the HSV-2 glycoprotein G (gG) protein relative to binding of the two proteins in the absence of the antagonist; (b) the PRRG2 antagonist results in a decrease in the binding of PRRG2 and the HSV-2 gG protein relative to binding of the two proteins in the absence of the antagonist; (c) the UNC5D antagonist results in a decrease in the binding of UNC5D and the HSV-2 gG protein relative to binding of the two proteins in the absence of the antagonist; or (d)the PLB1 antagonist results in a decrease in the binding of PLB1 and the HSV-2 gD protein relative to binding of the two proteins in the absence of the antagonist.

In some aspects, the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the CSPG5 antagonist, PRRG2 antagonist, UNC5D antagonist, or PLB1 antagonist is a peptide.

In some aspects, the CSPG5 antagonist, PRRG2 antagonist, UNC5D antagonist, or PLB1 antagonist is an antibody or antigen-binding fragment thereof.

In some aspects, (a) the antibody or antigen-binding fragment thereof binds the HSV-2 gG protein and inhibits its binding to CSPG5, PRRG2, and/or UNC5D; or (b) the antibody or antigen-binding fragment thereof binds the HSV-2 gD protein and inhibits its binding to PLB1.

In some aspects, the antibody or antigen-binding fragment thereof binds CSPG5, PRRG2, UNC5D, or PLB1.

In some aspects, (a) the antibody or antigen-binding fragment thereof inhibits the binding of CSPG5, PRRG2, or UNC5D to the HSV-2 gG protein; or (b) the antibody or antigen-binding fragment thereof inhibits the binding of PVRL1 or PLB1 to the HSV-2 gD protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides a method of treating an individual having a macacine alphaherpesvirus (MCHV) infection comprising administering to the individual an effective amount of a PILRA antagonist.

In another aspect, the invention provides a method of decreasing MCHV infection in an individual comprising administering to the individual an effective amount of a PILRA antagonist.

In some aspects, the PILRA antagonist results in a decrease in the binding of PILRA and the MCHV glycoprotein G (gG) protein relative to binding of the two proteins in the absence of the antagonist.

In some aspects, the PILRA antagonist reduces the extent and/or severity of MCHV infection of the individual relative to infection in the absence of the PILRA antagonist.

In some aspects, the PILRA antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the PILRA antagonist is a peptide.

In some aspects, the PILRA antagonist is an antibody or antigen-binding fragment thereof.

In some aspects, the antibody or antigen-binding fragment thereof binds the MCHV gG protein and inhibits its binding to PILRA.

In some aspects, the antibody or antigen-binding fragment thereof binds PILRA. In some aspects, the antibody or antigen-binding fragment thereof inhibits the binding of PILRA to the MCHV gG protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the individual has a zoonotic MCHV infection. In some aspects, the individual is a human.

In another aspect, the invention provides a PILRA antagonist for use as a medicament, wherein the medicament is for treating an MCHV infection. In some aspects, the medicament is for treating a zoonotic MCHV infection.

In some aspects, the PILRA antagonist results in a decrease in the binding of PILRA and the MCHV glycoprotein G (gG) protein relative to binding of the two proteins in the absence of the antagonist.

In some aspects, the PILRA antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the PILRA antagonist is a peptide.

In some aspects, the PILRA antagonist is an antibody or antigen-binding fragment thereof.

In some aspects, the antibody or antigen-binding fragment thereof binds the MCHV gG protein and inhibits its binding to PILRA.

In some aspects, the antibody or antigen-binding fragment thereof binds PILRA. In some aspects, the antibody or antigen-binding fragment thereof inhibits the binding of PILRA to the MCHV gG protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides use of a PILRA antagonist in the manufacture of a medicament for treatment of a MCHV infection.

In another aspect, the invention provides use of a PILRA antagonist in the manufacture of a medicament for treatment of a zoonotic MCHV infection.

In another aspect, the invention provides use of a PILRA antagonist in the manufacture of a medicament for reducing or preventing infection of a cell by MCHV.

In some aspects, the PILRA antagonist results in a decrease in the binding of PILRA and the MCHV glycoprotein G (gG) protein relative to binding of the two proteins in the absence of the antagonist.

In some aspects, the PILRA antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the PILRA antagonist is a peptide.

In some aspects, the PILRA antagonist is an antibody or antigen-binding fragment thereof.

In some aspects, the antibody or antigen-binding fragment thereof binds the MCHV gG protein and inhibits its binding to PILRA.

In some aspects, the antibody or antigen-binding fragment thereof binds PILRA. In some aspects, the antibody or antigen-binding fragment thereof inhibits the binding of PILRA to the MCHV gG protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides a method of treating an individual having a human cytomegalovirus (HCMV) infection comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist.

In another aspect, the invention provides a method of decreasing HCMV infection in an individual comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist.

In some aspects, (a) the VEGFR2 antagonist results in a decrease in the binding of VEGFR2 and the HCMV UL6 protein relative to binding of the two proteins in the absence of the antagonist; (b) the MERTK antagonist results in a decrease in the binding of MERTK and the HCMV UL6 protein relative to binding of the two proteins in the absence of the antagonist; (c) the PDGFRa antagonist results in a decrease in the binding of PDGFRa and the HCMV UL6 protein relative to binding of the two proteins in the absence of the antagonist; (d) the KIRREL2 antagonist results in a decrease in the binding of KIRREL2 and the HCMV UL6 protein relative to binding of the two proteins in the absence of the antagonist; (e) the LILRB5 antagonist results in a decrease in the binding of LILRB5 and the HCMV UL9 protein relative to binding of the two proteins in the absence of the antagonist; (f) the ULBP1 antagonist results in a decrease in the binding of ULBP1 and the HCMV UL9 protein relative to binding of the two proteins in the absence of the antagonist; (g) the KIR2DL3 antagonist results in a decrease in the binding of KIR2DL3 and the HCMV UL9 protein relative to binding of the two proteins in the absence of the antagonist; (h) the KIR2DS1 antagonist results in a decrease in the binding of KIR2DS1 and the HCMV UL9 protein relative to binding of the two proteins in the absence of the antagonist; (i) the KIR2DS2 antagonist results in a decrease in the binding of KIR2DS2 and the HCMV UL9 protein relative to binding of the two proteins in the absence of the antagonist; (j) the KIR2DS4 antagonist results in a decrease in the binding of KIR2DS4 and the HCMV UL9 protein relative to binding of the two proteins in the absence of the antagonist; (k) the KIR2DS5 antagonist results in a decrease in the binding of KIR2DS5 and the HCMV UL9 protein relative to binding of the two proteins in the absence of the antagonist; (l) the KIR2DL1 antagonist results in a decrease in the binding of KIR2DL1 and the HCMV UL9 protein relative to binding of the two proteins in the absence of the antagonist; (m) the KIR3DL1 antagonist results in a decrease in the binding of KIR3DL1 and the HCMV UL9 protein relative to binding of the two proteins in the absence of the antagonist; (n) the PRRG2 antagonist results in a decrease in the binding of PRRG2 and the HCMV UL142 protein relative to binding of the two proteins in the absence of the antagonist; (o) the KLRAP1 antagonist results in a decrease in the binding of KLRAP1 and the HCMV UL144 protein relative to binding of the two proteins in the absence of the antagonist; or (p) the SGCA antagonist results in a decrease in the binding of SGCA and the HCMV RL10 protein relative to binding of the two proteins in the absence of the antagonist.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist reduces the extent and/or severity of HCMV infection of the individual relative to infection in the absence of the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist, respectively.

In some aspects, VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof.

In some aspects, (a) the antibody or antigen-binding fragment thereof binds the HCMV UL6 protein and inhibits its binding to VEGFR2, MERTK, PDGFRa, and/or KIRREL2; (b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1; (c) the antibody or antigen-binding fragment thereof binds the HCMV UL142 protein and inhibits its binding to PRRG2; (d) the antibody or antigen-binding fragment thereof binds the HCMV UL144 protein and inhibits its binding to KLRAP1; or (e) the antibody or antigen-binding fragment thereof binds the HCMV RL10 protein and inhibits its binding to SGCA.

In some aspects, the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA.

In some aspects, (a) the antibody or antigen-binding fragment thereof inhibits the binding of VEGFR2, MERTK, PDGFRa, or KIRREL2 to the HCMV UL6 protein; (b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein; (c) the antibody or antigen-binding fragment thereof inhibits the binding of PRRG2 to the HCMV UL142 protein; (d) the antibody or antigen-binding fragment thereof inhibits the binding of KLRAP1 to the HCMV UL144 protein; or (e) the antibody or antigen-binding fragment thereof inhibits the binding of SGCA to the HCMV RL10 protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the HCMV infection is congenital.

In some aspects, the individual has CMV-related allograft rejection.

In some aspects, the individual is a human.

In another aspect, the invention provides a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist for use as a medicament.

In another aspect, the invention provides a VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use as a medicament, wherein the medicament is for treating a HCMV infection.

In another aspect, the invention provides a VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use as a medicament, wherein the medicament is for treating CMV-related allograft rejection.

In some aspects, (a) the VEGFR2 antagonist results in a decrease in the binding of VEGFR2 and the HCMV UL6 protein relative to binding of the two proteins in the absence of the antagonist; (b) the MERTK antagonist results in a decrease in the binding of MERTK and the HCMV UL6 protein relative to binding of the two proteins in the absence of the antagonist; (c) the PDGFRa antagonist results in a decrease in the binding of PDGFRa and the HCMV UL6 protein relative to binding of the two proteins in the absence of the antagonist; (d) the KIRREL2 antagonist results in a decrease in the binding of KIRREL2 and the HCMV UL6 protein relative to binding of the two proteins in the absence of the antagonist; (e) the LILRB5 antagonist results in a decrease in the binding of LILRB5 and the HCMV UL9 protein relative to binding of the two proteins in the absence of the antagonist; (f) the ULBP1 antagonist results in a decrease in the binding of ULBP1 and the HCMV UL9 protein relative to binding of the two proteins in the absence of the antagonist; (g) the KIR2DL3 antagonist results in a decrease in the binding of KIR2DL3 and the HCMV UL9 protein relative to binding of the two proteins in the absence of the antagonist; (h) the KIR2DS1 antagonist results in a decrease in the binding of KIR2DS1 and the HCMV UL9 protein relative to binding of the two proteins in the absence of the antagonist; (i) the KIR2DS2 antagonist results in a decrease in the binding of KIR2DS2 and the HCMV UL9 protein relative to binding of the two proteins in the absence of the antagonist; (j) the KIR2DS4 antagonist results in a decrease in the binding of KIR2DS4 and the HCMV UL9 protein relative to binding of the two proteins in the absence of the antagonist; (k) the KIR2DS5 antagonist results in a decrease in the binding of KIR2DS5 and the HCMV UL9 protein relative to binding of the two proteins in the absence of the antagonist; (l) the KIR2DL1 antagonist results in a decrease in the binding of KIR2DL1 and the HCMV UL9 protein relative to binding of the two proteins in the absence of the antagonist; (m) the KIR3DL1 antagonist results in a decrease in the binding of KIR3DL1 and the HCMV UL9 protein relative to binding of the two proteins in the absence of the antagonist; (n) the PRRG2 antagonist results in a decrease in the binding of PRRG2 and the HCMV UL142 protein relative to binding of the two proteins in the absence of the antagonist; (o) the KLRAP1 antagonist results in a decrease in the binding of KLRAP1 and the HCMV UL144 protein relative to binding of the two proteins in the absence of the antagonist; or (p) the SGCA antagonist results in a decrease in the binding of SGCA and the HCMV RL10 protein relative to binding of the two proteins in the absence of the antagonist.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist reduces the extent and/or severity of HCMV infection of the individual relative to infection in the absence of the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist, respectively.

In some aspects, the inhibitory nucleic acid is an ASO or a siRNA.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof.

In some aspects, (a) the antibody or antigen-binding fragment thereof binds the HCMV UL6 protein and inhibits its binding to VEGFR2, MERTK, PDGFRa, and/or KIRREL2; (b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1; (c) the antibody or antigen-binding fragment thereof binds the HCMV UL142 protein and inhibits its binding to PRRG2; (d) the antibody or antigen-binding fragment thereof binds the HCMV UL144 protein and inhibits its binding to KLRAP1 and/or BTLA; or (e) the antibody or antigen-binding fragment thereof binds the HCMV RL10 protein and inhibits its binding to SGCA.

In some aspects, the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA.

In some aspects, (a) the antibody or antigen-binding fragment thereof inhibits the binding of VEGFR2, MERTK, PDGFRa, or KIRREL2 to the HCMV UL6 protein; (b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein; (c) the antibody or antigen-binding fragment thereof inhibits the binding of PRRG2 to the HCMV UL142 protein; (d) the antibody or antigen-binding fragment thereof inhibits the binding of KLRAP1 or BTLA to the HCMV UL144 protein; or (e) the antibody or antigen-binding fragment thereof inhibits the binding of SGCA to the HCMV RL10 protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for treatment of an HCMV infection.

In another aspect, the invention provides use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for treatment of CMV-related allograft rejection.

In another aspect, the invention provides use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for reducing or preventing infection of a cell by HCMV.

In some aspects, (a) the VEGFR2 antagonist results in a decrease in the binding of VEGFR2 and the HCMV UL6 protein relative to binding of the two proteins in the absence of the antagonist; (b) the MERTK antagonist results in a decrease in the binding of MERTK and the HCMV UL6 protein relative to binding of the two proteins in the absence of the antagonist; (c) the PDGFRa antagonist results in a decrease in the binding of PDGFRa and the HCMV UL6 protein relative to binding of the two proteins in the absence of the antagonist; (d) the KIRREL2 antagonist results in a decrease in the binding of KIRREL2 and the HCMV UL6 protein relative to binding of the two proteins in the absence of the antagonist; (e) the LILRB5 antagonist results in a decrease in the binding of LILRB5 and the HCMV UL9 protein relative to binding of the two proteins in the absence of the antagonist; (f) the ULBP1 antagonist results in a decrease in the binding of ULBP1 and the HCMV UL9 protein relative to binding of the two proteins in the absence of the antagonist; (g) the KIR2DL3 antagonist results in a decrease in the binding of KIR2DL3 and the HCMV UL9 protein relative to binding of the two proteins in the absence of the antagonist; (h) the KIR2DS1 antagonist results in a decrease in the binding of KIR2DS1 and the HCMV UL9 protein relative to binding of the two proteins in the absence of the antagonist; (i) the KIR2DS2 antagonist results in a decrease in the binding of KIR2DS2 and the HCMV UL9 protein relative to binding of the two proteins in the absence of the antagonist; (j) the KIR2DS4 antagonist results in a decrease in the binding of KIR2DS4 and the HCMV UL9 protein relative to binding of the two proteins in the absence of the antagonist; (k) the KIR2DS5 antagonist results in a decrease in the binding of KIR2DS5 and the HCMV UL9 protein relative to binding of the two proteins in the absence of the antagonist; (l) the KIR2DL1 antagonist results in a decrease in the binding of KIR2DL1 and the HCMV UL9 protein relative to binding of the two proteins in the absence of the antagonist; (m) the KIR3DL1 antagonist results in a decrease in the binding of KIR3DL1 and the HCMV UL9 protein relative to binding of the two proteins in the absence of the antagonist; (n) the PRRG2 antagonist results in a decrease in the binding of PRRG2 and the HCMV UL142 protein relative to binding of the two proteins in the absence of the antagonist; (o) the KLRAP1 antagonist results in a decrease in the binding of KLRAP1 and the HCMV UL144 protein relative to binding of the two proteins in the absence of the antagonist; or (p) the SGCA antagonist results in a decrease in the binding of SGCA and the HCMV RL10 protein relative to binding of the two proteins in the absence of the antagonist.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof.

In some aspects, (a) the antibody or antigen-binding fragment thereof binds the HCMV UL6 protein and inhibits its binding to VEGFR2, MERTK, PDGFRa, and/or KIRREL2; (b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1; (c) the antibody or antigen-binding fragment thereof binds the HCMV UL142 protein and inhibits its binding to PRRG2; (d) the antibody or antigen-binding fragment thereof binds the HCMV UL144 protein and inhibits its binding to KLRAP1 and/or BTLA; or (e) the antibody or antigen-binding fragment thereof binds the HCMV RL10 protein and inhibits its binding to SGCA.

In some aspects, the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA.

In some aspects, (a) the antibody or antigen-binding fragment thereof inhibits the binding of VEGFR2, MERTK, PDGFRa, or KIRREL2 to the HCMV UL6 protein; (b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein; (c) the antibody or antigen-binding fragment thereof inhibits the binding of PRRG2 to the HCMV UL142 protein; (d) the antibody or antigen-binding fragment thereof inhibits the binding of KLRAP1 or BTLA to the HCMV UL144 protein; or (e) the antibody or antigen-binding fragment thereof inhibits the binding of SGCA to the HCMV RL10 protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides a method of treating an individual having a Varicella zoster virus (VZV) infection comprising administering to the individual an effective amount of an ICAM1 antagonist, a MUSK antagonist, a HAVCR1 antagonist, a MOG antagonist, or a KIAA0319L antagonist.

In another aspect, the invention provides a method of decreasing VZV infection in an individual comprising administering to the individual an effective amount of an ICAM1 antagonist, a MUSK antagonist, a HAVCR1 antagonist, a MOG antagonist, or a KIAA0319L antagonist.

In some aspects, (a) the ICAM1 antagonist results in a decrease in the binding of ICAM1 and the VZV glycoprotein C (gC) protein relative to binding of the two proteins in the absence of the antagonist; (b) the MUSK antagonist results in a decrease in the binding of MUSK and the VZV glycoprotein B (gB) protein relative to binding of the two proteins in the absence of the antagonist; (c) the HAVCR1 antagonist results in a decrease in the binding of HAVCR1 and the VZV gB protein relative to binding of the two proteins in the absence of the antagonist; (d) the MOG antagonist results in a decrease in the binding of MOG and the VZV glycoprotein I (gI) protein relative to binding of the two proteins in the absence of the antagonist; or (e) the KIAA0319L antagonist results in a decrease in the binding of KIAA0319L and the VZV gI protein relative to binding of the two proteins in the absence of the antagonist.

In some aspects, the ICAM1 antagonist, MUSK antagonist, HAVCR1 antagonist, MOG antagonist, or KIAA0319L antagonist reduces the extent and/or severity of VZV infection of the individual relative to infection in the absence of the ICAM1 antagonist, MUSK antagonist, HAVCR1 antagonist, MOG antagonist, or KIAA0319L antagonist, respectively.

In some aspects, the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the ICAM1 antagonist, MUSK antagonist, HAVCR1 antagonist, MOG antagonist, or KIAA0319L antagonist is a peptide.

In some aspects, the ICAM1 antagonist, MUSK antagonist, HAVCR1 antagonist, MOG antagonist, or KIAA0319L antagonist is an antibody or antigen-binding fragment thereof.

In some aspects, (a) the antibody or antigen-binding fragment thereof binds the VZV gC protein and inhibits its binding to ICAM1; (b) the antibody or antigen-binding fragment thereof binds the VZV gB protein and inhibits its binding to MUSK and/or HAVCR1; or (c) the antibody or antigen-binding fragment thereof binds the VZV gI protein and inhibits its binding to MOG and/or KIAA0319L.

In some aspects, the antibody or antigen-binding fragment thereof binds ICAM1, MUSK, HAVCR1, MOG, or KIAA0319L.

In some aspects, (a) the antibody or antigen-binding fragment thereof inhibits the binding of ICAM1 to the VZV gC protein; (b) the antibody or antigen-binding fragment thereof inhibits the binding of MUSK or HAVCR1 to the VZV gB protein; or (c) the antibody or antigen-binding fragment thereof inhibits the binding of MOG or KIAA0319L to the VZV gI protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the individual has chicken pox or shingles.

In some aspects, the individual is a human.

In another aspect, the invention provides a MOG antagonist, a MUSK antagonist, or a KIAA0319L antagonist for use as a medicament.

In another aspect, the invention provides a ICAM1 antagonist, MUSK antagonist, HAVCR1 antagonist, MOG antagonist, or KIAA0319L antagonist for use as a medicament, wherein the medicament is for treating a VZV infection.

In another aspect, the invention provides an ICAM1 antagonist, MUSK antagonist, HAVCR1 antagonist, MOG antagonist, or KIAA0319L antagonist for use as a medicament, wherein the medicament is for treating chicken pox or shingles.

In some aspects, (a) the ICAM1 antagonist results in a decrease in the binding of ICAM1 and the VZV glycoprotein C (gC) protein relative to binding of the two proteins in the absence of the antagonist; (b) the MUSK antagonist results in a decrease in the binding of MUSK and the VZV glycoprotein B (gB) protein relative to binding of the two proteins in the absence of the antagonist; (c) the HAVCR1 antagonist results in a decrease in the binding of HAVCR1 and the VZV gB protein relative to binding of the two proteins in the absence of the antagonist; (d) the MOG antagonist results in a decrease in the binding of

MOG and the VZV glycoprotein I (gI) protein relative to binding of the two proteins in the absence of the antagonist; or (e) the KIAA0319L antagonist results in a decrease in the binding of KIAA0319L and the VZV gI protein relative to binding of the two proteins in the absence of the antagonist.

In some aspects, the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the ICAM1 antagonist, MUSK antagonist, HAVCR1 antagonist, MOG antagonist, or KIAA0319L antagonist is a peptide.

In some aspects, the ICAM1 antagonist, MUSK antagonist, HAVCR1 antagonist, MOG antagonist, or KIAA0319L antagonist is an antibody or antigen-binding fragment thereof.

In some aspects, (a) the antibody or antigen-binding fragment thereof binds the VZV gC protein and inhibits its binding to ICAM1; (b) the antibody or antigen-binding fragment thereof binds the VZV gB protein and inhibits its binding to MUSK and/or HAVCR1; or (c) the antibody or antigen-binding fragment thereof binds the VZV gI protein and inhibits its binding to MOG and/or KIAA0319L.

In some aspects, the antibody or antigen-binding fragment thereof binds ICAM1, MUSK, HAVCR1, MOG, or KIAA0319L.

In some aspects, (a) the antibody or antigen-binding fragment thereof inhibits the binding of ICAM1 to the VZV gC protein; (b) the antibody or antigen-binding fragment thereof inhibits the binding of MUSK or HAVCR1 to the VZV gB protein; or (c) the antibody or antigen-binding fragment thereof inhibits the binding of MOG or KIAA0319L to the VZV gI protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides use of an ICAM1 antagonist, a MUSK antagonist, a HAVCR1 antagonist, a MOG antagonist, or a KIAA0319L antagonist in the manufacture of a medicament for treatment of a VZV infection.

In another aspect, the invention provides use of an ICAM1 antagonist, a MUSK antagonist, a HAVCR1 antagonist, a MOG antagonist, or a KIAA0319L antagonist in the manufacture of a medicament for treatment of chicken pox or shingles.

In another aspect, the invention provides use of an ICAM1 antagonist, a MUSK antagonist, a HAVCR1 antagonist, a MOG antagonist, or a KIAA0319L antagonist in the manufacture of a medicament for reducing or preventing infection of a cell by VZV.

In some aspects, (a) the ICAM1 antagonist results in a decrease in the binding of ICAM1 and the VZV glycoprotein C (gC) protein relative to binding of the two proteins in the absence of the antagonist; (b) the MUSK antagonist results in a decrease in the binding of MUSK and the VZV glycoprotein B (gB) protein relative to binding of the two proteins in the absence of the antagonist; (c) the HAVCR1 antagonist results in a decrease in the binding of HAVCR1 and the VZV gB protein relative to binding of the two proteins in the absence of the antagonist; (d) the MOG antagonist results in a decrease in the binding of MOG and the VZV glycoprotein I (gI) protein relative to binding of the two proteins in the absence of the antagonist; or (e) the KIAA0319L antagonist results in a decrease in the binding of KIAA0319L and the VZV gI protein relative to binding of the two proteins in the absence of the antagonist.

In some aspects, the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the ICAM1 antagonist, MUSK antagonist, HAVCR1 antagonist, MOG antagonist, or KIAA0319L antagonist is a peptide.

In some aspects, the ICAM1 antagonist, MUSK antagonist, HAVCR1 antagonist, MOG antagonist, or KIAA0319L antagonist is an antibody or antigen-binding fragment thereof.

In some aspects, (a) the antibody or antigen-binding fragment thereof binds the VZV gC protein and inhibits its binding to ICAM1; (b) the antibody or antigen-binding fragment thereof binds the VZV gB protein and inhibits its binding to MUSK and/or HAVCR1; or (c) the antibody or antigen-binding fragment thereof binds the VZV gI protein and inhibits its binding to MOG and/or KIAA0319L.

In some aspects, the antibody or antigen-binding fragment thereof binds ICAM1, MUSK, HAVCR1, MOG, or KIAA0319L.

In some aspects, (a) the antibody or antigen-binding fragment thereof inhibits the binding of ICAM1 to the VZV gC protein; (b) the antibody or antigen-binding fragment thereof inhibits the binding of MUSK or HAVCR1 to the VZV gB protein; or (c) the antibody or antigen-binding fragment thereof inhibits the binding of MOG or KIAA0319L to the VZV gI protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides a method of treating an individual having a human herpesvirus 8 (HHV8) infection comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

In another aspect, the invention provides a method of decreasing HHV8 infection in an individual comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

In some aspects, (a) the KLRAP1 antagonist results in a decrease in the binding of KLRAP1 and the HHV8 K14 protein relative to binding of the two proteins in the absence of the antagonist; (b) the LILRB1 antagonist results in a decrease in the binding of LILRB1 and the HHV8 KCP protein relative to binding of the two proteins in the absence of the antagonist; (c) the CLEC4G antagonist results in a decrease in the binding of CLEC4G and the HHV8 KCP protein relative to binding of the two proteins in the absence of the antagonist; (d) the FLRT1 antagonist results in a decrease in the binding of FLRT1 and the HHV8 KCP protein relative to binding of the two proteins in the absence of the antagonist; (e) the FLRT2 antagonist results in a decrease in the binding of FLRT2 and the HHV8 KCP protein relative to binding of the two proteins in the absence of the antagonist; or (f) the FLRT3 antagonist results in a decrease in the binding of FLRT3 and the HHV8 KCP protein relative to binding of the two proteins in the absence of the antagonist.

In some aspects, the KLRAP1 antagonist, LILRB1 antagonist, CLEC4G antagonist, FLRT1 antagonist, FLRT2 antagonist, or FLRT3 antagonist reduces the extent and/or severity of HHV8 infection of the individual relative to infection in the absence of the KLRAP1 antagonist, LILRB1 antagonist, CLEC4G antagonist, FLRT1 antagonist, FLRT2 antagonist, or FLRT3 antagonist, respectively.

In some aspects, the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the KLRAP1 antagonist, LILRB1 antagonist, CLEC4G antagonist, FLRT1 antagonist, FLRT2 antagonist, or FLRT3 antagonist is a peptide.

In some aspects, the KLRAP1 antagonist, LILRB1 antagonist, CLEC4G antagonist, FLRT1 antagonist, FLRT2 antagonist, or FLRT3 antagonist is an antibody or antigen-binding fragment thereof.

In some aspects, (a) the antibody or antigen-binding fragment thereof binds the HHV8 K14 protein and inhibits its binding to KLRAP1; or (b) the antibody or antigen-binding fragment thereof binds the HHV8 KCP protein and inhibits its binding to LILRB1, CLEC4G, FLRT1, FLRT2, and/or FLRT3.

In some aspects, the antibody or antigen-binding fragment thereof binds KLRAP1, LILRB1, CLEC4G, FLRT1, FLRT2, or FLRT3.

In some aspects, (a) the antibody or antigen-binding fragment thereof inhibits the binding of KLRAP1 to the HHV8 K14 protein; or (b) the antibody or antigen-binding fragment thereof inhibits the binding of LILRB1, CLEC4G, FLRT1, FLRT2, or FLRT3 to the HHV8 KCP protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the individual has Kaposi's sarcoma, primary effusion lymphoma, HHV8-associated multicentric Castleman's disease, or KSHV inflammatory cytokine syndrome.

In some aspects, the individual is a human.

In another aspect, the invention provides a KLRAP1 antagonist, a CLEC4G antagonist, a FLRT1 antagonist, a FLRT2 antagonist, or a FLRT3 antagonist for use as a medicament.

In another aspect, the invention provides a KLRAP1 antagonist, LILRB1 antagonist, CLEC4G antagonist, FLRT1 antagonist, FLRT2 antagonist, or FLRT3 antagonist for use as a medicament, wherein the medicament is for treating an HHV8 infection.

In another aspect, the invention provides a KLRAP1 antagonist, LILRB1 antagonist, CLEC4G antagonist, FLRT1 antagonist, FLRT2 antagonist, or FLRT3 antagonist for use as a medicament, wherein the medicament is for treating Kaposi's sarcoma, primary effusion lymphoma, HHV8-associated multicentric Castleman's disease, or KSHV inflammatory cytokine syndrome.

In some aspects, (a) the KLRAP1 antagonist results in a decrease in the binding of KLRAP1 and the HHV8 K14 protein relative to binding of the two proteins in the absence of the antagonist; (b) the LILRB1 antagonist results in a decrease in the binding of LILRB1 and the HHV8 KCP protein relative to binding of the two proteins in the absence of the antagonist; (c) the CLEC4G antagonist results in a decrease in the binding of CLEC4G and the HHV8 KCP protein relative to binding of the two proteins in the absence of the antagonist; (d) the FLRT1 antagonist results in a decrease in the binding of FLRT1 and the HHV8 KCP protein relative to binding of the two proteins in the absence of the antagonist; (e) the FLRT2 antagonist results in a decrease in the binding of FLRT2 and the HHV8 KCP protein relative to binding of the two proteins in the absence of the antagonist; or (f) the FLRT3 antagonist results in a decrease in the binding of FLRT3 and the HHV8 KCP protein relative to binding of the two proteins in the absence of the antagonist.

In some aspects, the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the KLRAP1 antagonist, LILRB1 antagonist, CLEC4G antagonist, FLRT1 antagonist, FLRT2 antagonist, or FLRT3 antagonist is a peptide.

In some aspects, the KLRAP1 antagonist, LILRB1 antagonist, CLEC4G antagonist, FLRT1 antagonist, FLRT2 antagonist, or FLRT3 antagonist is an antibody or antigen-binding fragment thereof.

In some aspects, (a) the antibody or antigen-binding fragment thereof binds the HHV8 K14 protein and inhibits its binding to KLRAP1; or (b) the antibody or antigen-binding fragment thereof binds the HHV8 KCP protein and inhibits its binding to LILRB1, CLEC4G, FLRT1, FLRT2, and/or FLRT3.

In some aspects, the antibody or antigen-binding fragment thereof binds KLRAP1, LILRB1, CLEC4G, FLRT1, FLRT2, or FLRT3.

In some aspects, (a) the antibody or antigen-binding fragment thereof inhibits the binding of KLRAP1 to the HHV8 K14 protein; or (b) the antibody or antigen-binding fragment thereof inhibits the binding of LILRB1, CLEC4G, FLRT1, FLRT2, or FLRT3 to the HHV8 KCP protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the invention provides use of a KLRAP1 antagonist, a LILRB1 antagonist, a CLEC4G antagonist, a FLRT1 antagonist, a FLRT2 antagonist, or a FLRT3 antagonist in the manufacture of a medicament for treatment of an HHV8 infection.

In another aspect, the invention provides use of a KLRAP1 antagonist, a LILRB1 antagonist, a CLEC4G antagonist, a FLRT1 antagonist, a FLRT2 antagonist, or a FLRT3 antagonist in the manufacture of a medicament for treatment of Kaposi's sarcoma, primary effusion lymphoma, HHV8-associated multicentric Castleman's disease, or KSHV inflammatory cytokine syndrome.

In another aspect, the invention provides use of a KLRAP1 antagonist, a LILRB1 antagonist, a CLEC4G antagonist, a FLRT1 antagonist, a FLRT2 antagonist, or a FLRT3 antagonist in the manufacture of a medicament for reducing or preventing infection of a cell by HHV8.

In some aspects, (a) the KLRAP1 antagonist results in a decrease in the binding of KLRAP1 and the HHV8 K14 protein relative to binding of the two proteins in the absence of the antagonist; (b) the LILRB1 antagonist results in a decrease in the binding of LILRB1 and the HHV8 KCP protein relative to binding of the two proteins in the absence of the antagonist; (c) the CLEC4G antagonist results in a decrease in the binding of CLEC4G and the HHV8 KCP protein relative to binding of the two proteins in the absence of the antagonist; (d) the FLRT1 antagonist results in a decrease in the binding of FLRT1 and the HHV8 KCP protein relative to binding of the two proteins in the absence of the antagonist; (e) the FLRT2 antagonist results in a decrease in the binding of FLRT2 and the HHV8 KCP protein relative to binding of the two proteins in the absence of the antagonist; or (f) the FLRT3 antagonist results in a decrease in the binding of FLRT3 and the HHV8 KCP protein relative to binding of the two proteins in the absence of the antagonist.

In some aspects, the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

In some aspects, the inhibitory nucleic acid is an ASO or a siRNA.

In some aspects, the KLRAP1 antagonist, LILRB1 antagonist, CLEC4G antagonist, FLRT1 antagonist, FLRT2 antagonist, or FLRT3 antagonist is a peptide.

In some aspects, the KLRAP1 antagonist, LILRB1 antagonist, CLEC4G antagonist, FLRT1 antagonist, FLRT2 antagonist, or FLRT3 antagonist is an antibody or antigen-binding fragment thereof.

In some aspects, (a) the antibody or antigen-binding fragment thereof binds the HHV8 K14 protein and inhibits its binding to KLRAP1; or (b) the antibody or antigen-binding fragment thereof binds the HHV8 KCP protein and inhibits its binding to LILRB1, CLEC4G, FLRT1, FLRT2, and/or FLRT3.

In some aspects, the antibody or antigen-binding fragment thereof binds KLRAP1, LILRB1, CLEC4G, FLRT1, FLRT2, or FLRT3.

In some aspects, (a) the antibody or antigen-binding fragment thereof inhibits the binding of KLRAP1 to the HHV8 K14 protein; or (b) the antibody or antigen-binding fragment thereof inhibits the binding of LILRB1, CLEC4G, FLRT1, FLRT2, or FLRT3 to the HHV8 KCP protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In another aspect, the disclosure features a method of identifying a modulator of the interaction between the HCMV UL6 protein and MERTK or VEGFR2, the method comprising (a) providing a candidate modulator; (b) contacting the HCMV UL6 protein with MERTK or VEGFR2 in the presence or absence of the candidate modulator under conditions permitting the binding of the HCMV UL6 protein to MERTK or VEGFR2; and (c) measuring the binding of the HCMV UL6 protein to MERTK or VEGFR2, wherein an increase or decrease in binding in the presence of the candidate modulator relative to binding in the absence of the candidate modulator identifies the candidate modulator as a modulator of the interaction between the HCMV UL6 protein and MERTK or VEGFR2.

In another aspect, the disclosure features a method of identifying a modulator of a downstream activity of the HCMV UL6 protein, the method comprising (a) providing a candidate modulator; (b) contacting the HCMV UL6 protein with MERTK or VEGFR2 in the presence or absence of the candidate modulator under conditions permitting the binding of the HCMV UL6 protein to MERTK or VEGFR2; and (c) measuring a downstream activity of the HCMV UL6 protein, wherein a change in the downstream activity in the presence of the candidate modulator relative to the downstream activity in the absence of the candidate modulator identifies the candidate modulator as a modulator of the downstream activity of the HCMV UL6 protein.

In another aspect, the disclosure features a method of identifying a modulator of a downstream activity of MERTK or VEGFR2, the method comprising (a) providing a candidate modulator; (b) contacting MERTK or VEGFR2 with the HCMV UL6 protein in the presence or absence of the candidate modulator under conditions permitting the binding of MERTK or VEGFR2 to the HCMV UL6 protein; and (c) measuring a downstream activity of MERTK or VEGFR2, wherein a change in the downstream activity in the presence of the candidate modulator relative to the downstream activity in the absence of the candidate modulator identifies the candidate modulator as a modulator of the downstream activity of MERTK or VEGFR2.

In some aspects, the increase or decrease in binding is at least 70%, as measured by a surface plasmon resonance (SPR) assay, a BLI assay, or an enzyme-linked immunosorbent assay (ELISA).

In some aspects, the modulator is an inhibitor of the downstream activity of the HCMV UL6 protein or MERTK or VEGFR2.

In some aspects, the modulator is an activator of the downstream activity of the HCMV UL6 protein or MERTK or VEGFR2.

In some aspects, the change in the downstream activity is a decrease in the amount, strength, or duration of the downstream activity.

In some aspects, the change in the downstream activity is an increase in the amount, strength, or duration of the downstream activity.

In some aspects, the downstream activity is infection of a cell by HCMV.

In some aspects, infection is decreased in the presence of the modulator. In some aspects, infection is decreased by at least 40%, as measured in a viral infection assay or a viral entry assay.

In some aspects, the downstream activity is angiogenesis. In some aspects, angiogenesis is decreased by at least 40%, as measured in a tube formation assay.

In some aspects, the modulator is an inhibitor of a downstream activity of the HCMV UL6 protein or MERTK, and the downstream activity is phosphorylation of MERTK. In some aspects, phosphorylation of MERTK is decreased by at least 40%, as measured using a Western blot or ELISA.

In some aspects, the modulator is a small molecule, an antibody or antigen-binding fragment thereof, a peptide, a mimic, an antisense oligonucleotide, or a small interfering RNA (siRNA).

In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the antibody or antigen-binding fragment thereof binds the HCMV UL6 protein. In some aspects, the antibody or antigen-binding fragment thereof binds MERTK or VEGFR2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a plot showing the results of a screen for interaction between the HCMV protein UL6 (as a pentamerized query protein) and the STM receptor library.

FIG. 2 is a plot showing the results of a screen for interaction between the HCMV protein UL9 (as a pentamerized query protein) and the STM receptor library.

FIG. 3 is a plot showing the results of a screen for interaction between the human herpesvirus 8 (HHV8; also called Kaposi's sarcoma herpesvirus (KSHV)) protein KCP (as a pentamerized query protein) and the STM receptor library.

FIG. 4 is a plot showing the results of a screen for interaction between the Herpes simplex virus 2 (HSV-2) glycoprotein G (gG) (as a pentamerized query protein) and the STM receptor library.

FIG. 5 is a plot showing the results of a screen for interaction between the Varicella zoster virus (VZV) glycoprotein B (gB) (as a pentamerized query protein) and the STM receptor library.

FIG. 6 is a plot showing the results of a screen for interaction between the VZV glycoprotein C (gC) (as a pentamerized query protein) and the STM receptor library.

FIG. 7 is a plot showing the results of a screen for interaction between the HSV-2 glycoprotein D (gD) (as a pentamerized query protein) and the STM receptor library.

FIG. 8 is a plot showing the results of a screen for interaction between the macacine alphaherpesvirus (MCHV) glycoprotein G (gG) (as a pentamerized query protein) and the STM receptor library.

FIG. 9 is a plot showing the results of a screen for interaction between the VZV glycoprotein I (gI) (as a pentamerized query protein) and the STM receptor library.

FIG. 10 is a plot showing the results of a screen for interaction between the HCMV protein RL10 (as a pentamerized query protein) and the STM receptor library.

FIG. 11 is a plot showing the results of a screen for interaction between the HCMV protein UL142 (as a pentamerized query protein) and the STM receptor library.

FIG. 12 is a plot showing the results of a screen for interaction between the HCMV protein UL144 (as a pentamerized query protein) and the STM receptor library.

FIG. 13 is a plot showing the results of a screen for interaction between the HHV8 protein K14 (as a pentamerized query protein) and the STM receptor library.

FIG. 14A is a set of micrographs showing cell-based immunofluorescence validation of the interaction between the VZV protein gC (expressed as a gD-GPI construct) and ICAM1 (expressed as a tetramer).

FIG. 14B is a set of micrographs showing cell-based immunofluorescence validation of the interaction between the VZV protein gC (captured on beads or assayed directly in supernatants (sups)) and ICAM1 (expressed as a gD-GPI construct or a full-length protein).

FIG. 15 is a set of micrographs showing cell-based immunofluorescence validation of the interaction between the VZV protein gB (captured on beads or assayed directly in supernatants) and MUSK (expressed as a g D-GPI construct or a full-length protein).

FIG. 16 is a set of micrographs showing cell-based immunofluorescence validation of the interaction between the VZV protein gB (captured on beads or assayed directly in supernatants) and HAVCR1 (expressed as a gD-GPI construct or a full-length protein).

FIG. 17 is a set of micrographs showing cell-based immunofluorescence validation of the interaction between the HHV8 protein KCP (expressed as a gD-GPI construct or a full-length protein) and LILRB1, FLRT1, FLRT2, and FLRT3 (in supernatants).

FIG. 18 is a pair of micrographs showing Cos7 cell-based immunofluorescence validation of the interaction between the HCMV protein UL6 and PDGFRA (expressed as an Fc fusion protein.

FIG. 19 is a pair of micrographs showing Cos7 cell-based immunofluorescence validation of the interaction between the HCMV protein UL6 and MERTK.

FIG. 20 is a pair of micrographs showing Cos7 cell-based immunofluorescence validation of the interaction between the HSV-2 protein gG and UNC5D-Fc.

FIG. 21 is a set of micrographs showing cell-based immunofluorescence validation of the interaction between the HCMV protein UL9 (expressed as a gD-GPI construct or a full-length protein) and the indicated proteins.

FIG. 22 is a set of micrographs showing cell-based immunofluorescence validation of the interaction between the VZV protein gC and ICAM1 (expressed as a g D-GPI construct or a full-length protein).

FIG. 23 is a scatter plot showing the results of an Extracellular Protein Microarray technology screen for proteins that interact with the hCMV protein UL6. The human receptors VEGFR2 (KDR) and MERTK (MER) were detected as high scoring hits. The HCMV UL6 ectodomain was expressed as a recombinant Fc-tagged protein and was screened against two libraries consisting of ˜1,500 human proteins. Scatter plot represents two replicate microarray data sets (array 1, array 2), where dots represent average scores for binding of HCMV UL6 to each human protein in the library. The lower left square represents an empirically set cut off of ˜4 and contains all proteins from the library that were not detected as “hits” for UL6. Dark gray dots represent two different preparations of MERTK present in the library that were detected as high-scoring hits for UL6. Light gray dots represent two different preparations of VEGFR2 present in the library that were detected as high-scoring hits for UL6.

FIG. 24A is a surface plasmon resonance (SPR) plot showing binding of MERTK (expressed as a His-tagged ectodomain) to recombinant UL6 (expressed as a Fc-fused ectodomain: UL6-Fc). UL6-Fc was immobilized on a sensor chip. Binding to MERTK, used as soluble analyte, was assessed by SPR. MERTK binding was tested at 0, 10, 50, 100 and 200 nanomolar (nM) concentration, respectively (bottom to top).

FIG. 24B is a SPR plot showing binding of VEGFR2 (expressed as a monomeric ectodomain) to UL6-Fc. Recombinant VEGFR2 was immobilized on sensor chips. Binding to UL6-Fc, used as soluble analyte, was tested by surface plasmon resonance. UL6 binding was tested at 10, 20, 50, 100 and 200 nM concentration (bottom to top).

FIG. 25 is a SPR plot showing binding of UL6-Fc (provided at 5 nM, 10 nM, or 100 nM), Gas6 (hGas6 His R&D; provided at 100 nM), or a control Fc-tagged protein (Control viral prot-Fc; provided at 100 nM) to MERTK-Fc (hMER-Fc R&D). Recombinant MERTK, expressed as a recombinant ectodomain fused to a Fc tag, was immobilized on GLC sensor chips using standard amino coupling chemistry. UL6-Fc, Gas6, and a control viral protein (ectodomain-Fc) were tested for binding as soluble analytes, injected at the indicated concentrations

FIG. 26 is a set of SPR plots showing binding of UL6-Fc (provided at 5 nM, 10 nM, or 100 nM), Gas6 (hGas6 His R&D; provided at 100 nM), or a control Fc-tagged protein (Control viral prot-Fc; provided at 100 nM) to mouse MERTK-Fc (MER-Fc) (left panel), human MERTK-Fc (center panel), or a control viral protein ectodomain (control-Fc). MERTK or a control Fc protein were immobilized on GLC sensor chips using standard amino coupling chemistry. UL6-Fc, Gas6, and the control viral protein were tested for binding as soluble analytes, injected at the indicated concentrations.

FIG. 27A is a set of flow cytometry histograms showing expression of the receptor MERTK (MER) on the surface of human umbilical vein endothelial cells (HUVECs). Left histogram: background signal (unlabeled cells). Dashed line histogram: isotype control staining. Right histogram: expression of MERTK on the cell surface, as measured using a commercial anti-MERTK antibody ((Cat No. #AF891, R&D).

FIG. 27B is a set of flow cytometry histograms showing binding of recombinant UL6-Fc to the surface of HUVECs. UL6 was incubated with the cells at 4° C. at the concentrations indicated, and binding was measured using a fluorophore-conjugated anti-tag (anti-Fc) antibody. Left solid histogram: background signal (unlabeled cells). Dashed line histogram: binding of a Fc-tagged control protein (provided at 200 nM). Right histograms: binding of UL6-Fc provided at 10 nM, 100 nM, or 200 nM to cells.

FIG. 28 is a photomicrograph of a Western blot showing the levels of phosphoMER (pMer), phosphoAKT (pAKT⁴⁷³), phosphoERK42/44 (pERK42/44), phosphoMEK (pMEK), and actin in HUVECs stimulated with an Fc control protein at 100 nM, VEGFA at 20 ng/mL, UL6-Fc at 10 nM, UL6-Fc at 100 nM, or Gas6 at 400 nM or mock-stimulated cells. IB: immunoblotting.

FIG. 29A is a schematic diagram showing the design of a tube formation assay used to evaluate angiogenesis in vivo. Endothelial cells are grown in 3D matrices (fibrin gel), and their ability to form capillary-like structures (or tubules) is assessed. Beads comprising HUVECs are provided.

FIG. 29B is a set of photomicrographs showing representative images from HUVEC tube formation assays performed as shown in FIG. 29A. Assays were performed in the presence of UL6-Fc, VEGFA (used as a positive control for tube formation), or a control Fc-tagged protein.

FIG. 30 is a schematic diagram showing the design of a biolayer interferometry (BLI) assay for competition of UL6 and Gas6 for binding to MERTK and a graph showing the results of the assay.

FIG. 31 is a set of photomicrographs showing representative images from HUVEC tube formation assays performed as shown in FIG. 29A. Cells were treated with MERTK (MER) siRNA or a control siRNA. Non-treated cells are shown as a control. Assays were performed in the presence of UL6-Fc, VEGFA (used as a positive control for tube formation), or a control Fc-tagged protein.

### DETAILED DESCRIPTION OF THE INVENTION

### I. DEFINITIONS

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) aspects that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising,” will be understood to imply the inclusion of a stated integer or group of integers but not the exclusion of any other integer or group of integers.

The term “patient” herein refers to a human patient.

An “effective amount” refers to an amount of an agent (e.g., a therapeutic agent) that is effective to bring about a therapeutic/prophylactic benefit (e.g., as described herein) that is not outweighed by unwanted/undesirable side effects.

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In one embodiment, the formulation is for intravenous (iv) administration. In another embodiment, the formulation is for subcutaneous (sc) administration.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein any form of the protein that results from processing in the cell. The term also encompasses naturally occurring variants of the protein, e.g., splice variants or allelic variants, e.g., amino acid substitution mutations or amino acid deletion mutations. The term also includes isolated regions or domains of the protein, e.g., the extracellular domain (ECD).

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “single transmembrane receptor,” “single-pass transmembrane receptor,” or “STM receptor,” as used herein, refers to a protein having a single transmembrane domain. In some aspects, the STM receptor is expressed on the cell surface. Exemplary STM receptors are provided in Martinez-Martin et al., Cell, 174(5): 1158-1171, 2018 and Clark et al., Genome Res, 13: 2265-2270, 2003. In some aspects, the STM protein has the UniProt annotation “leucine-rich,” “cysteine-rich,” “ITIM/ITAM” (immunoreceptor tyrosine-based inhibition motif/immunoreceptor tyrosine-based activation motif), “TNFR” (tumor necrosis factor receptor), “TLR/ILR” (Toll-like receptor/interleukin receptor), “semaphorin,” “Kinase-like,” “Ig-like” (immunoglobulin-like), “fibronectin,” “ephrin,” “EGF,” “cytokineR,” or “cadherin.” STM receptors may be identified based on, e.g., the presence of a signal peptide or a predicted transmembrane region in the amino acid sequence. In some aspects, the STM receptor is expressed as an extracellular domain.

As used herein, the term “extracellular domain” or “ECD” refers to a protein domain that is predicted to be localized outside of the outer plasma membrane of the cell. In some instances, the ECD is an ECD of a receptor, e.g., a STM receptor. In some aspects, the ECD is an ECD of a herpesvirus protein. In some aspects, the boundaries of the extracellular domain may be identified by prediction of domains that indicate that the protein crosses the plasma membrane, e.g., a transmembrane domain (e.g., a transmembrane helix). In some aspects, the presence of an extracellular domain may be predicted by the presence of a domain, sequence, or motif that indicates that the protein is trafficked to the plasma membrane, e.g., a signal sequence or a glycosylphosphatidylinositol (GPI) linkage site. In some aspects, the boundaries of the ECD are determined according to UniProt annotations. In some aspects, the ECD is soluble. In some aspects, the extracellular domain is expressed in the context of a full-length protein. In other aspects, the extracellular domain is expressed as an isolated extracellular domain, e.g., a sequence of amino acid residues comprising only the amino acid residues of a protein that are predicted to be extracellular.

As used herein, the term “extracellular domain” or “ECD” refers to a protein domain that is predicted to be localized outside of the outer plasma membrane of the cell. In some instances, the ECD is an ECD of a receptor, e.g., a STM receptor. In some aspects, the ECD is an ECD of a herpesvirus protein. In some aspects, the boundaries of the extracellular domain may be identified by prediction of domains that indicate that the protein crosses the plasma membrane, e.g., a transmembrane domain (e.g., a transmembrane helix). In some aspects, the presence of an extracellular domain may be predicted by the presence of a domain, sequence, or motif that indicates that the protein is trafficked to the plasma membrane, e.g., a signal sequence or a glycosylphosphatidylinositol (GPI) linkage site. In some aspects, the boundaries of the ECD are determined according to UniProt annotations. In some aspects, the ECD is soluble. In some aspects, the extracellular domain is expressed in the context of a full-length protein. In other aspects, the extracellular domain is expressed as an isolated extracellular domain, e.g., a sequence of amino acid residues comprising only the amino acid residues of a protein that are predicted to be extracellular.

In some aspects, the isolated ECD is included in a fusion protein. In some aspects, inclusion in a fusion protein increases solubility, ease of expression, ease of capture (e.g., on a protein A-coated plate), multimerization, or some other desirable property of the ECD. In some aspects, the ECD or ECD fusion protein is a monomer. In other aspects, the ECD or ECD fusion protein is a multimer, e.g., a tetramer or a pentamer. In some aspects, the ECD is fused to a human IgG. In some aspects, the ECD is fused to a human Fc tag. In some aspects, the ECD is fused to an Avidity AVITAG™ (Avi tag). In some aspects, the ECD is fused to a polyhistidine (His) tag. In some aspects, the ECD is fused to a glycoprotein D (gD) tag and a glycosylphosphatidylinositol (GPI) linker, e.g., a gD-GPI tag. In other aspects, the ECD is fused to the pentamerization domain of rat cartilaginous oligomeric matrix protein (COMP) and the β-lactamase protein, e.g., as described in Bushell et al., Genome Res, 18: 622-630, 2008. In some aspects, the ECD fusion protein further includes a cleavage sequence, e.g., a TEV cleavage sequence, to allow removal of one or more domains. In some instances, an ECD fusion protein having an Avi tag and an Fc tag cleavable at a cleavage sequence is further processed to remove the Fc tag, to biotinylate the Avi tag, and to fuse the biotinylated ECD fusion protein to a fluorescent streptavidin (SA), e.g., to form a tetramerized ECD fusion protein. In some instances, the isolated ECD or ECD fusion protein is purified.

As used herein, a “modulator” is an agent that modulates (e.g., increases, decreases, activates, or inhibits) a given biological activity, e.g., an interaction or a downstream activity resulting from an interaction. A modulator or candidate modulator may be, e.g., a small molecule, an antibody, an antigen-binding fragment (e.g., a bis-Fab, an Fv, a Fab, a Fab′-SH, a F(ab′)₂, a diabody, a linear antibody, an scFv, an ScFab, a VH domain, or a VHH domain), a peptide, a mimic, an antisense oligonucleotide, or an inhibitory nucleic acid (e.g., an antisense oligonucleotide (ASO) or a small interfering RNA (siRNA)).

By “increase” or “activate” is meant the ability to cause an overall increase, for example, of 20% or greater, of 50% or greater, or of 75%, 85%, 90%, or 95% or greater. In certain aspects, increase or activate can refer to a downstream activity of a protein-protein interaction.

By “reduce” or “inhibit” is meant the ability to cause an overall decrease, for example, of 20% or greater, of 50% or greater, or of 75%, 85%, 90%, or 95% or greater. In certain aspects, reduce or inhibit can refer to a downstream activity of a protein-protein interaction.

“Affinity” refers to the strength of the sum total of noncovalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and ligand). The affinity of a molecule X for its partner Y can generally be represented by the dissociation constant (K_{D}). Affinity can be measured by common methods known in the art, including those described herein.

“Complex” or “complexed” as used herein refers to the association of two or more molecules that interact with each other through bonds and/or forces (e.g., Van der Waals, hydrophobic, hydrophilic forces) that are not peptide bonds. In one aspect, a complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

The terms “host cell,” “host cell line,” and “host cell culture” are used interchangeably and refer to cells into which exogenous nucleic acid has been introduced, including the progeny of such cells. Host cells include “transfected cells,” “transformed cells,” and “transformants,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, the host cell is stably transformed with the exogenous nucleic acid. In other aspects, the host cell is transiently transformed with the exogenous nucleic acid.

The terms “Herpes simplex virus 2 (HSV-2) glycoprotein D (gD),” “HSV-2 gD,” and variants thereof, as used herein, refer to a native gD protein from a HSV-2 virus. The term encompasses full-length gD and isolated regions or domains of gD, e.g., the gD ECD. The term also encompasses naturally occurring variants of gD, e.g., splice variants or allelic variants. The amino acid sequence of an exemplary HSV-2 gD is shown in SEQ ID NO: 1. Minor sequence variations, especially conservative amino acid substitutions of gD that do not affect gD function and/or activity, are also contemplated by the invention.

The terms “Herpes simplex virus 2 (HSV-2) glycoprotein G (gG),” “HSV-2 gG,” and variants thereof, as used herein, refer to a native gG protein from a HSV-2 virus. The term encompasses full-length gG and isolated regions or domains of gG, e.g., the gG ECD. The term also encompasses naturally occurring variants of gG, e.g., splice variants or allelic variants. The amino acid sequence of an exemplary HSV-2 gG is shown in SEQ ID NO: 2. Minor sequence variations, especially conservative amino acid substitutions of gG that do not affect gG function and/or activity, are also contemplated by the invention.

The terms “Macacine alphaherpesvirus (MCHV) glycoprotein G (gG),” “MCHV gG,” and variants thereof, as used herein, refer to a native gG protein from a MCHV virus. The term encompasses full-length gG and isolated regions or domains of gG, e.g., the gG ECD. The term also encompasses naturally occurring variants of gG, e.g., splice variants or allelic variants. The amino acid sequence of an exemplary MCHV gG is shown in SEQ ID NO: 3. Minor sequence variations, especially conservative amino acid substitutions of gG that do not affect gG function and/or activity, are also contemplated by the invention.

The terms “human cytomegalovirus (HCMV) UL6 protein,” “HCMV UL6,” and variants thereof, as used herein, refer to a native UL6 protein from a HCMV virus. The term encompasses full-length UL6 and isolated regions or domains of UL6, e.g., the UL6 ECD. The term also encompasses naturally occurring variants of UL6, e.g., splice variants or allelic variants. The amino acid sequence of an exemplary HCMV UL6 is shown in SEQ ID NO: 4. Minor sequence variations, especially conservative amino acid substitutions of UL6 that do not affect UL6 function and/or activity, are also contemplated by the invention.

The terms “human cytomegalovirus (HCMV) UL9 protein,” “HCMV UL9,” and variants thereof, as used herein, refer to a native UL9 protein from a HCMV virus. The term encompasses full-length UL9 and isolated regions or domains of UL9, e.g., the UL9 ECD. The term also encompasses naturally occurring variants of UL9, e.g., splice variants or allelic variants. The amino acid sequence of an exemplary HCMV UL9 is shown in SEQ ID NO: 5. Minor sequence variations, especially conservative amino acid substitutions of UL9 that do not affect UL9 function and/or activity, are also contemplated by the invention.

The terms “human cytomegalovirus (HCMV) UL142 protein,” “HCMV UL142,” and variants thereof, as used herein, refer to a native UL142 protein from a HCMV virus. The term encompasses full-length UL142 and isolated regions or domains of UL142, e.g., the UL142 ECD. The term also encompasses naturally occurring variants of UL142, e.g., splice variants or allelic variants. The amino acid sequence of an exemplary HCMV UL142 is shown in SEQ ID NO: 6. Minor sequence variations, especially conservative amino acid substitutions of UL142 that do not affect UL142 function and/or activity, are also contemplated by the invention.

The terms “human cytomegalovirus (HCMV) UL144 protein,” “HCMV UL144,” and variants thereof, as used herein, refer to a native UL144 protein from a HCMV virus. The term encompasses full-length UL144 and isolated regions or domains of UL144, e.g., the UL144 ECD. The term also encompasses naturally occurring variants of UL144, e.g., splice variants or allelic variants. The amino acid sequence of an exemplary HCMV UL144 is shown in SEQ ID NO: 7. Minor sequence variations, especially conservative amino acid substitutions of UL144 that do not affect UL144 function and/or activity, are also contemplated by the invention.

The terms “human cytomegalovirus (HCMV) RL10 protein,” “HCMV RL10,” and variants thereof, as used herein, refer to a native RL10 protein from a HCMV virus. The term encompasses full-length RL10 and isolated regions or domains of RL10, e.g., the RL10 ECD. The term also encompasses naturally occurring variants of RL10, e.g., splice variants or allelic variants. The amino acid sequence of an exemplary HCMV RL10 is shown in SEQ ID NO: 8. Minor sequence variations, especially conservative amino acid substitutions of RL10 that do not affect RL10 function and/or activity, are also contemplated by the invention.

The terms “Varicella zoster virus (VZV) glycoprotein C,” “VZV gC,” and variants thereof, as used herein, refer to a native gC protein from a VZV virus. The term encompasses full-length gC and isolated regions or domains of gC, e.g., the gC ECD. The term also encompasses naturally occurring variants of gC, e.g., splice variants or allelic variants. The amino acid sequence of an exemplary VZV gC is shown in SEQ ID NO: 9. Minor sequence variations, especially conservative amino acid substitutions of gC that do not affect gC function and/or activity, are also contemplated by the invention.

The terms “Varicella zoster virus (VZV) glycoprotein B,” “VZV gB,” and variants thereof, as used herein, refer to a native gB protein from a VZV virus. The term encompasses full-length gB and isolated regions or domains of gB, e.g., the gB ECD. The term also encompasses naturally occurring variants of gB, e.g., splice variants or allelic variants. The amino acid sequence of an exemplary VZV gB is shown in SEQ ID NO: 10. Minor sequence variations, especially conservative amino acid substitutions of gB that do not affect gB function and/or activity, are also contemplated by the invention.

The terms “Varicella zoster virus (VZV) glycoprotein I,” “VZV gI,” and variants thereof, as used herein, refer to a native gI protein from a VZV virus. The term encompasses full-length gI and isolated regions or domains of gI, e.g., the gI ECD. The term also encompasses naturally occurring variants of gI, e.g., splice variants or allelic variants. The amino acid sequence of an exemplary VZV gI is shown in SEQ ID NO: 11. Minor sequence variations, especially conservative amino acid substitutions of gI that do not affect gI function and/or activity, are also contemplated by the invention.

The terms “human herpesvirus 8 (HHV8) K14,” “HHV8 K14,” and variants thereof, as used herein, refer to a native K14 protein from a HHV8 virus. The term encompasses full-length K14 and isolated regions or domains of K14, e.g., the K14 ECD. The term also encompasses naturally occurring variants of K14, e.g., splice variants or allelic variants. The amino acid sequence of an exemplary HHV8 K14 is shown in SEQ ID NO: 12. Minor sequence variations, especially conservative amino acid substitutions of K14 that do not affect K14 function and/or activity, are also contemplated by the invention.

The terms “human herpesvirus 8 (HHV8) KCP,” “HHV8 KCP,” and variants thereof, as used herein, refer to a native KCP protein from a HHV8 virus. The term encompasses full-length KCP and isolated regions or domains of KCP, e.g., the KCP ECD. The term also encompasses naturally occurring variants of KCP, e.g., splice variants or allelic variants. The amino acid sequence of an exemplary HHV8 KCP is shown in SEQ ID NO: 13. Minor sequence variations, especially conservative amino acid substitutions of KCP that do not affect KCP function and/or activity, are also contemplated by the invention.

The term “antagonist,” as used herein with regard to a protein, refers to a molecule that decreases signal transduction resulting from the interaction of the protein with one or more of its binding partners. The antagonist may result in a decrease in the binding of the protein to one or more of its binding partners relative to binding of the two proteins in the absence of the antagonist. Antagonists may include antibodies, antigen binding fragments thereof, immunoadhesins, fusion proteins, peptides (e.g., multimerized peptides), oligopeptides, inhibitory nucleic acids (e.g., ASOs or siRNAs), and other molecules that decrease signal transduction resulting from the interaction of the protein with one or more of its binding partners.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antigen-binding fragment” or “antibody fragment” refers to a molecule other than an intact antibody that comprises a portion of an intact antibody that binds the antigen to which the intact antibody binds. Examples of antigen-binding fragments include but are not limited to bis-Fabs; Fv; Fab; Fab, Fab′-SH; F(ab′)₂; diabodies; linear antibodies; single-chain antibody molecules (e.g., scFv, scFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The terms “full-length antibody,” “intact antibody,” and “whole antibody” are used herein interchangeably to refer to an antibody having a structure substantially similar to a native antibody structure or having heavy chains that contain an Fc region as defined herein.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

The term “small molecule” refers to any molecule with a molecular weight of about 2000 daltons or less, e.g., about 1000 daltons or less. In some aspects, the small molecule is a small organic molecule.

The term “mimic” or “molecular mimic,” as used herein, refers to a polypeptide having sufficient similarity in conformation and/or binding ability (e.g., secondary structure, tertiary structure) to a given polypeptide or to a portion of said polypeptide to bind to a binding partner of said polypeptide. The mimic may bind the binding partner with equal, less, or greater affinity than the polypeptide it mimics. A molecular mimic may or may not have obvious amino acid sequence similarity to the polypeptide it mimics. A mimic may be naturally occurring or may be engineered. In some aspects, the mimic is a mimic of a member of a binding pair. In yet other aspects, the mimic is a mimic of another protein that binds to a member of the binding pair. In some aspects, the mimic may perform all functions of the mimicked polypeptide. In other aspects, the mimic does not perform all functions of the mimicked polypeptide.

As used herein, the term “conditions permitting the binding” of two or more proteins to each other refers to conditions (e.g., protein concentration, temperature, pH, salt concentration) under which the two or more proteins would interact in the absence of a modulator or a candidate modulator. Conditions permitting binding may differ for individual proteins and may differ between protein-protein interaction assays (e.g., surface plasmon resonance assays, biolayer interferometry assays, enzyme-linked immunosorbent assays (ELISA), extracellular interaction assays, and cell surface interaction assays.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some aspects, the sample is a formalin-fixed and paraffin-embedded (FFPE) tumor tissue sample.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease (e.g., preventing herpesvirus infection, genital herpes, herpes simplex encephalitis, zoonotic MCHV infection, HCMV infection, chicken pox, shingles, Kaposi's sarcoma, HHV-associated multicentric Castleman's disease, primary effusion lymphoma, or KSHV inflammatory cytokine syndrome), reducing or preventing recurrent or secondary infection in a patient having an infection, alleviation of symptoms, diminishment of any direct or indirect pathological consequences of the disease, decreasing the rate of disease progression, amelioration or palliation of the disease state, and remission or improved prognosis.

The “pathology” of a disease or condition includes all phenomena that compromise the well-being of the patient.

### II. METHODS OF IDENTIFYING MODULATORS OF HERPESVIRUS SURFACE PROTEIN INTERACTIONS

### A. Methods of Identifying Modulators

In some aspects, the disclosure features a method of identifying a modulator of the interaction between a protein of Table 1 and a protein of Table 2, the method comprising: (a) providing a candidate modulator; (b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2. Increased or decreased binding may be assessed using, e.g., surface plasmon resonance, biolayer interferometry, or an enzyme-linked immunosorbent assay (ELISA).

TABLE 1
Herpesvirus proteins

Herpes simplex virus 2 (HSV-2) glycoprotein G (gG)
HSV-2 glycoprotein D (gD)
Macacine alphaherpesvirus (MCHV) glycoprotein G (gG)
HCMV UL6
HCMV UL9
HCMV UL142
HCMV UL144
HCMV RL10
Varicella zoster virus (VZV) glycoprotein C (gC)
VZV glycoprotein B (gB)
VZV glycoprotein I (gl)
Human herpesvirus 8 (HHV8) K14 protein
HHV8 KCP protein

TABLE 2
Human single-pass transmembrane (STM) proteins
CSPG5
PRRG2
UNC5D
PLB1
PILRA
VEGFR2
MERTK
(MER)
PDGFRa
KIRREL2
LILRB5
ULBP1
KIR2DL3
KIR2DS1
KIR2DS2
KIR2DS4
KIR2DS5
KIR2DL1
KIR3DL1
KLRAP1
SGCA
ICAM1
MUSK
HAVCR1
MOG
KIAA0319L
KLRAP1
LILRB1
CLEC4G
FLRT1
FLRT2
FLRT3

TABLE 3
Protein-protein interactions

| Protein A | Protein B |
|---|---|
| HSV-2 gG | CSPG5 |
| | PRRG2 |
| | UNC5D |
| HSV-2 gD | PLB1 |
| MCHV gG | PILRA |
| HCMV UL6 | VEGFR2 |
| | MERTK |
| | (MER) |
| | PDGFRa |
| | KIRREL2 |
| HCMV UL9 | LILRB5 |
| | ULBP1 |
| | KIR2DL3 |
| | KIR2DS1 |
| | KIR2DS2 |
| | KIR2DS4 |
| | KIR2DS5 |
| | KIR2DL1 |
| | KIR3DL1 |
| HCMV | PRRG2 |
| UL142 | |
| HCMV | KLRAP1 |
| UL144 | |
| HCMV RL10 | SGCA |
| VZV gC | ICAM1 |
| VZV gB | MUSK |
| | HAVCR1 |
| VZV gl | MOG |
| | KIAA0319L |
| HHV8 K14 | KLRAP1 |
| HHV8 KCP | LILRB1 |
| | CLEC4G |
| | FLRT1 |
| | FLRT2 |
| | FLRT3 |

In some aspects, the candidate modulator is identified as a modulator if the increase in binding is at least 40%. In some aspects, the increase in binding is at least 5%, at least 10%, at least 20%, at least 30%, at least 40%, at least 50%, at least 60%, at least 70%, at least 80%, at least 90%, at least 100%, or more than 100% (e.g., 5%-15%, 15%-25%, 25%-35%, 35%-45%, 45%-55%, 55%-65%, 65%-75%, 75%-85%, 85%-95%, 95%-100%, or more than 100%). In some aspects, the increase in binding is at least 70%.

In some aspects, the candidate modulator is identified as a modulator if the decrease in binding is at least 40%. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 70%.

In some aspects, the disclosure features a method of identifying a modulator of a downstream activity of a protein of Table 1, the method comprising (a) providing a candidate modulator; (b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1.

In some aspects, the disclosure features a method of identifying a modulator of a downstream activity of a protein of Table 2, the method comprising (a) providing a candidate modulator; (b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and (c) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

In some aspects, the modulator is an inhibitor of the downstream activity of the protein of Table 1 or the protein of Table 2. In some aspects, the change in the downstream activity is a decrease in the amount, strength, or duration of the downstream activity. In some aspects, the downstream activity of the protein of Table 1 or the protein of Table 2 is infection of a cell by a member of the viral family Herpesviridae. In some aspects, infection is decreased in the presence of the modulator, e.g., decreased by at least 5%, 10%, 15%, 20%, 25%, 30%, 35%, 40%, 45%, 50%, 55%, 60%, 65%, 70%, 75%, 80%, 85%, 90%, 95%, 98%, or 99% or decreased by 100% (i.e., abolished), e.g., decreased by 5%-15%, 15%-25%, 25%-35%, 35%-45%, 45%-55%, 55%-65%, 65%-75%, 75%-85%, 85%-95%, or 95%-100%, e.g., as measured in a viral infection assay (e.g., as described in Cantuti-Castelvetri et al., Science, DOI: 10.1126/science.abd2985, 2020 or a viral entry assay). In some aspects, infection is decreased by at least 40% in the presence of the modulator.

In some aspects, the modulator is an activator of the downstream activity of the protein of Table 1 or the protein of Table 2. In some aspects, the change in the downstream activity is an increase in the amount, strength, or duration of the downstream activity.

In some aspects, the modulator is an antibody or antigen-binding fragment thereof that binds the protein of Table 1. In some aspects, the modulator is an antibody or antigen-binding fragment thereof that binds the protein of Table 2.

In another aspect, the disclosure features a method of identifying a modulator of the interaction between the HCMV UL6 protein and MERTK or VEGFR2, the method comprising (a) providing a candidate modulator; (b) contacting the HCMV UL6 protein with MERTK or VEGFR2 in the presence or absence of the candidate modulator under conditions permitting the binding of the HCMV UL6 protein to MERTK or VEGFR2; and (c) measuring the binding of the HCMV UL6 protein to MERTK or VEGFR2, wherein an increase or decrease in binding in the presence of the candidate modulator relative to binding in the absence of the candidate modulator identifies the candidate modulator as a modulator of the interaction between the HCMV UL6 protein and MERTK or VEGFR2. In some aspects, the method is a method of identifying a modulator of the interaction between the HCMV UL6 protein and MERTK, and the method comprises contacting the HCMV UL6 protein with MERTK. In other aspects, the method is a method of identifying a modulator of the interaction between the HCMV UL6 protein and VEGFR2, and the method comprises contacting the HCMV UL6 protein with VEGFR2.

In another aspect, the disclosure features a method of identifying a modulator of a downstream activity of the HCMV UL6 protein, the method comprising (a) providing a candidate modulator; (b) contacting the HCMV UL6 protein with MERTK or VEGFR2 in the presence or absence of the candidate modulator under conditions permitting the binding of the HCMV UL6 protein to MERTK or VEGFR2; and (c) measuring a downstream activity of the HCMV UL6 protein, wherein a change in the downstream activity in the presence of the candidate modulator relative to the downstream activity in the absence of the candidate modulator identifies the candidate modulator as a modulator of the downstream activity of the HCMV UL6 protein. In some aspects, the method comprises contacting the HCMV UL6 protein with MERTK. In other aspects, the method comprises contacting the HCMV UL6 protein with VEGFR2.

In another aspect, the disclosure features a method of identifying a modulator of a downstream activity of MERTK or VEGFR2, the method comprising (a) providing a candidate modulator; (b) contacting MERTK or VEGFR2 with the HCMV UL6 protein in the presence or absence of the candidate modulator under conditions permitting the binding of MERTK or VEGFR2 to the HCMV UL6 protein; and (c) measuring a downstream activity of MERTK or VEGFR2, wherein a change in the downstream activity in the presence of the candidate modulator relative to the downstream activity in the absence of the candidate modulator identifies the candidate modulator as a modulator of the downstream activity of MERTK or VEGFR2. In some aspects, the method is a method of identifying a modulator of a downstream activity of MERTK, and the method comprises contacting MERTK with the HCMV UL6 protein. In other aspects, the method is a method of identifying a modulator of a downstream activity of VEGFR2, and the method comprises contacting VEGFR2 with the HCMV UL6 protein.

In some aspects, the candidate modulator is identified as a modulator if the increase in binding is at least 40%. In some aspects, the increase in binding is at least 5%, at least 10%, at least 20%, at least 30%, at least 40%, at least 50%, at least 60%, at least 70%, at least 80%, at least 90%, at least 100%, or more than 100% (e.g., 5%-15%, 15%-25%, 25%-35%, 35%-45%, 45%-55%, 55%-65%, 65%-75%, 75%-85%, 85%-95%, 95%-100%, or more than 100%). In some aspects, the increase in binding is at least 70%.

In some aspects, the candidate modulator is identified as a modulator if the decrease in binding is at least 40%. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 70%.

In some aspects, the increase or decrease in binding is at least 70%, as measured by a surface plasmon resonance (SPR) assay, a BLI assay, or an enzyme-linked immunosorbent assay (ELISA).

In some aspects, the modulator is an inhibitor of the downstream activity of the HCMV UL6 protein or MERTK or VEGFR2. In some aspects, the change in the downstream activity is a decrease in the amount, strength, or duration of the downstream activity. In some aspects, the downstream activity of the HCMV UL6 protein or MERTK or VEGFR2 is infection of a cell by HCMV. In some aspects, infection is decreased in the presence of the modulator, e.g., decreased by at least 5%, 10%, 15%, 20%, 25%, 30%, 35%, 40%, 45%, 50%, 55%, 60%, 65%, 70%, 75%, 80%, 85%, 90%, 95%, 98%, or 99% or decreased by 100% (i.e., abolished), e.g., decreased by 5%-15%, 15%-25%, 25%-35%, 35%-45%, 45%-55%, 55%-65%, 65%-75%, 75%-85%, 85%-95%, or 95%-100%, e.g., as measured in a viral infection assay (e.g., as described in Cantuti-Castelvetri et al., Science, DOI: 10.1126/science.abd2985, 2020 or a viral entry assay). In some aspects, infection is decreased by at least 40% in the presence of the modulator.

In some aspects, the downstream activity of the HCMV UL6 protein or MERTK or VEGFR2 (e.g., the downstream activity of the HCMV UL6 protein and/or MERTK) is angiogenesis. In some aspects, angiogenesis (e.g., angiogenesis associated with HCMV infection) is decreased in the presence of the modulator, e.g., decreased by at least 5%, 10%, 15%, 20%, 25%, 30%, 35%, 40%, 45%, 50%, 55%, 60%, 65%, 70%, 75%, 80%, 85%, 90%, 95%, 98%, or 99% or decreased by 100% (i.e., abolished), e.g., decreased by 5%-15%, 15%-25%, 25%-35%, 35%-45%, 45%-55%, 55%-65%, 65%-75%, 75%-85%, 85%-95%, or 95%-100%, e.g., as measured in a tube formation assay (e.g., as described in Example 3). In some aspects, angiogenesis is decreased by at least 40% in the presence of the modulator, e.g., as measured in a tube formation assay. In some aspects, the occurrence or severity of a HCMV-associated vasculopathy (e.g., atherosclerosis, transplant vascular sclerosis, or glioblastoma) is decreased in the presence of the modulator, e.g., decreased by at least 5%, 10%, 15%, 20%, 25%, 30%, 35%, 40%, 45%, 50%, 55%, 60%, 65%, 70%, 75%, 80%, 85%, 90%, 95%, 98%, or 99% or decreased by 100% (i.e., abolished), e.g., decreased by 5%-15%, 15%-25%, 25%-35%, 35%-45%, 45%-55%, 55%-65%, 65%-75%, 75%-85%, 85%-95%, or 95%-100%.

In some aspects, the downstream activity of the HCMV UL6 protein or MERTK is phosphorylation of MERTK. In some aspects, phosphorylation of MERTK is decreased in the presence of the modulator, e.g., decreased by at least 5%, 10%, 15%, 20%, 25%, 30%, 35%, 40%, 45%, 50%, 55%, 60%, 65%, 70%, 75%, 80%, 85%, 90%, 95%, 98%, or 99% or decreased by 100% (i.e., abolished), e.g., decreased by 5%-15%, 15%-25%, 25%-35%, 35%-45%, 45%-55%, 55%-65%, 65%-75%, 75%-85%, 85%-95%, or 95%-100%, e.g., as measured using a Western blot (e.g., a Western blot using an antibody that detects phosphoMER (pMer)) or ELISA. In some aspects, phosphorylation of MERTK is decreased by at least 40% in the presence of the modulator, e.g., as measured using a Western blot or ELISA. In some aspects, phosphorylation of MERTK is decreased in the presence of the modulator, e.g., decreased by at least 5%, 10%, 15%, 20%, 25%, 30%, 35%, 40%, 45%, 50%, 55%, 60%, 65%, 70%, 75%, 80%, 85%, 90%, 95%, 98%, or 99% or decreased by 100% (i.e., abolished), e.g., decreased by 5%-15%, 15%-25%, 25%-35%, 35%-45%, 45%-55%, 55%-65%, 65%-75%, 75%-85%, 85%-95%, or 95%-100%, e.g., as measured using a Western blot or ELISA.

In some aspects, the modulator is an activator of the downstream activity of the HCMV UL6 protein or MERTK or VEGFR2. In some aspects, the change in the downstream activity is an increase in the amount, strength, or duration of the downstream activity.

In some aspects, the antibody or antigen-binding fragment thereof binds the HCMV UL6 protein. In some aspects, the antibody or antigen-binding fragment thereof binds MERTK. In some aspects, the antibody or antigen-binding fragment thereof binds VEGFR2.

### B. Modulators

In some aspects, the modulator or candidate modulator of the interaction between the protein of Table 1 and the protein of Table 2 is a small molecule, an antibody or antigen-binding fragment thereof, a peptide, a mimic, or an inhibitory nucleic acid (e.g., an antisense oligonucleotide (ASO) or an siRNA). In some aspects, the antigen-binding fragment is a bis-Fab, an Fv, a Fab, a Fab′-SH, a F(ab′)₂, a diabody, a linear antibody, an scFv, an scFab, a VH domain, or a VHH domain. Exemplary modulators are further described in Section III herein.

### C. Assays for Modulation of Protein-Protein Interactions

In some aspects, the binding of the protein of Table 1 and the protein of Table 2 in the presence or absence of the candidate modulator is assessed in an assay for protein-protein interaction. Modulation of the interaction between the protein of Table 1 and the protein of Table 2 may be identified as an increase in protein-protein interaction in the presence of the modulator compared to protein-protein interaction in the absence of the modulator, e.g., an increase of 5%, 10%, 15%, 20%, 25%, 30%, 35%, 40%, 45%, 50%, 55%, 60%, 65%, 70%, 80%, 90%, 95%, 100%, or more than 100% (e.g., 5%-15%, 15%-25%, 25%-35%, 35%-45%, 45%-55%, 55%-65%, 65%-75%, 75%-85%, 85%-95%, 95%-100%, or more than 100%) in protein-protein interaction. Alternatively, modulation may be identified as a decrease in protein-protein interaction in the presence of the modulator compared to protein-protein interaction in the absence of the modulator, e.g., an decrease of 5%, 10%, 15%, 20%, 25%, 30%, 35%, 40%, 45%, 50%, 55%, 60%, 65%, 70%, 80%, 90%, 95%, or 100% (e.g., 5%-15%, 15%-25%, 25%-35%, 35%-45%, 45%-55%, 55%-65%, 65%-75%, 75%-85%, 85%-95%, or 95%-100%) in protein-protein interaction. The assay for protein-protein interaction may be, e.g., an SPR assay, a biolayer interferometry (BLI) assay, an enzyme-linked immunosorbent assay (ELISA), an extracellular interaction assay, or a cell surface interaction assay.

### Cell Surface Interaction Assays

In some aspects of the invention, the protein-protein interaction assay is a cell surface interaction assay. In this type of assay, one or more prey proteins are expressed as extracellular domain (ECD) fusion proteins on the cell surface and are tested for interaction with one or more bait proteins expressed as a soluble construct using, e.g., a fluorescent assay wherein the bait protein comprises a fluorescent tag.

In some aspects, the prey protein or prey proteins comprise one or more fusion proteins in which the extracellular domain (ECD) of a prey protein of interest is conjugated (e.g., fused) to one or more additional moieties (e.g., a glycosylphosphatidylinositol (GPI)-gD (gDGPI) tag) such that the prey fusion protein is expressed on the cell surface.

In some aspects in which the polypeptide comprises an extracellular domain, a tag, and an anchor, the anchor is capable of tethering the extracellular domain to the surface of a plasma membrane of a cell. In some aspects, the anchor is a glycosylphosphatidyl-inositol (GPI) polypeptide. In some aspects, the anchor is a moiety used in protein lipidation, e.g., a moiety used in cysteine palmitoylation, glycine myristoylation, lysine fatty-acylation, cholesterol esterification, cysteine prenylation, or serine fatty-acylation.

In some aspects, the tag can be directly or indirectly visualized, or otherwise detected. For example, the tag may comprise a moiety that can be detected using an antibody or an antibody fragment, e.g., may be a glycoprotein D (gD) polypeptide. In some aspects, the tag comprises a fluorescent protein.

The bait protein may be conjugated to one or more additional moieties such that the bait fusion protein is soluble. The additional moiety or moieties may also increase the avidity of the bait fusion protein for the prey protein, e.g., by multimerizing the bait protein. Increasing avidity may increase the detection of low-affinity interactions. In some aspects, the additional moiety causes tetramerization of the bait protein.

In some aspects, the bait fusion protein comprises an Avi tag, a cleavage sequence (e.g., a TEV cleavage sequence), and an Fc tag, such that the Fc tag can be cleaved from the protein upon addition of the enzyme TEV protease. To prepare this protein for a cell surface interaction assay, the Fc tag is cleaved, the Avi tag is biotinylated, and the biotinylated bait fusion protein is conjugated to a fluorescent streptavidin (SA), e.g., a streptavidin conjugated to allophycocyanin (APC), to form a tetramerized bait fusion protein detectable in a fluorescence assay.

The prey fusion protein may be expressed (e.g., transfected, e.g., transiently transfected) in a cell. The cell may be a human cell, e.g., a COS7 cell. Transfected cells may be placed in a well, e.g., a well in a 384-well plate.

The bait fusion protein may be expressed (e.g., transfected, e.g., transiently transfected) in a cell, e.g., a mammalian cell. Bait fusion proteins may be purified using standard protocols, e.g., as described in Ramani et al., Anal Biochem, 420: 127-138, 2012.

To perform the protein-protein interaction assay, a solution comprising the bait protein (e.g., the purified bait fusion protein conjugated to fluorescent SA) may be added to one or more wells containing cells expressing a prey protein (e.g., to one or more wells of a 384-well plate). The assay may then be incubated and washed one or more times to remove non-bound bait protein. The cells may then be fixed, e.g., with 4% paraformaldehyde, to preserve protein-protein interactions.

In some aspects, detecting an interaction comprises detecting a signal, e.g., a fluorescent signal, at a location on the solid surface that is above a threshold level (e.g., a signal indicating the presence of a query protein at the location, e.g., a signal from a moiety comprised by the bait fusion protein (e.g., multimerized query protein)). The signal may be directly or indirectly visualizable or otherwise detectable. In some aspects, the detecting is semi-automated or automated. The interaction may be a transient interaction and/or a low-affinity interaction, e.g., a micromolar-affinity interaction.

In aspects in which the bait fusion protein (e.g., a multimerized query protein) comprises a fluorescent SA, interaction between the bait fusion protein and the prey fusion protein may be detected by fluorescence microscopy. Relatively high fluorescence indicates that the bait fusion protein is present, i.e., that the bait fusion protein and the prey fusion protein interact.

### Extracellular Interaction Assays

In some aspects of the invention, the protein-protein interaction assay is an extracellular interaction assay, e.g., an avidity-based extracellular interaction screen (AVEXIS) (Bushell et al., Genome Res, 18: 622-630, 2008; Martinez-Martin et al., J Immunol Res, 2197615, 2017).

### SPR Assays for Modulation of Protein-Protein Interaction

In some aspects, the assay for protein-protein interaction is a surface plasmon resonance (SPR) assay. In some aspects, SPR assays are used to confirm or validate assays detected in an extracellular interaction assay or a cell surface interaction assay, e.g., a high-throughput extracellular interaction screen or a high-throughput cell surface interaction screen.

In some aspects, a prey protein is expressed as a fusion protein comprising the extracellular domain (ECD) of the protein conjugated to an additional moiety, e.g., an Fc tag. The prey fusion protein may be purified. The prey protein may be immobilized on a sensor chip, e.g. a GLC or CM5 sensor chip, by amine coupling.

The bait protein may be provided in a soluble form, e.g., as a protein domain fused to a soluble tag. The bait fusion protein may be purified.

In some aspects, modulation of the binding of the protein of Table 1 and the protein of Table 2 is measured as a difference in SPR signal response units (RU) in the presence compared to the absence of the modulator.

### BLI Assays for Modulation of Protein-Protein Interaction

In some aspects, the assay for protein-protein interaction is a biolayer interferometry (BLI) assay. In some aspects, the BLI assay is performed using isolated ECDs, e.g., isolated ECDs as described herein. In some aspects, modulation of the binding of the protein of Table 1 and the protein of Table 2 is measured as a difference in wavelength shift (Δλ) measured at a biosensor tip in the presence compared to the absence of the modulator.

### ELISA for Modulation of Protein-Protein Interaction

In some aspects, the assay for protein-protein interaction is an enzyme-linked immunosorbent assay (ELISA). In some aspects, a first protein is bound to a plate (e.g., directly bound to a plate or bound to a plate via an affinity tag recognized by an antibody bound to a plate) and a second protein is provided in a soluble form, e.g., as an isolated ECD as described herein. An interaction between the first protein and the second protein may be detected by providing an antibody that binds to the second protein or to an affinity tag thereof, wherein the antibody can be detected, e.g., visualized, in an assay for presence of the antibody.

### Other Assays for Modulation of Protein-Protein Interaction

In some aspects, the assay is an isothermal titration calorimetry (ITC) assay, an assay comprising immunoprecipitation, or an assay comprising an ALPHASCREEN™ technology.

In some aspects of the above assays, the candidate modulator is provided to a cell (e.g., a mammalian cell), to cell culture media, to conditioned media, and/or to a purified form of the protein of Table 1 and/or the protein of Table 2. In some aspects, the candidate modulator is provided at a concentration of at least 0.1 nM, 0.5 nM, 1 nM, 10 nM, 50 nM, 100 nM, 250 nM, 500 nM, 750 nM, 1 μM, 2 μM, 3 μM, 5 μM, or 10 μM. In some aspects, the candidate modulator is provided at a concentration of between 0.1 nM and 10 μM. In some aspects, the candidate modulator is provided in a solution, e.g., in a soluble form.

### Thresholds for Identification of Modulators

In some aspects, the candidate modulator is identified as a modulator if the increase in binding is at least 50%. In some aspects, the increase in binding is at least 5%, at least 10%, at least 20%, at least 30%, at least 40%, at least 50%, at least 60%, at least 70%, at least 80%, at least 90%, at least 100%, or more than 100% (e.g., 5%-15%, 15%-25%, 25%-35%, 35%-45%, 45%-55%, 55%-65%, 65%-75%, 75%-85%, 85%-95%, 95%-100%, or more than 100%). In some aspects, the increase in binding is at least 50%.

In some aspects, the candidate modulator is identified as a modulator if the decrease in binding is at least 50%. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 50%.

Exemplary methods for identifying modulators of protein-protein interactions, as well as agents that may modulate such interactions, are described in PCT/US2020/025471, which is hereby incorporated by reference.

### III. MODULATORS OF PROTEIN-PROTEIN INTERACTIONS

### A. Small Molecules

In some aspects, the modulator or candidate modulator is a small molecule. Small molecules are molecules other than binding polypeptides or antibodies as defined herein that may bind, preferably specifically, to a protein of Table 1 or a protein of Table 2. Binding small molecules may be identified and chemically synthesized using known methodology (see, e.g., PCT Publication Nos. WO00/00823 and WO00/39585). Binding small molecules are usually less than about 2000 daltons in size (e.g., less than about 2000, 1500, 750, 500, 250 or 200 daltons in size), wherein such organic small molecules that are capable of binding, preferably specifically, to a polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). Binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some aspects, the binding of the protein of Table 1 and the protein of Table 2 is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the small molecule. In some aspects, the binding of the protein of Table 1 and the protein of Table 2 is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the small molecule. In some aspects, a downstream activity (e.g., viral infection of a cell) of the protein of Table 1 or the protein of Table 2 is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the small molecule.

### B. Antibodies and Antigen-Binding Fragments

In some aspects, the modulator or candidate modulator is an antibody or an antigen-binding fragment thereof binding the protein of Table 1 or the protein of Table 2. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the modulator is an antibody or antigen-binding fragment thereof that binds the protein of Table 1. In some aspects, the antibody or antigen-binding fragment thereof that binds the protein of Table 1 blocks the interaction of the protein of Table 1 with the protein of Table 2.

In some aspects, the modulator is an antibody or antigen-binding fragment thereof that binds the protein of Table 2, e.g., binds to an epitope of the protein of Table 2 that interacts with the protein of Table 1. In some aspects, the antibody or antigen-binding fragment thereof that binds the protein of Table 2 blocks the interaction of the protein of Table 2 with the protein of Table 1.

In some aspects, the modulator is a multispecific antibody, e.g., a bispecific antibody.

In some aspects, the binding of the protein of Table 1 and the protein of Table 2 is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the antibody or antigen-binding fragment. In some aspects, the binding of the protein of Table 1 and the protein of Table 2 is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the antibody or antigen-binding fragment. In some aspects, a downstream activity (e.g., viral infection of a cell) of a protein of Table 1 or a protein of Table 2 is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the antibody or antigen-binding fragment.

### C. Peptides

In some aspects, the modulator or candidate modulator is a peptide that binds the protein of Table 1 or the protein of Table 2. The peptide may be the peptide may be naturally occurring or may be engineered. In some aspects, the peptide is a fragment of the protein of Table 1, the protein of Table 2, or another protein that binds to the protein of Table 1 or the protein of Table 2. The peptide may bind the binding partner with equal, less, or greater affinity than the full-length protein. In some aspects, the peptide performs all functions of the full-length protein. In other aspects, the peptide does not perform all functions of the full-length protein.

In some aspects, the binding of the protein of Table 1 or the protein of Table 2 is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the peptide. In some aspects, the binding of the protein of Table 1 and the protein of Table 2 is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the peptide. In some aspects, a downstream activity of the protein of Table 1 or the protein of Table 2 (e.g., viral infection of a cell) is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the peptide.

### D. Mimics

In some aspects, the modulator or candidate modulator is a mimic, e.g., a molecular mimic, that binds to a protein of Table 1 or a protein of Table 2. The mimic may be a molecular mimic of the protein of Table 1 or the protein of Table 2, or another protein that binds to the protein of Table 1 or the protein of Table 2. In some aspects, the mimic may perform all functions of the mimicked polypeptide. In other aspects, the mimic does not perform all functions of the mimicked polypeptide.

In some aspects, the binding of the protein of Table 1 and the protein of Table 2 is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the mimic. In some aspects, the binding of the protein of Table 1 and the protein of Table 2 is increased (e.g., increased by 5%, 10%, 20%, 30%, 40%, 50%, 60%, 70%, 80%, 90%, 100%, or more than 100%, e.g., increased by 5%-15%, 15%-25%, 25%-35%, 35%-45%, 45%-55%, 55%-65%, 65%-75%, 75%-85%, 85%-95%, 95%-100%, or more than 100%) in the presence of the mimic. In some aspects, a downstream activity of the protein of Table 1 or the protein of Table 2 (e.g., viral infection of a cell) is decreased (e.g., decreased by 5%, 10%, 20%, 30%, 40%, 50%, 60%, 70%, 80%, 90%, or 100%, e.g., decreased by 5%-15%, 15%-25%, 25%-35%, 35%-45%, 45%-55%, 55%-65%, 65%-75%, 75%-85%, 85%-95%, or 95%-100%) in the presence of the mimic.

### IV. METHODS OF TREATING OR PREVENTING HERPESVIRUS INFECTIONS

### A. Methods of Treating Individuals Having HSV-2 Infections

In some aspects, the disclosure features a method of treating an individual having a herpes simplex virus 2 (HSV-2) infection or preventing a HSV-2 infection in an individual comprising administering to the individual an effective amount of a CSPG5 antagonist, a PRRG2 antagonist, a UNC5D antagonist, or a PLB1 antagonist.

In some aspects, the disclosure features a method of decreasing HSV-2 infection in an individual comprising administering to the individual an effective amount of a CSPG5 antagonist, a PRRG2 antagonist, a UNC5D antagonist, or a PLB1 antagonist.

In some aspects, the disclosure features a method of reducing HSV-2 attachment to a cell of an individual comprising administering to the individual an effective amount of a CSPG5 antagonist, a PRRG2 antagonist, a UNC5D antagonist, or a PLB1 antagonist. In some aspects, the administering comprises contacting the cell of the individual with an effective amount of a CSPG5 antagonist, a PRRG2 antagonist, a UNC5D antagonist, or a PLB1 antagonist.

In some aspects, (a) the CSPG5 antagonist results in a decrease in the binding of CSPG5 and the HSV-2 glycoprotein G (gG) protein relative to binding of the two proteins in the absence of the antagonist; (b) the PRRG2 antagonist results in a decrease in the binding of PRRG2 and the HSV-2 gG protein relative to binding of the two proteins in the absence of the antagonist; (c) the UNC5D antagonist results in a decrease in the binding of UNC5D and the HSV-2 gG protein relative to binding of the two proteins in the absence of the antagonist; or (d) the PLB1 antagonist results in a decrease in the binding of PLB1 and the HSV-2 gD protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the CSPG5 antagonist, PRRG2 antagonist, UNC5D antagonist, or PLB1 antagonist reduces the extent and/or severity of HSV-2 infection of the individual relative to infection in the absence of the CSPG5 antagonist, PRRG2 antagonist, UNC5D antagonist, or PLB1 antagonist, respectively.

In some aspects, the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the CSPG5 antagonist, PRRG2 antagonist, UNC5D antagonist, or PLB1 antagonist is a peptide.

In some aspects, the CSPG5 antagonist, PRRG2 antagonist, UNC5D antagonist, or PLB1 antagonist is an antibody or antigen-binding fragment thereof. In some aspects, (a) the antibody or antigen-binding fragment thereof binds the HSV-2 gG protein and inhibits its binding to CSPG5, PRRG2, and/or UNC5D; or (b) the antibody or antigen-binding fragment thereof binds the HSV-2 gD protein and inhibits its binding to PLB1. In some aspects, the antibody or antigen-binding fragment thereof binds CSPG5, PRRG2, UNC5D, or PLB1. In some aspects, (a) the antibody or antigen-binding fragment thereof inhibits the binding of CSPG5, PRRG2, or UNC5D to the HSV-2 gG protein; or (b) the antibody or antigen-binding fragment thereof inhibits the binding of PLB1 to the HSV-2 gD protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has genital herpes or herpes simplex encephalitis. In some aspects, the individual is a human.

### B. Methods of Treating Individuals Having MCHV Infections

In some aspects, the disclosure features a method of treating an individual having a macacine alphaherpesvirus (MCHV) infection or preventing a MCHV infection in an individual comprising administering to the individual an effective amount of a PILRA antagonist.

In some aspects, the disclosure features a method of decreasing MCHV infection in an individual comprising administering to the individual an effective amount of a PILRA antagonist.

In some aspects, the disclosure features a method of reducing MCHV attachment to a cell of an individual comprising administering to the individual an effective amount of a PILRA antagonist. In some aspects, the administering comprises contacting the cell of the individual with an effective amount of a PILRA antagonist.

In some aspects, the PILRA antagonist results in a decrease in the binding of PILRA and the MCHV glycoprotein G (gG) protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the PILRA antagonist reduces the extent and/or severity of MCHV infection of the individual relative to infection in the absence of the PILRA antagonist.

In some aspects, the PILRA antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the PILRA antagonist is a peptide.

In some aspects, the PILRA antagonist is an antibody or antigen-binding fragment thereof. In some aspects, the antibody or antigen-binding fragment thereof binds the MCHV gG protein and inhibits its binding to PILRA. In some aspects, the antibody or antigen-binding fragment thereof binds the MCHV gG protein and inhibits its binding to PILRA. In some aspects, the antibody or antigen-binding fragment thereof inhibits the binding of PILRA to the MCHV gG protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has a zoonotic MCHV infection, e.g., an MCHV infection transmitted by a macaque monkey to a human. In some aspects, the individual is a human.

### C. Methods of Treating Individuals Having HCMV Infections

In some aspects, the disclosure features a method of treating an individual having a human cytomegalovirus (HCMV) infection or preventing a HCMV infection in an individual comprising administering to the individual an effective amount of a VEGFR2 antagonist, a MERTK (MER) antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, a or a SGCA antagonist. In some aspects, the disclosure features a method of treating an individual having a HCMV infection or preventing a HCMV infection in an individual comprising administering to the individual an effective amount of a VEGFR2 antagonist. In some aspects, the disclosure features a method of treating an individual having a HCMV infection or preventing a HCMV infection in an individual comprising administering to the individual an effective amount of a MERTK antagonist.

In some aspects, the disclosure features a method of decreasing HCMV infection in an individual comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist. In some aspects, the disclosure features a method of decreasing HCMV infection in an individual comprising administering to the individual an effective amount of a VEGFR2 antagonist. In some aspects, the disclosure features a method of decreasing HCMV infection in an individual comprising administering to the individual an effective amount of a MERTK antagonist.

In some aspects, the disclosure features a method of reducing HCMV attachment to a cell of an individual comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist. In some aspects, the administering comprises contacting the cell of the individual with an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist. In some aspects, the disclosure features a method of reducing HCMV attachment to a cell of an individual comprising administering to the individual an effective amount of a VEGFR2 antagonist. In some aspects, the disclosure features a method of reducing HCMV attachment to a cell of an individual comprising administering to the individual an effective amount of a MERTK antagonist.

In some aspects, (a) the VEGFR2 antagonist results in a decrease in the binding of VEGFR2 and the HCMV UL6 protein relative to binding of the two proteins in the absence of the antagonist; (b) the MERTK antagonist results in a decrease in the binding of MERTK and the HCMV UL6 protein relative to binding of the two proteins in the absence of the antagonist; (c) the PDGFRa antagonist results in a decrease in the binding of PDGFRa and the HCMV UL6 protein relative to binding of the two proteins in the absence of the antagonist; (d) the KIRREL2 antagonist results in a decrease in the binding of KIRREL2 and the HCMV UL6 protein relative to binding of the two proteins in the absence of the antagonist; (e) the LILRB5 antagonist results in a decrease in the binding of LILRB5 and the HCMV UL9 protein relative to binding of the two proteins in the absence of the antagonist; (f) the ULBP1 antagonist results in a decrease in the binding of ULBP1 and the HCMV UL9 protein relative to binding of the two proteins in the absence of the antagonist; (g) the KIR2DL3 antagonist results in a decrease in the binding of KIR2DL3 and the HCMV UL9 protein relative to binding of the two proteins in the absence of the antagonist; (h) the KIR2DS1 antagonist results in a decrease in the binding of KIR2DS1 and the HCMV UL9 protein relative to binding of the two proteins in the absence of the antagonist; (i) the KIR2DS2 antagonist results in a decrease in the binding of KIR2DS2 and the HCMV UL9 protein relative to binding of the two proteins in the absence of the antagonist; (j) the KIR2DS4 antagonist results in a decrease in the binding of KIR2DS4 and the HCMV UL9 protein relative to binding of the two proteins in the absence of the antagonist; (k) the KIR2DS5 antagonist results in a decrease in the binding of KIR2DS5 and the HCMV UL9 protein relative to binding of the two proteins in the absence of the antagonist; (l) the KIR2DL1 antagonist results in a decrease in the binding of KIR2DL1 and the HCMV UL9 protein relative to binding of the two proteins in the absence of the antagonist; (m) the KIR3DL1 antagonist results in a decrease in the binding of KIR3DL1 and the HCMV UL9 protein relative to binding of the two proteins in the absence of the antagonist; (n) the PRRG2 antagonist results in a decrease in the binding of PRRG2 and the HCMV UL142 protein relative to binding of the two proteins in the absence of the antagonist; (o) the KLRAP1 antagonist results in a decrease in the binding of KLRAP1 and the HCMV UL144 protein relative to binding of the two proteins in the absence of the antagonist; or (p) the SGCA antagonist results in a decrease in the binding of SGCA and the HCMV RL10 protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist reduces the extent and/or severity of HCMV infection of the individual relative to infection in the absence of the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist, respectively. In some aspects, the VEGFR2 antagonist reduces the extent and/or severity of HCMV infection of the individual relative to infection in the absence of the VEGFR2 antagonist. In some aspects, the MERTK antagonist, reduces the extent and/or severity of HCMV infection of the individual relative to infection in the absence of the MERTK antagonist.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof. In some aspects, (a) the antibody or antigen-binding fragment thereof binds the HCMV UL6 protein and inhibits its binding to VEGFR2, MERTK, PDGFRa, and/or KIRREL2; (b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1; (c) the antibody or antigen-binding fragment thereof binds the HCMV UL142 protein and inhibits its binding to PRRG2; (d) the antibody or antigen-binding fragment thereof binds the HCMV UL144 protein and inhibits its binding to KLRAP1; or (e) the antibody or antigen-binding fragment thereof binds the HCMV RL10 protein and inhibits its binding to SGCA. In some aspects, the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA. In some aspects, (a) the antibody or antigen-binding fragment thereof inhibits the binding of VEGFR2, MERTK, PDGFRa, or KIRREL2 to the HCMV UL6 protein; (b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein; (c) the antibody or antigen-binding fragment thereof inhibits the binding of PRRG2 to the HCMV UL142 protein; (d) the antibody or antigen-binding fragment thereof inhibits the binding of KLRAP1 to the HCMV UL144 protein; or (e) the antibody or antigen-binding fragment thereof inhibits the binding of SGCA to the HCMV RL10 protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has a congenital HCMV infection. In some aspects, the individual has CMV-related allograft rejection. In some aspects, the individual is a human.

### D. Methods of Treating Individuals Having VZV Infections

In some aspects, the disclosure features a method of treating an individual having a Varicella zoster virus (VZV) infection or preventing a VZV infection in an individual comprising administering to the individual an effective amount of an ICAM1 antagonist, a MUSK antagonist, a HAVCR1 antagonist, a MOG antagonist, or a KIAA0319L antagonist.

In some aspects, the disclosure features a method of decreasing VZV infection in an individual comprising administering to the individual an effective amount of an ICAM1 antagonist, a MUSK antagonist, a HAVCR1 antagonist, a MOG antagonist, or a KIAA0319L antagonist.

In some aspects, the disclosure features a method of reducing VZV attachment to a cell of an individual comprising administering to the individual an effective amount of a an ICAM1 antagonist, a MUSK antagonist, a HAVCR1 antagonist, a MOG antagonist, or a KIAA0319L antagonist. In some aspects, the administering comprises contacting the cell of the individual with an effective amount of an ICAM1 antagonist, a MUSK antagonist, a HAVCR1 antagonist, a MOG antagonist, or a KIAA0319L antagonist.

In some aspects, (a) the ICAM1 antagonist results in a decrease in the binding of ICAM1 and the VZV glycoprotein C (gC) protein relative to binding of the two proteins in the absence of the antagonist; (b) the MUSK antagonist results in a decrease in the binding of MUSK and the VZV glycoprotein B (gB) protein relative to binding of the two proteins in the absence of the antagonist; (c) the HAVCR1 antagonist results in a decrease in the binding of HAVCR1 and the VZV gB protein relative to binding of the two proteins in the absence of the antagonist; (d) the MOG antagonist results in a decrease in the binding of MOG and the VZV glycoprotein I (gI) protein relative to binding of the two proteins in the absence of the antagonist; or (e) the KIAA0319L antagonist results in a decrease in the binding of KIAA0319L and the VZV gI protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the ICAM1 antagonist, MUSK antagonist, HAVCR1 antagonist, MOG antagonist, or KIAA0319L antagonist reduces the extent and/or severity of VZV infection of the individual relative to infection in the absence of the ICAM1 antagonist, MUSK antagonist, HAVCR1 antagonist, MOG antagonist, or KIAA0319L antagonist, respectively.

In some aspects, the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the ICAM1 antagonist, MUSK antagonist, HAVCR1 antagonist, MOG antagonist, or KIAA0319L antagonist is a peptide.

In some aspects, the ICAM1 antagonist, MUSK antagonist, HAVCR1 antagonist, MOG antagonist, or KIAA0319L antagonist is an antibody or antigen-binding fragment thereof. In some aspects, (a) the antibody or antigen-binding fragment thereof binds the VZV gC protein and inhibits its binding to ICAM1; (b) the antibody or antigen-binding fragment thereof binds the VZV gB protein and inhibits its binding to MUSK and/or HAVCR1; or (c) the antibody or antigen-binding fragment thereof binds the VZV gI protein and inhibits its binding to MOG and/or KIAA0319L. In some aspects, the antibody or antigen-binding fragment thereof binds ICAM1, MUSK, HAVCR1, MOG, or KIAA0319L. In some aspects, (a) the antibody or antigen-binding fragment thereof inhibits the binding of ICAM1 to the VZV gC protein; (b) the antibody or antigen-binding fragment thereof inhibits the binding of MUSK or HAVCR1 to the VZV gB protein; or (c) the antibody or antigen-binding fragment thereof inhibits the binding of MOG or KIAA0319L to the VZV gI protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has chicken pox or shingles. In some aspects, the individual is a human.

### E. Methods of Treating Individuals Having HHV8 Infections

In some aspects, the disclosure features a method of treating an individual having a human herpesvirus 8 (HHV8) infection or preventing a HHV8 infection in an individual comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

In some aspects, the disclosure features a method of decreasing HHV8 infection in an individual comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

In some aspects, the disclosure features a method of reducing HHV8 attachment to a cell of an individual comprising administering to the individual an effective amount of a KLRAP1 antagonist, a LILRB1 antagonist, a CLEC4G antagonist, a FLRT1 antagonist, a FLRT2 antagonist, or a FLRT3 antagonist. In some aspects, the administering comprises contacting the cell of the individual with an effective amount of a KLRAP1 antagonist, a LILRB1 antagonist, a CLEC4G antagonist, a FLRT1 antagonist, a FLRT2 antagonist, or a FLRT3 antagonist.

In some aspects, (a) the KLRAP1 antagonist results in a decrease in the binding of KLRAP1 and the HHV8 K14 protein relative to binding of the two proteins in the absence of the antagonist; (b) the LILRB1 antagonist results in a decrease in the binding of LILRB1 and the HHV8 KCP protein relative to binding of the two proteins in the absence of the antagonist; (c) the CLEC4G antagonist results in a decrease in the binding of CLEC4G and the HHV8 KCP protein relative to binding of the two proteins in the absence of the antagonist; (d) the FLRT1 antagonist results in a decrease in the binding of FLRT1 and the HHV8 KCP protein relative to binding of the two proteins in the absence of the antagonist; (e) the FLRT2 antagonist results in a decrease in the binding of FLRT2 and the HHV8 KCP protein relative to binding of the two proteins in the absence of the antagonist; or (f) the FLRT3 antagonist results in a decrease in the binding of FLRT3 and the HHV8 KCP protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the KLRAP1 antagonist, LILRB1 antagonist, CLEC4G antagonist, FLRT1 antagonist, FLRT2 antagonist, or FLRT3 antagonist reduces the extent and/or severity of HHV8 infection of the individual relative to infection in the absence of the KLRAP1 antagonist, LILRB1 antagonist, CLEC4G antagonist, FLRT1 antagonist, FLRT2 antagonist, or FLRT3 antagonist, respectively.

In some aspects, the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the KLRAP1 antagonist, LILRB1 antagonist, CLEC4G antagonist, FLRT1 antagonist, FLRT2 antagonist, or FLRT3 antagonist is a peptide.

In some aspects, the KLRAP1 antagonist, LILRB1 antagonist, CLEC4G antagonist, FLRT1 antagonist, FLRT2 antagonist, or FLRT3 antagonist is an antibody or antigen-binding fragment thereof. In some aspects, (a) the antibody or antigen-binding fragment thereof binds the HHV8 K14 protein and inhibits its binding to KLRAP1; or (b) the antibody or antigen-binding fragment thereof binds the HHV8 KCP protein and inhibits its binding to LILRB1, CLEC4G, FLRT1, FLRT2, and/or FLRT3. In some aspects, the antibody or antigen-binding fragment thereof binds KLRAP1, LILRB1, CLEC4G, FLRT1, FLRT2, or FLRT3. In some aspects, (a) the antibody or antigen-binding fragment thereof inhibits the binding of KLRAP1 to the HHV8 K14 protein; or (b) the antibody or antigen-binding fragment thereof inhibits the binding of LILRB1, CLEC4G, FLRT1, FLRT2, or FLRT3 to the HHV8 KCP protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has Kaposi's sarcoma, primary effusion lymphoma, HHV8-associated multicentric Castleman's disease, or KSHV inflammatory cytokine syndrome. In some aspects, the individual is a human.

### F. Methods of Prophylaxis Against Herpesvirus Infections

In some aspects, the disclosure features a methods prophylaxis against infection of an individual by a herpesvirus, e.g., methods of preventing a HSV-2 infection in an individual comprising administering to the individual an effective amount of a CSPG5 antagonist, a PRRG2 antagonist, a UNC5D antagonist, or a PLB1 antagonist; preventing a MCHV infection in an individual comprising administering to the individual an effective amount of a PILRA antagonist; preventing a HCMV infection in an individual comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, a or a SGCA antagonist; preventing a VZV infection in an individual comprising administering to the individual an effective amount of an ICAM1 antagonist, a MUSK antagonist, a HAVCR1 antagonist, a MOG antagonist, or a KIAA0319L antagonist; preventing a HHV8 infection in an individual comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

In some aspects, infection of an individual by a herpesvirus is decreased or eliminated in patients treated according to the above-described methods relative to untreated patients or relative to patients treated using a control method (e.g., SOC), e.g., decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% (e.g., decreased by 5%-15%, 15%-25%, 25%-35%, 35%-45%, 45%-55%, 55%-65%, 65%-75%, 75%-85%, 85%-95%, or 95%-100%).

In some aspects, the treatment reduces the extent and/or severity of herpesvirus infection in the individual relative to infection in the absence of the treatment. In some aspects, the extent and/or severity of herpesvirus infection is decreased in patients treated according to the above-described methods relative to untreated patients or relative to patients treated using a control method (e.g., SOC), e.g., decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% (e.g., decreased by 5%-15%, 15%-25%, 25%-35%, 35%-45%, 45%-55%, 55%-65%, 65%-75%, 75%-85%, 85%-95%, or 95%-100%).

### G. Combination Therapies

In some aspects of the methods, medicaments, and uses for treatment and prophylaxis described herein, the method comprises administering to the individual at least one additional therapy (e.g., one, two, three, four, or more than four additional therapies). The CSPG5 antagonist, PRRG2 antagonist, UNC5D antagonist, PLB1 antagonist, PILRA antagonist, VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, SGCA antagonist, ICAM1 antagonist, MUSK antagonist, HAVCR1 antagonist, MOG antagonist, KIAA0319L antagonist, KLRAP1 antagonist, LILRB1 antagonist, CLEC4G antagonist, FLRT1 antagonist, FLRT2 antagonist, or FLRT3 antagonist may be administered to the individual prior to, concurrently with, or after the at least one additional therapy (e.g., a therapy comprising an agent for treating or preventing herpesvirus infection).

### H. Methods of Delivery

The compositions utilized in the methods, medicaments, and uses described herein (e.g., a modulator of an interaction between a herpesvirus surface protein and a host cell surface protein, e.g., a small molecule, an antibody, an antigen-binding fragment, a peptide, a mimic, an antisense oligonucleotide, or an siRNA) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, a modulator of a protein-protein interaction is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A modulator of a protein-protein interaction described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modulator need not be, but is optionally formulated with and/or administered concurrently with one or more agents currently used to prevent or treat the disorder in question. The effective amount of such other agents depends on the amount of the modulator present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

All patent, patent publication and literature references cited in the present specification are hereby incorporated by reference in their entirety.

### V. MEDICAMENTS AND USES

### A. Medicaments for Treatment of HSV-2 Infections

In some aspects, the disclosure features a CSPG5 antagonist, a PRRG2 antagonist, a UNC5D antagonist, or a PLB1 antagonist for use as a medicament. In some aspects, the medicament is for treating an HSV-2 infection. In some aspects, the medicament is for treating genital herpes or herpes simplex encephalitis.

In some aspects, the disclosure features use of a CSPG5 antagonist, a PRRG2 antagonist, a UNC5D antagonist, or a PLB1 antagonist in the manufacture of a medicament for treatment of an HSV-2 infection.

In some aspects, the disclosure features use of a CSPG5 antagonist, a PRRG2 antagonist, a UNC5D antagonist, or a PLB1 antagonist in the manufacture of a medicament for treatment of genital herpes or herpes simplex encephalitis.

In some aspects, the disclosure features use of a CSPG5 antagonist, a PRRG2 antagonist, a UNC5D antagonist, or a PLB1 antagonist in the manufacture of a medicament for reducing or preventing infection of a cell by HSV-2.

In some aspects, (a) the CSPG5 antagonist results in a decrease in the binding of CSPG5 and the HSV-2 glycoprotein G (gG) protein relative to binding of the two proteins in the absence of the antagonist; (b) the PRRG2 antagonist results in a decrease in the binding of PRRG2 and the HSV-2 gG protein relative to binding of the two proteins in the absence of the antagonist; (c) the UNC5D antagonist results in a decrease in the binding of UNC5D and the HSV-2 gG protein relative to binding of the two proteins in the absence of the antagonist; or (d) the PLB1 antagonist results in a decrease in the binding of PLB1 and the HSV-2 gD protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the CSPG5 antagonist, PRRG2 antagonist, UNC5D antagonist, or PLB1 antagonist is a peptide.

In some aspects, the CSPG5 antagonist, PRRG2 antagonist, UNC5D antagonist, or PLB1 antagonist is an antibody or antigen-binding fragment thereof. In some aspects, (a) the antibody or antigen-binding fragment thereof binds the HSV-2 gG protein and inhibits its binding to CSPG5, PRRG2, and/or UNC5D; or (b) the antibody or antigen-binding fragment thereof binds the HSV-2 gD protein and inhibits its binding to PLB1. In some aspects, the antibody or antigen-binding fragment thereof binds CSPG5, PRRG2, UNC5D, or PLB1. In some aspects, (a) the antibody or antigen-binding fragment thereof inhibits the binding of CSPG5, PRRG2, or UNC5D to the HSV-2 gG protein; or (b) the antibody or antigen-binding fragment thereof inhibits the binding of PLB1 to the HSV-2 gD protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has genital herpes or herpes simplex encephalitis. In some aspects, the individual is a human.

### B. Medicaments for Treatment of MCHV Infections

In some aspects, the disclosure features a PILRA antagonist for use as a medicament for treating an MCHV infection, e.g., a zoonotic MCHV infection.

In some aspects, the disclosure features use of a PILRA antagonist in the manufacture of a medicament for treatment of a MCHV infection, e.g., a zoonotic MCHV infection.

In some aspects, the disclosure features use of a PILRA antagonist in the manufacture of a medicament for reducing or preventing infection of a cell by MCHV.

In some aspects, the PILRA antagonist results in a decrease in the binding of PILRA and the MCHV glycoprotein G (gG) protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the PILRA antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the PILRA antagonist is a peptide.

In some aspects, the PILRA antagonist is an antibody or antigen-binding fragment thereof. In some aspects, the antibody or antigen-binding fragment thereof binds the MCHV gG protein and inhibits its binding to PILRA. In some aspects, the antibody or antigen-binding fragment thereof binds the MCHV gG protein and inhibits its binding to PILRA. In some aspects, the antibody or antigen-binding fragment thereof inhibits the binding of PILRA to the MCHV gG protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

In some aspects, the antibody is a bispecific antibody.

In some aspects, the individual has a zoonotic MCHV infection, e.g., an MCHV infection transmitted by a macaque monkey to a human. In some aspects, the individual is a human.

### C. Medicaments for Treatment of HCMV Infections

In some aspects, the disclosure features a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist for use as a medicament. In some aspects, the medicament is for treating a HCMV infection. In some aspects, the medicament is for treating CMV-related allograft rejection.

In some aspects, the disclosure features use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for treatment of an HCMV infection.

In some aspects, the disclosure features use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for treatment of CMV-related allograft rejection.

In some aspects, the disclosure features use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for reducing or preventing infection of a cell by HCMV.

In some aspects, (a) the VEGFR2 antagonist results in a decrease in the binding of VEGFR2 and the HCMV UL6 protein relative to binding of the two proteins in the absence of the antagonist; (b) the MERTK antagonist results in a decrease in the binding of MERTK and the HCMV UL6 protein relative to binding of the two proteins in the absence of the antagonist; (c) the PDGFRa antagonist results in a decrease in the binding of PDGFRa and the HCMV UL6 protein relative to binding of the two proteins in the absence of the antagonist; (d) the KIRREL2 antagonist results in a decrease in the binding of KIRREL2 and the HCMV UL6 protein relative to binding of the two proteins in the absence of the antagonist; (e) the LILRB5 antagonist results in a decrease in the binding of LILRB5 and the HCMV UL9 protein relative to binding of the two proteins in the absence of the antagonist; (f) the ULBP1 antagonist results in a decrease in the binding of ULBP1 and the HCMV UL9 protein relative to binding of the two proteins in the absence of the antagonist; (g) the KIR2DL3 antagonist results in a decrease in the binding of KIR2DL3 and the HCMV UL9 protein relative to binding of the two proteins in the absence of the antagonist; (h) the KIR2DS1 antagonist results in a decrease in the binding of KIR2DS1 and the HCMV UL9 protein relative to binding of the two proteins in the absence of the antagonist; (i) the KIR2DS2 antagonist results in a decrease in the binding of KIR2DS2 and the HCMV UL9 protein relative to binding of the two proteins in the absence of the antagonist; (j) the KIR2DS4 antagonist results in a decrease in the binding of KIR2DS4 and the HCMV UL9 protein relative to binding of the two proteins in the absence of the antagonist; (k) the KIR2DS5 antagonist results in a decrease in the binding of KIR2DS5 and the HCMV UL9 protein relative to binding of the two proteins in the absence of the antagonist; (l) the KIR2DL1 antagonist results in a decrease in the binding of KIR2DL1 and the HCMV UL9 protein relative to binding of the two proteins in the absence of the antagonist; (m) the KIR3DL1 antagonist results in a decrease in the binding of KIR3DL1 and the HCMV UL9 protein relative to binding of the two proteins in the absence of the antagonist; (n) the PRRG2 antagonist results in a decrease in the binding of PRRG2 and the HCMV UL142 protein relative to binding of the two proteins in the absence of the antagonist; (o) the KLRAP1 antagonist results in a decrease in the binding of KLRAP1 and the HCMV UL144 protein relative to binding of the two proteins in the absence of the antagonist; or (p) the SGCA antagonist results in a decrease in the binding of SGCA and the HCMV RL10 protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

In some aspects, the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof. In some aspects, (a) the antibody or antigen-binding fragment thereof binds the HCMV UL6 protein and inhibits its binding to VEGFR2, MERTK, PDGFRa, and/or KIRREL2; (b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1; (c) the antibody or antigen-binding fragment thereof binds the HCMV UL142 protein and inhibits its binding to PRRG2; (d) the antibody or antigen-binding fragment thereof binds the HCMV UL144 protein and inhibits its binding to KLRAP1; or (e) the antibody or antigen-binding fragment thereof binds the HCMV RL10 protein and inhibits its binding to SGCA. In some aspects, the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA. In some aspects, (a) the antibody or antigen-binding fragment thereof inhibits the binding of VEGFR2, MERTK, PDGFRa, or KIRREL2 to the HCMV UL6 protein; (b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein; (c) the antibody or antigen-binding fragment thereof inhibits the binding of PRRG2 to the HCMV UL142 protein; (d) the antibody or antigen-binding fragment thereof inhibits the binding of KLRAP1 to the HCMV UL144 protein; or (e) the antibody or antigen-binding fragment thereof inhibits the binding of SGCA to the HCMV RL10 protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

### D. Medicaments for Treatment of VZV Infections

In some aspects, the disclosure features an ICAM1 antagonist, a MUSK antagonist, a HAVCR1 antagonist, a MOG antagonist, or a KIAA0319L antagonist for use as a medicament. In some aspects, the medicament is for treating a VZV infection. In some aspects, the medicament is for treating chicken pox or shingles.

In some aspects, the disclosure features use of an ICAM1 antagonist, a MUSK antagonist, a HAVCR1 antagonist, a MOG antagonist, or a KIAA0319L antagonist in the manufacture of a medicament for treatment of a VZV infection.

In some aspects, the disclosure features use of an ICAM1 antagonist, a MUSK antagonist, a HAVCR1 antagonist, a MOG antagonist, or a KIAA0319L antagonist in the manufacture of a medicament for treatment of chicken pox or shingles.

In some aspects, the disclosure features use of an ICAM1 antagonist, a MUSK antagonist, a HAVCR1 antagonist, a MOG antagonist, or a KIAA0319L antagonist in the manufacture of a medicament for reducing or preventing infection of a cell by VZV.

In some aspects, (a) the ICAM1 antagonist results in a decrease in the binding of ICAM1 and the VZV glycoprotein C (gC) protein relative to binding of the two proteins in the absence of the antagonist; (b) the MUSK antagonist results in a decrease in the binding of MUSK and the VZV glycoprotein B (gB) protein relative to binding of the two proteins in the absence of the antagonist; (c) the HAVCR1 antagonist results in a decrease in the binding of HAVCR1 and the VZV gB protein relative to binding of the two proteins in the absence of the antagonist; (d) the MOG antagonist results in a decrease in the binding of MOG and the VZV glycoprotein I (gI) protein relative to binding of the two proteins in the absence of the antagonist; or (e) the KIAA0319L antagonist results in a decrease in the binding of KIAA0319L and the VZV gI protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the ICAM1 antagonist, MUSK antagonist, HAVCR1 antagonist, MOG antagonist, or KIAA0319L antagonist is a peptide.

In some aspects, the ICAM1 antagonist, MUSK antagonist, HAVCR1 antagonist, MOG antagonist, or KIAA0319L antagonist is an antibody or antigen-binding fragment thereof. In some aspects, (a) the antibody or antigen-binding fragment thereof binds the VZV gC protein and inhibits its binding to ICAM1; (b) the antibody or antigen-binding fragment thereof binds the VZV gB protein and inhibits its binding to MUSK and/or HAVCR1; or (c) the antibody or antigen-binding fragment thereof binds the VZV gI protein and inhibits its binding to MOG and/or KIAA0319L. In some aspects, the antibody or antigen-binding fragment thereof binds ICAM1, MUSK, HAVCR1, MOG, or KIAA0319L. In some aspects, (a) the antibody or antigen-binding fragment thereof inhibits the binding of ICAM1 to the VZV gC protein; (b) the antibody or antigen-binding fragment thereof inhibits the binding of MUSK or HAVCR1 to the VZV gB protein; or (c) the antibody or antigen-binding fragment thereof inhibits the binding of MOG or KIAA0319L to the VZV gI protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

### E. Medicaments for Treatment of HHV8 Infections

In some aspects, the disclosure features a KLRAP1 antagonist, a LILRB1 antagonist, a CLEC4G antagonist, a FLRT1 antagonist, a FLRT2 antagonist, or a FLRT3 antagonist for use as a medicament. In some aspects, the medicament is for treating an HHV8 infection. In some aspects, the medicament is for treating Kaposi's sarcoma, primary effusion lymphoma, HHV8-associated multicentric Castleman's disease, or KSHV inflammatory cytokine syndrome.

In some aspects, (a) the KLRAP1 antagonist results in a decrease in the binding of KLRAP1 and the HHV8 K14 protein relative to binding of the two proteins in the absence of the antagonist; (b) the LILRB1 antagonist results in a decrease in the binding of LILRB1 and the HHV8 KCP protein relative to binding of the two proteins in the absence of the antagonist; (c) the CLEC4G antagonist results in a decrease in the binding of CLEC4G and the HHV8 KCP protein relative to binding of the two proteins in the absence of the antagonist; (d) the FLRT1 antagonist results in a decrease in the binding of FLRT1 and the HHV8 KCP protein relative to binding of the two proteins in the absence of the antagonist; (e) the FLRT2 antagonist results in a decrease in the binding of FLRT2 and the HHV8 KCP protein relative to binding of the two proteins in the absence of the antagonist; or (f) the FLRT3 antagonist results in a decrease in the binding of FLRT3 and the HHV8 KCP protein relative to binding of the two proteins in the absence of the antagonist. In some aspects, the decrease in binding is at least 5%, at least 10%, at least 20%, at least 30%, at least 40%, at least 50%, at least 60%, at least 70%, at least 80%, at least 90%, or 100% (e.g., 5%-15%, 15%-25%, 25%-35%, 35%-45%, 45%-55%, 55%-65%, 65%-75%, 75%-85%, 85%-95%, or 95%-100%). In some aspects, the decrease in binding is at least 40%.

In some aspects, the disclosure features use of a KLRAP1 antagonist, a LILRB1 antagonist, a CLEC4G antagonist, a FLRT1 antagonist, a FLRT2 antagonist, or a FLRT3 antagonist in the manufacture of a medicament for treatment of an HHV8 infection.

In some aspects, the disclosure features use of a KLRAP1 antagonist, a LILRB1 antagonist, a CLEC4G antagonist, a FLRT1 antagonist, a FLRT2 antagonist, or a FLRT3 antagonist in the manufacture of a medicament for treatment of Kaposi's sarcoma, primary effusion lymphoma, HHV8-associated multicentric Castleman's disease, or KSHV inflammatory cytokine syndrome.

In some aspects, the disclosure features use of a KLRAP1 antagonist, a LILRB1 antagonist, a CLEC4G antagonist, a FLRT1 antagonist, a FLRT2 antagonist, or a FLRT3 antagonist in the manufacture of a medicament for reducing or preventing infection of a cell by HHV8.

In some aspects, the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

In some aspects, the inhibitory nucleic acid is an antisense oligonucleotide (ASO) or a small interfering RNA (siRNA).

In some aspects, the KLRAP1 antagonist, LILRB1 antagonist, CLEC4G antagonist, FLRT1 antagonist, FLRT2 antagonist, or FLRT3 antagonist is a peptide.

In some aspects, the KLRAP1 antagonist, LILRB1 antagonist, CLEC4G antagonist, FLRT1 antagonist, FLRT2 antagonist, or FLRT3 antagonist is an antibody or antigen-binding fragment thereof. In some aspects, (a) the antibody or antigen-binding fragment thereof binds the HHV8 K14 protein and inhibits its binding to KLRAP1; or (b) the antibody or antigen-binding fragment thereof binds the HHV8 KCP protein and inhibits its binding to LILRB1, CLEC4G, FLRT1, FLRT2, and/or FLRT3. In some aspects, the antibody or antigen-binding fragment thereof binds KLRAP1, LILRB1, CLEC4G, FLRT1, FLRT2, or FLRT3. In some aspects, (a) the antibody or antigen-binding fragment thereof inhibits the binding of KLRAP1 to the HHV8 K14 protein; or (b) the antibody or antigen-binding fragment thereof inhibits the binding of LILRB1, CLEC4G, FLRT1, FLRT2, or FLRT3 to the HHV8 KCP protein.

In some aspects, the antigen-binding fragment is a bis-Fab, an Fv, a Fab, a Fab′-SH, a F(ab′)₂, a diabody, a linear antibody, an scFv, an scFab, a VH domain, or a VHH domain.

### VI. EXAMPLES

### Example 1. AVEXIS Screening for Herpesvirus Surface Protein Interactions

Avidity-based extracellular interaction screening (AVEXIS) screening (see, e.g., Bushell et al., Genome Res, 18: 622-630, 2008; Martinez-Martin et al., J Immunol Res, 2197615, 2017; and PCT/US2020/025471, each of which is incorporated by reference herein in its entirety) was performed for the herpesvirus proteins of Table 1. Briefly, extracellular domains (ECDs) of the proteins of Table 1 were pentamerized using the pentamerization domain of rat cartilaginous oligomeric matrix protein (COMP) and were screened for interaction with a library of human single-pass transmembrane (STM) receptors. Identified interactions are reported in Table 3.

### A. HSV-2 gG and gD Proteins

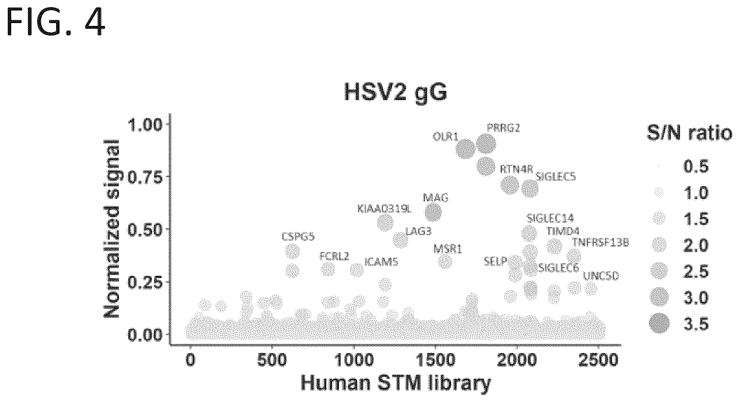

Results of the screen for interaction between the herpes simplex virus 2 (HSV-2) glycoprotein G (gG) and the STM library are shown in FIG. 4

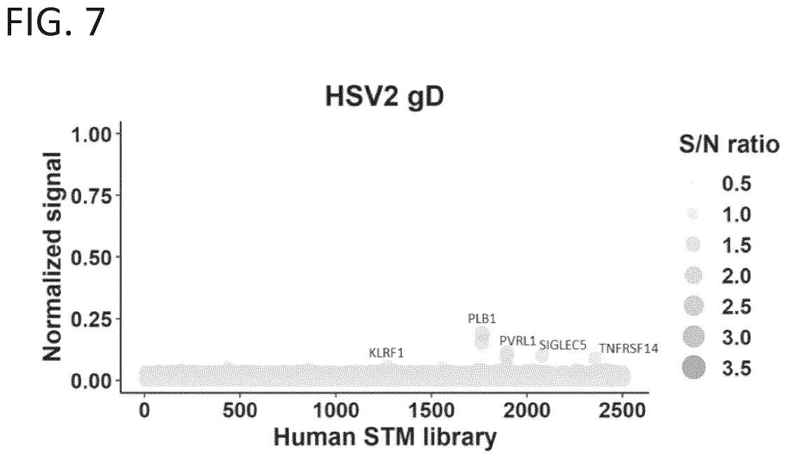

Results of the screen for interaction between HSV-2 glycoprotein D (gD) and the STM library are shown in FIG. 7

### B. MCHV Proteins

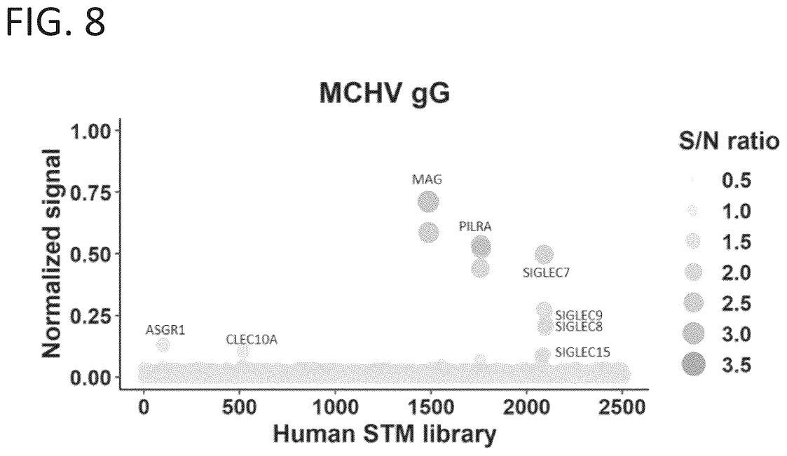

Results of the screen for interaction between macacine alphaherpesvirus (MCHV) glycoprotein G (gG) and the STM library are shown in FIG. 8

### C. HCMV Proteins

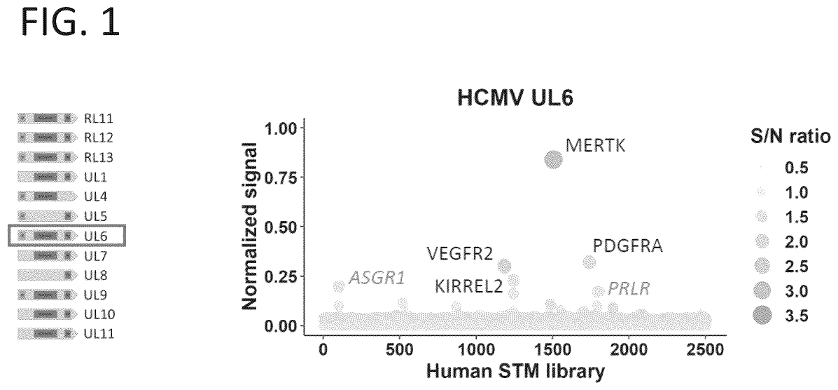

Results of the screen for interaction between the human cytomegalovirus (HCMV) protein UL6 and the STM library are shown in FIG. 1

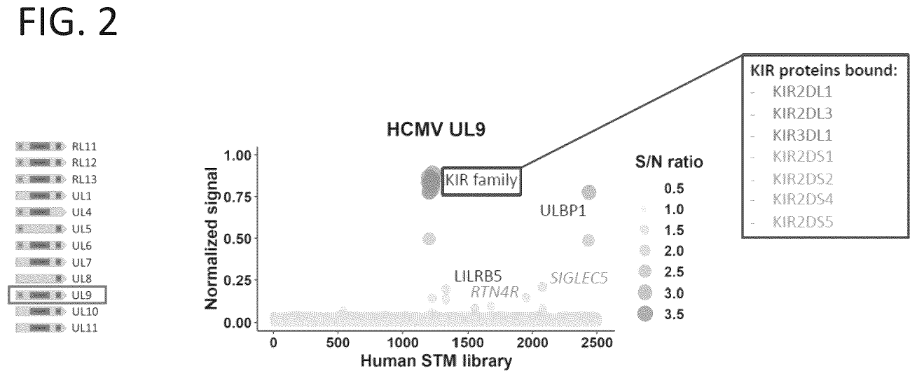

Results of the screen for interaction between the human cytomegalovirus (HCMV) protein UL9 and the STM library are shown in FIG. 2

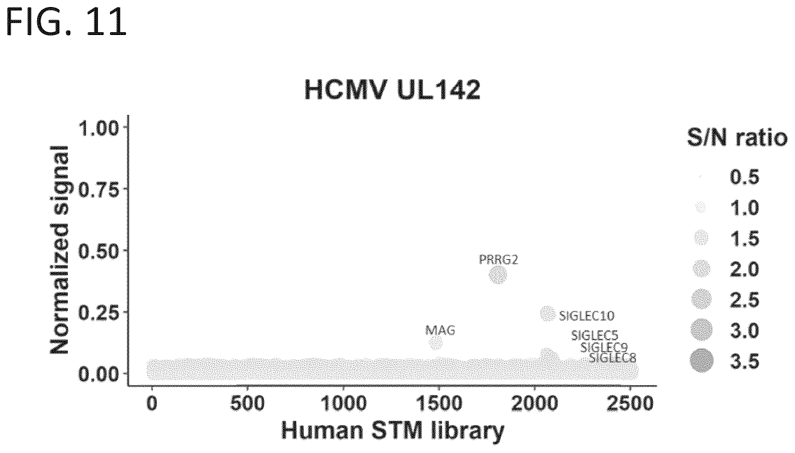

Results of the screen for interaction between the human cytomegalovirus (HCMV) protein UL142 and the STM library are shown in FIG. 11

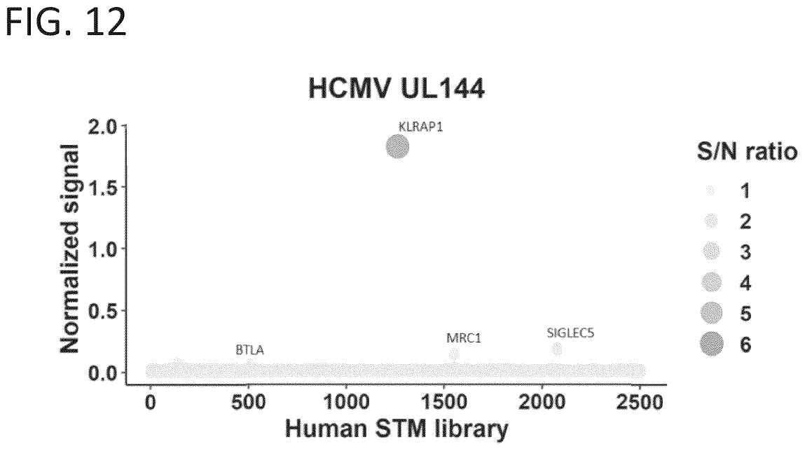

Results of the screen for interaction between the human cytomegalovirus (HCMV) protein UL144 and the STM library are shown in FIG. 12.

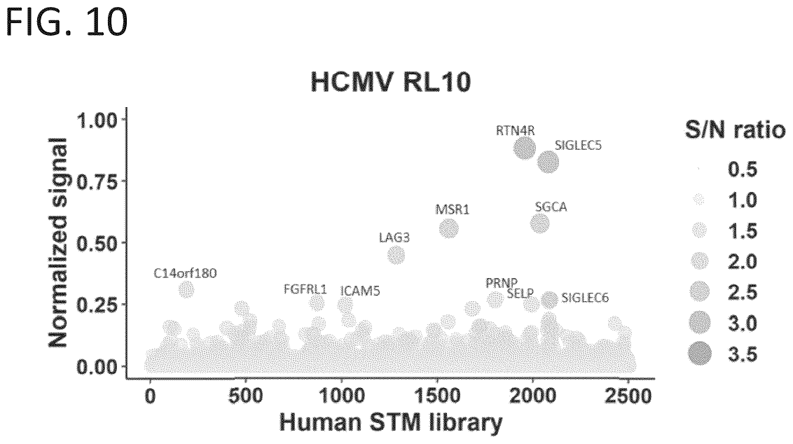

Results of the screen for interaction between the human cytomegalovirus (HCMV) protein RL10 and the STM library are shown in FIG. 10.

### D. VZV Proteins

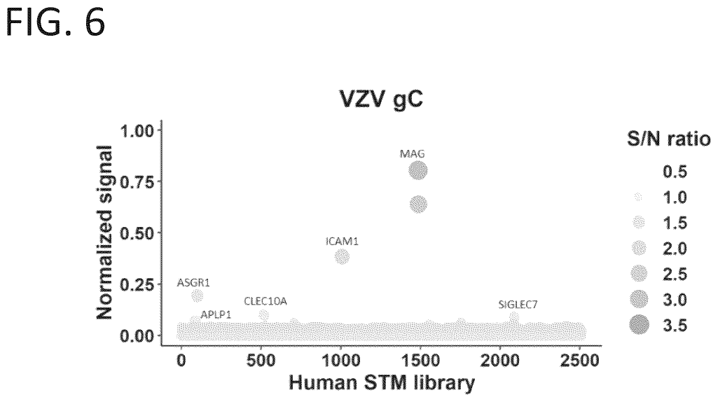

Results of the screen for interaction between the Varicella zoster virus (VZV) glycoprotein C (gC) and the STM library are shown in FIG. 6

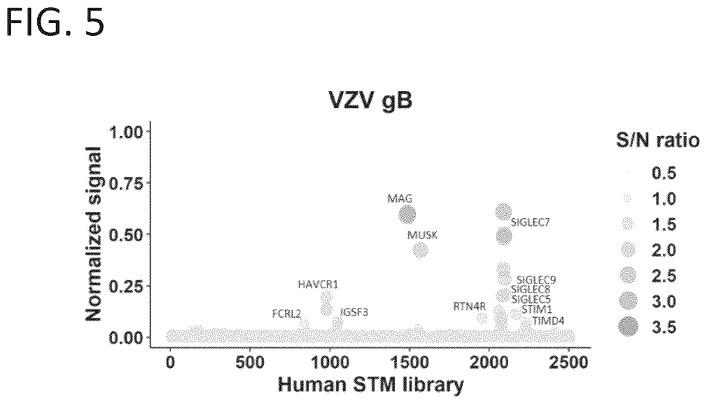

Results of the screen for interaction between the VZV glycoprotein B (gB) and the STM library are shown in FIG. 5.

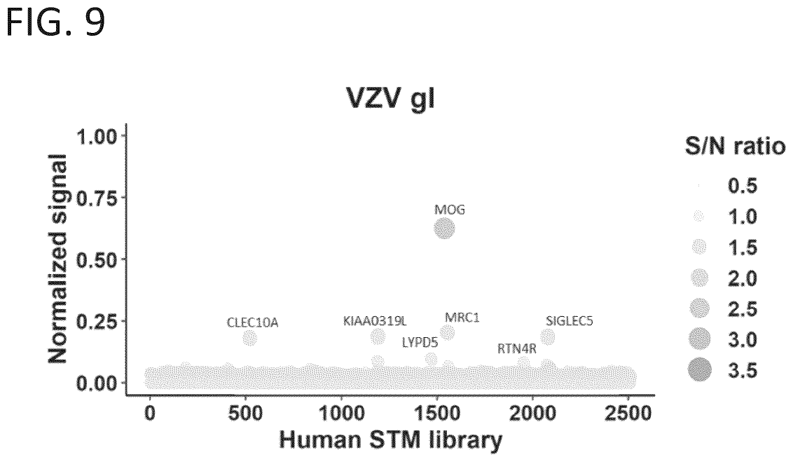

Results of the screen for interaction between the VZV glycoprotein I (gI) and the STM library are shown in FIG. 9.

### E. HHV8

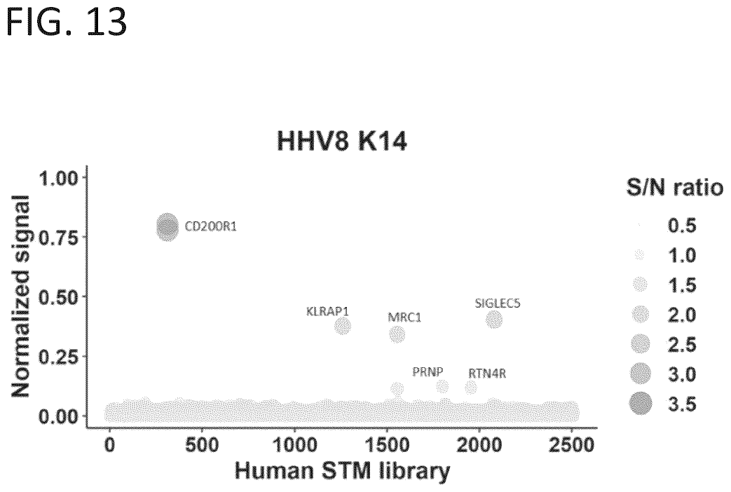

Results of the screen for interaction between the human herpesvirus 8 (HHV8) K14 protein and the STM library are shown in FIG. 13

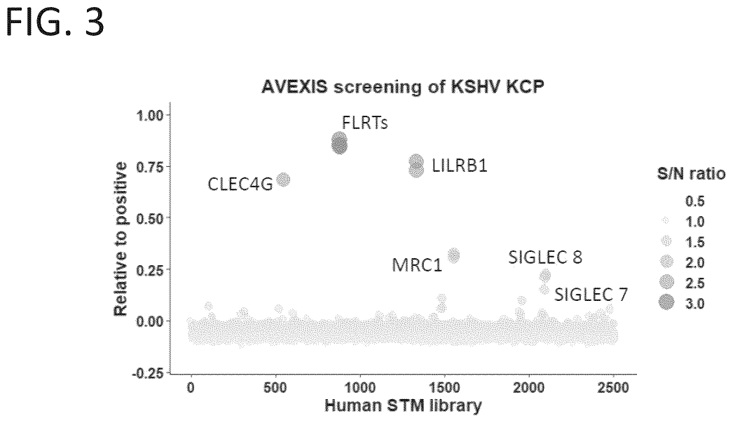

Results of the screen for interaction between the HHV8 KCP protein and the STM library are shown in FIG. 3.

### Example 2. Validation of Herpesvirus Surface Protein Interactions

Interactions identified in Example 1 were validated using cell-based immunofluorescence assays.

### A. HSV-2 gG and gD Proteins

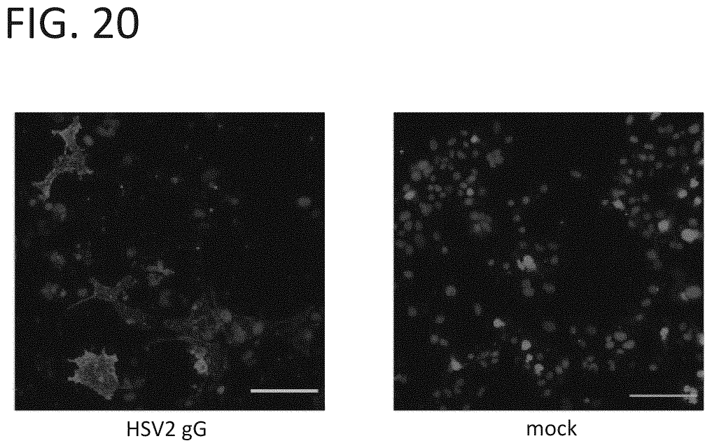

Results of the validation assays for HSV-2 gG interactions are shown in FIG. 20. CSPG5, PRRG2, and UNC5D were identified as interactors.

### B. HCMV Proteins

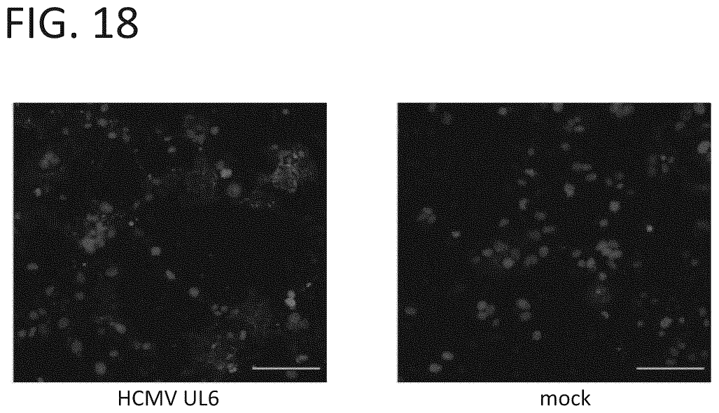

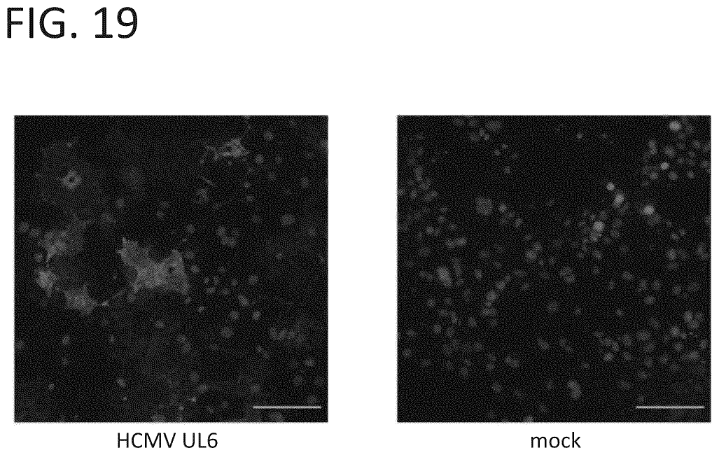

Results of the validation assays for HCMV UL6 interactions are shown in FIGS. 18 and 19. VEGFR2, MERTK, PDGFRa, and KIRREL2 were identified as interactors.

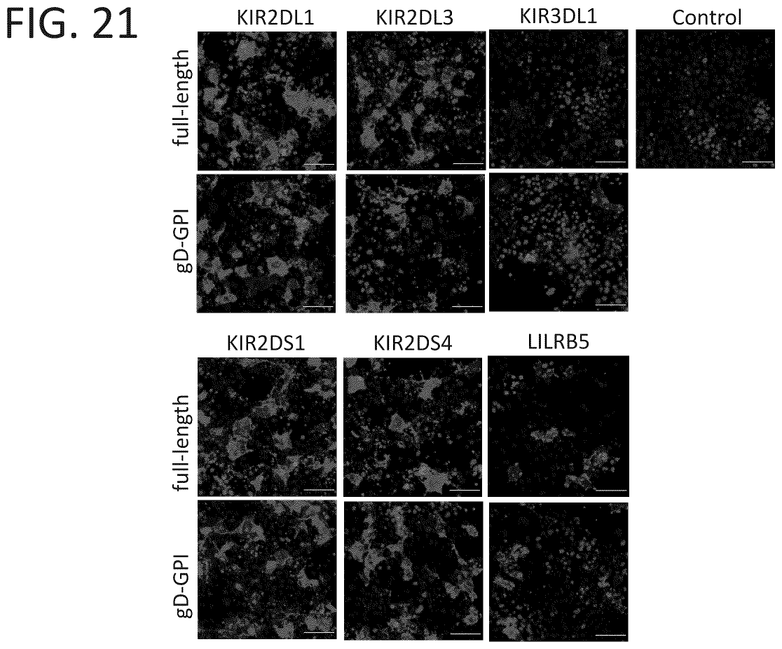

Results of the validation assays for HCMV UL9 interactions are shown in FIG. 21. LILRB5, KIR2DL3, KIR2DS1, KIR2DS4, KIR2DL1, and KIR3DL1 were identified as interactors.

### C. VZV Proteins

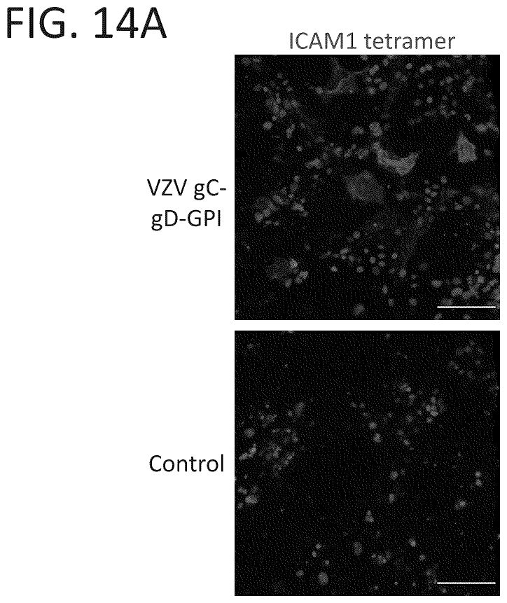

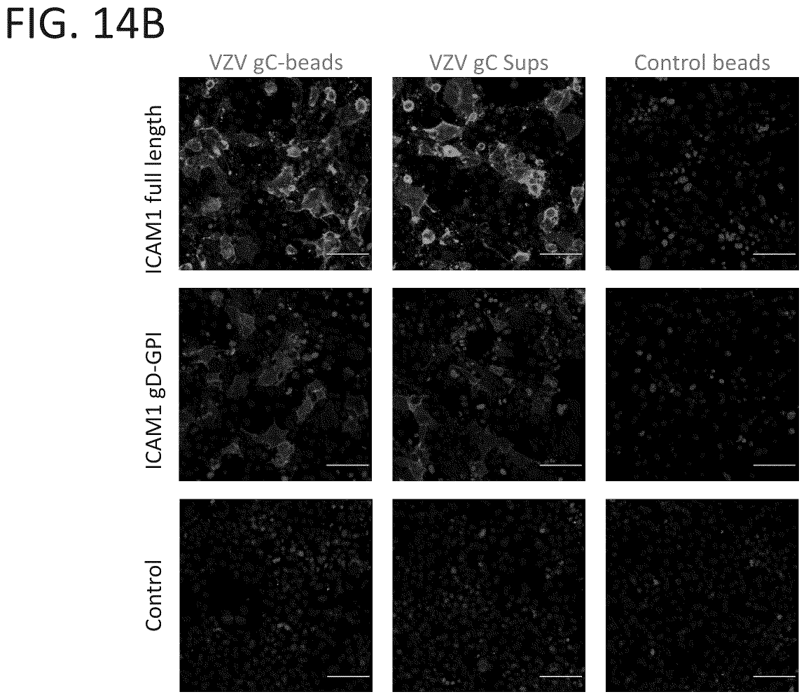

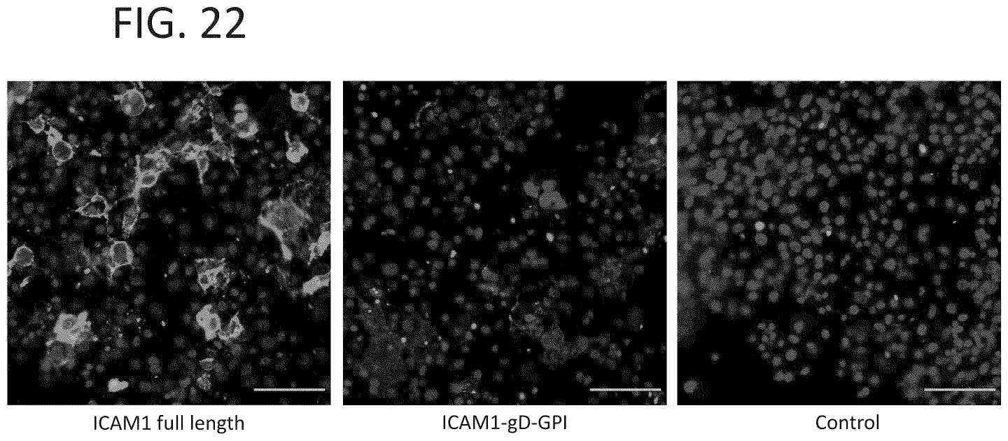

Results of the validation assays for VZV gC interactions are shown in FIGS. 14A, 14B, and 22. ICAM1 was identified as an interactor.

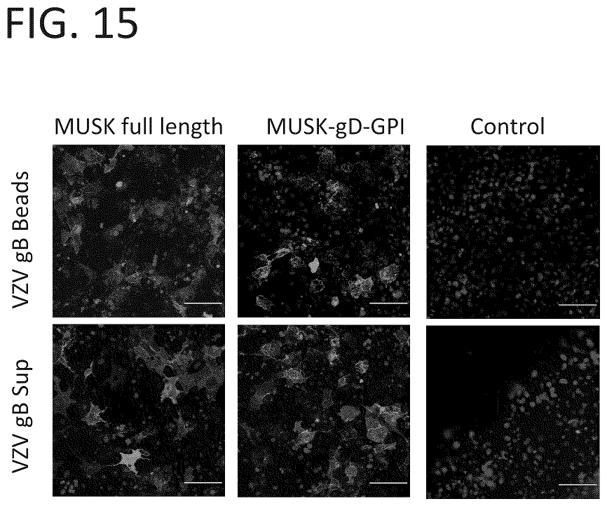

Results of the validation assays for VZV gB interactions are shown in FIGS. 15 and 16. MUSK and HAVCR1 were identified as interactors.

### D. HHV8

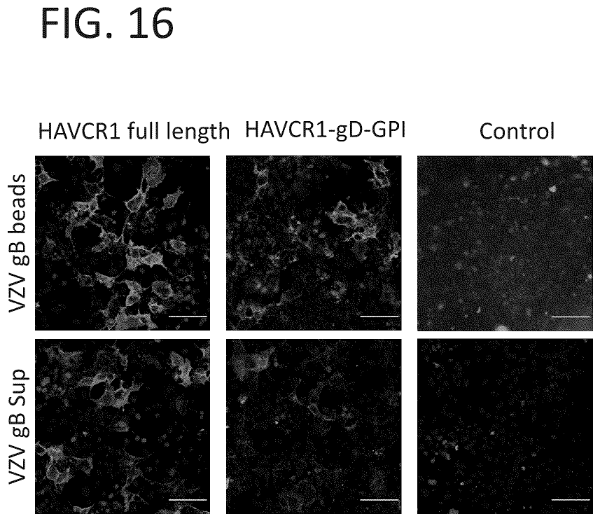

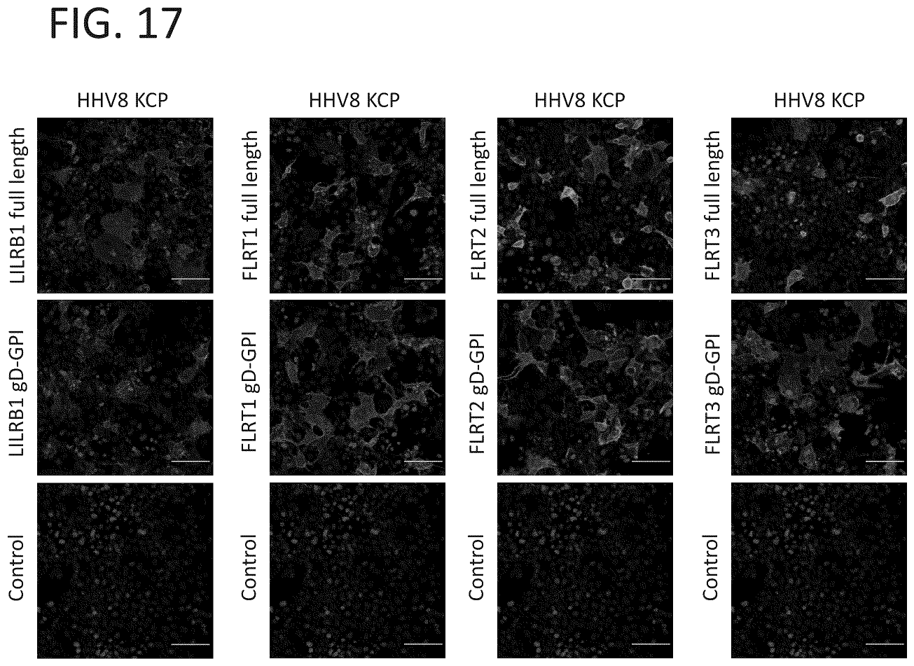

Results of the validation assays for HHV8 KCP interactions are shown in FIG. 17. LILRB1, FLRT1, FLRT2, and FLRT3 were identified as interactors.

### Example 3. Interactions between hCMV UL6 and the Human Receptors VEGFR2 and MERTK

### A. Validation of Interactions

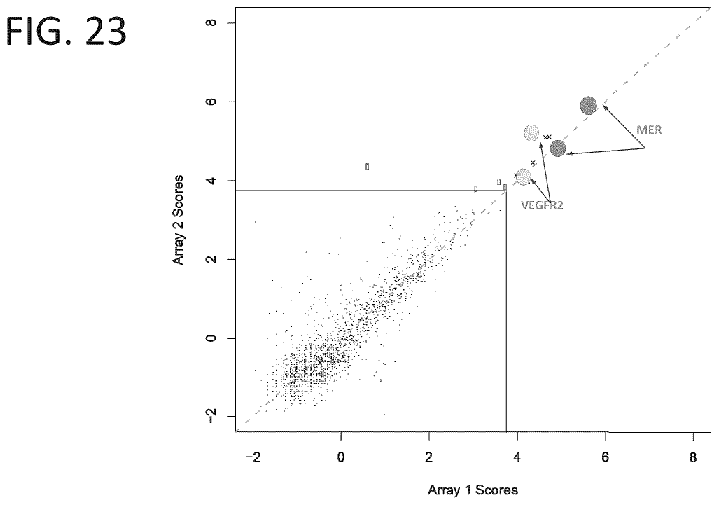

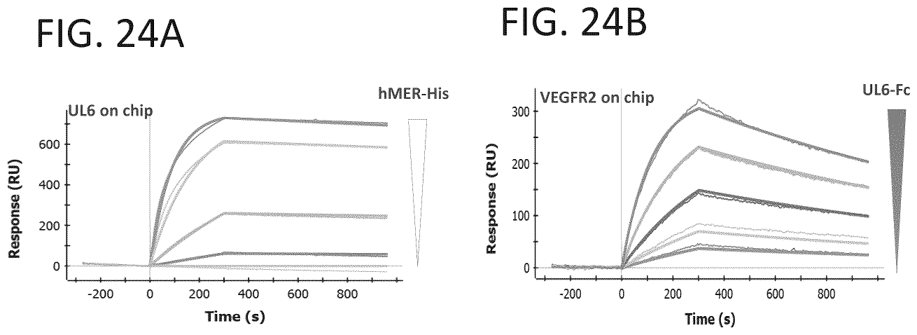

As shown in Examples 1 and 2 (FIGS. 1 and 19), the hCMV protein UL6 targets the human host receptors VEGFR2 and MERTK (MER) (FIG. 23). The protein microarray technology, data analysis, and scoring procedures used to generate FIG. 23 are described in Ramani et al., Anal Biochem, 420: 127-138, 2012. The interactions between hCMV UL6 and MERTK and VEGFR2 were confirmed using surface plasmon resonance (FIGS. 24A and 24B). SPR assays were performed using a XPR36 PROTEON™ Instrument (Biorad). In all measurements, proteins were immobilized on GLC sensor chips using standard amino coupling chemistry. Data were processed using the PROTEON™ Manager 3.1.0.6 software.

### B. UL6 Competition with Gas6 and Activation of Downstream Signaling

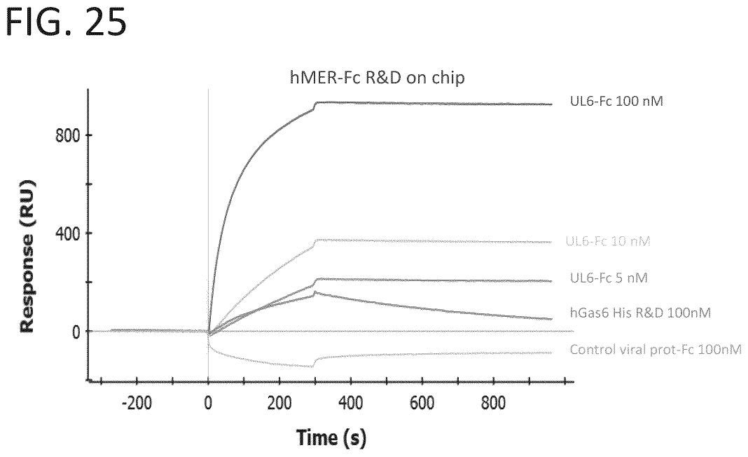

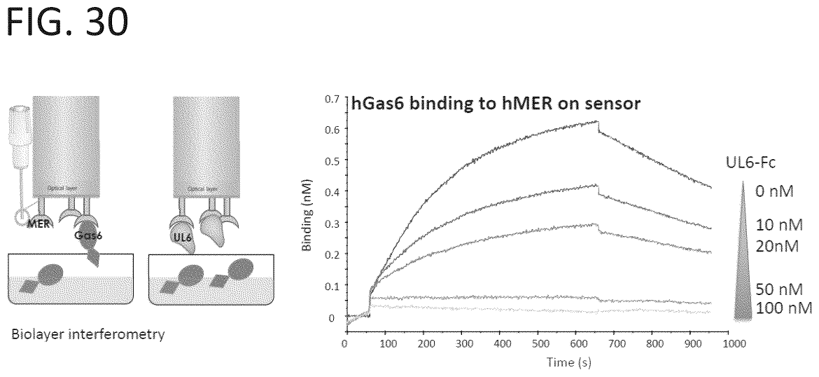

Growth arrest-specific 6 (Gas6) is a natural ligand of MERTK. UL6 demonstrated increased binding to MERTK relative to Gas6 in a SPR assay (FIG. 25), and efficiently competed with Gas6 for binding to MERTK in a biolayer interferometry (BLI) assay (FIG. 30). The Gas6 and MERTK proteins used for this analysis were purchased from R&D.

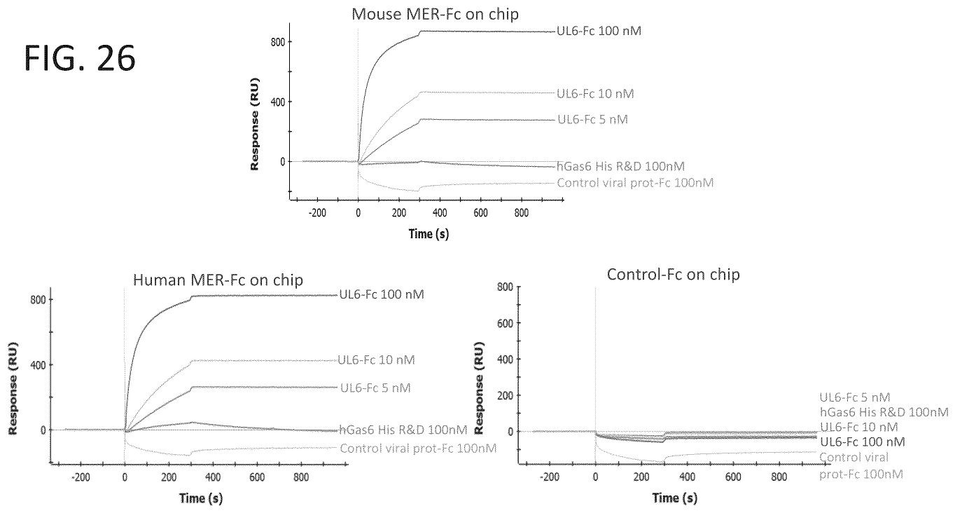

UL6 was found to bind human and mouse MERTK with similar affinity in an SPR assay (FIG. 26).

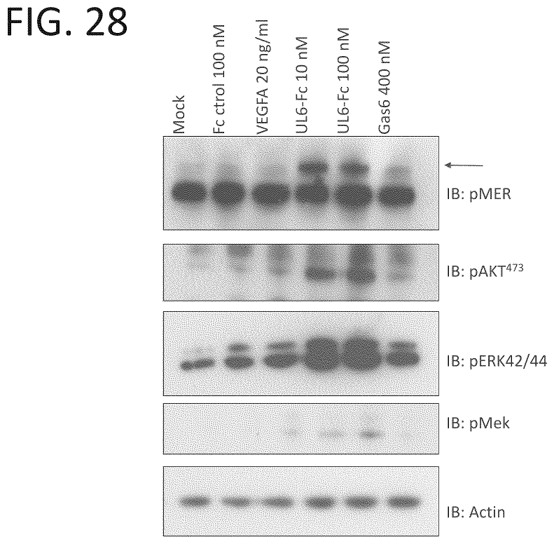

UL6 stimulation of HUVEC induced activation of MERTK and downstream signaling, as shown by increased phosphorylation of the relevant proteins (MERTK, AKT, ERK42/44, and MEK) (FIG. 28). UL6-induced activation of MERTK and downstream effectors in the pathway was higher than that observed upon stimulation with the natural ligand, Gas6 (FIG. 28). HUVEC cells were stimulated with the relevant proteins at 37° C., washed with cold PBS, and subsequently lysed in lysis buffer. Lysates were subjected to SDS-PAGE, and Western blotting was performed using the listed antibodies. All antibodies utilized were purchased from Cell Signaling Technologies. Gas6 and VEGFA were purchased from R&D.

### C. Promotion of Angiogenesis by UL6

HCMV is associated with several vasculopathies, including atherosclerosis, transplant vascular sclerosis, and glioblastoma. The hCMV protein UL7 is a relevant secreted factor that promotes angiogenesis in vitro (MacManiman et al., mBio, 5(6): e02035, 2014), and mediates leukocyte adhesion and inhibits proinflammatory cytokine production through unknown mechanisms (Engel et al., Immunol Cell Biol, 89(7): 753-766, 2011). The relationship of UL6 and MERTK to angiogenesis was investigated.

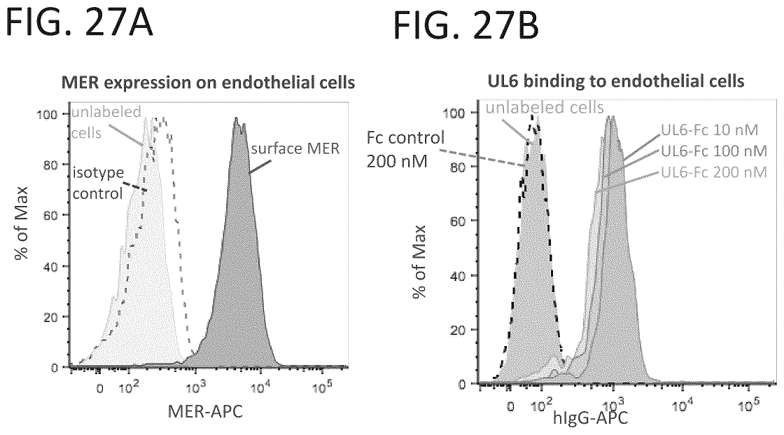

MERTK was found to be expressed on the surface of human umbilical vein endothelial cells (HUVECs) (FIG. 27A). UL6 was found to bind to MERTK on the surface of these cells (FIG. 27B).

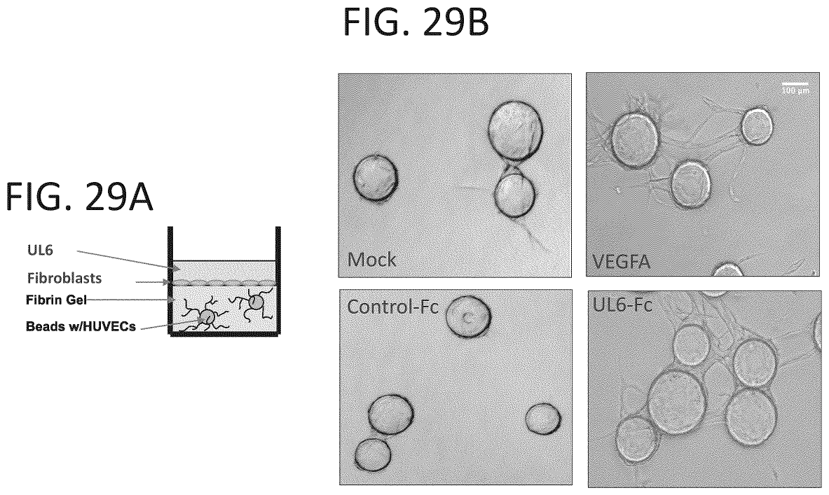

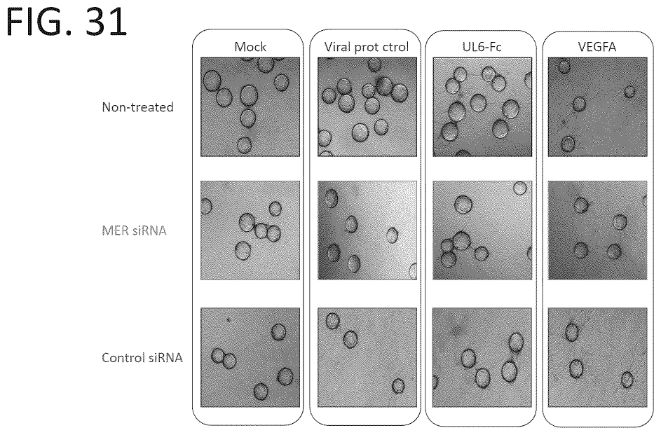

UL6 was found to promote angiogenesis in vitro. Tube formation assays were performed as shown in FIG. 29A: HUVEC cells were grown in 3D matrices (fibrin gel), and their ability to form capillary-like structures (or tubules) in the presence or absence of UL6 was assessed. UL6 was found to promote tube formation at a level comparable to the positive control VEGFA (FIG. 29B). UL6-mediated angiogenesis was found to be dependent on expression of MERTK on the endothelial cells: tube formation was decreased when cells were treated with MERTK siRNA (FIG. 31).

What is claimed is:

1. A method of identifying a modulator of the interaction between a protein of Table 1 and a protein of Table 2, the method comprising:
(a) providing a candidate modulator;
(b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and
(c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

2. A method of identifying a modulator of a downstream activity of a protein of Table 1, the method comprising:
(a) providing a candidate modulator;
(b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and
(c) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1.

3. A method of identifying a modulator of a downstream activity of a protein of Table 2, the method comprising:
(a) providing a candidate modulator;
(b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and
(c) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

4. The method of claim 1, wherein the increase or decrease in binding is at least 70%, as measured by a surface plasmon resonance (SPR) assay, a BLI assay, or an enzyme-linked immunosorbent assay (ELISA).

5. The method of claim 2 or 3, wherein the modulator is an inhibitor of the downstream activity of the protein of Table 1 or Table 2.

6. The method of claim 2 or 3, wherein the modulator is an activator of the downstream activity of the protein of Table 1 or Table 2.

7. The method of claim 2 or 3, wherein the change in the downstream activity is a decrease in the amount, strength, or duration of the downstream activity.

8. The method of claim 2 or 3, wherein the change in the downstream activity is an increase in the amount, strength, or duration of the downstream activity.

9. The method of any one of claims 2-8, wherein the downstream activity is infection of a cell by a member of the viral family Herpesviridae.

10. The method of claim 9, wherein infection is decreased in the presence of the modulator.

11. The method of claim 10, wherein infection is decreased by at least 40%, as measured in a viral infection assay or a viral entry assay.

12. The method of any one of claims 1-11, wherein the modulator is a small molecule, an antibody or antigen-binding fragment thereof, a peptide, a mimic, an antisense oligonucleotide, or a small interfering RNA (siRNA).

13. The method of claim 12, wherein the antigen-binding fragment is a bis-Fab, an Fv, a Fab, a Fab′-SH, a F(ab′)₂, a diabody, a linear antibody, an scFv, an ScFab, a VH domain, or a VHH domain.

14. The method of claim 12 or 13, wherein the antibody or antigen-binding fragment thereof binds the protein of Table 1.

15. The method of claim 12 or 13, wherein the antibody or antigen-binding fragment thereof binds the protein of Table 2.

16. A method of treating an individual having a herpes simplex virus 2 (HSV-2) infection comprising administering to the individual an effective amount of a CSPG5 antagonist, a PRRG2 antagonist, a UNC5D antagonist, or a PLB1 antagonist.

17. A method of decreasing HSV-2 infection in an individual comprising administering to the individual an effective amount of a CSPG5 antagonist, a PRRG2 antagonist, a UNC5D antagonist, or a PLB1 antagonist.

18. The method of claim 16 or 17, wherein:
(a) the CSPG5 antagonist results in a decrease in the binding of CSPG5 and the HSV-2 glycoprotein G (gG) protein relative to binding of the two proteins in the absence of the antagonist;
(b) the PRRG2 antagonist results in a decrease in the binding of PRRG2 and the HSV-2 gG protein relative to binding of the two proteins in the absence of the antagonist;
(c) the UNC5D antagonist results in a decrease in the binding of UNC5D and the HSV-2 gG protein relative to binding of the two proteins in the absence of the antagonist; or
(d) the PLB1 antagonist results in a decrease in the binding of PLB1 and the HSV-2 gD protein relative to binding of the two proteins in the absence of the antagonist.

19. The method of any one of claims 16-18, wherein the CSPG5 antagonist, PRRG2 antagonist, UNC5D antagonist, or PLB1 antagonist reduces the extent and/or severity of HSV-2 infection of the individual relative to infection in the absence of the CSPG5 antagonist, PRRG2 antagonist, UNC5D antagonist, or PLB1 antagonist, respectively.

20. The method of any one of claims 16-19, wherein the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

21. The method of claim 20, wherein the inhibitory nucleic acid is an antisense oligonucleotide (ASO) or a small interfering RNA (siRNA).

22. The method of claim 20, wherein the CSPG5 antagonist, PRRG2 antagonist, UNC5D antagonist, or PLB1 antagonist is a peptide.

23. The method of claim 20, wherein the CSPG5 antagonist, PRRG2 antagonist, UNC5D antagonist, or PLB1 antagonist is an antibody or antigen-binding fragment thereof.

24. The method of claim 23, wherein:
(a) the antibody or antigen-binding fragment thereof binds the HSV-2 gG protein and inhibits its binding to CSPG5, PRRG2, and/or UNC5D; or
(b) the antibody or antigen-binding fragment thereof binds the HSV-2 gD protein and inhibits its binding to PLB1.

25. The method of claim 23, wherein the antibody or antigen-binding fragment thereof binds CSPG5, PRRG2, UNC5D, or PLB1.

26. The method of claim 25, wherein:
(a) the antibody or antigen-binding fragment thereof inhibits the binding of CSPG5, PRRG2, or UNC5D to the HSV-2 gG protein; or
(b) the antibody or antigen-binding fragment thereof inhibits the binding of PLB1 to the HSV-2 gD protein.

27. The method of any one of claims 20 and 23-26, wherein the antigen-binding fragment is a bis-Fab, an Fv, a Fab, a Fab′-SH, a F(ab′)₂, a diabody, a linear antibody, an scFv, an scFab, a VH domain, or a VHH domain.

28. The method of any one of claims 16-27, wherein the individual has genital herpes or herpes simplex encephalitis.

29. The method of any one of claims 16-28, wherein the individual is a human.

30. A CSPG5 antagonist, a PRRG2 antagonist, a UNC5D antagonist, or a PLB1 antagonist for use as a medicament.

31. The CSPG5 antagonist, PRRG2 antagonist, UNC5D antagonist, or PLB1 antagonist for use of claim 30, wherein the medicament is for treating an HSV2 infection.

32. The CSPG5 antagonist, PRRG2 antagonist, UNC5D antagonist, or PLB1 antagonist for use of claim 30, wherein the medicament is for treating genital herpes or herpes simplex encephalitis.

33. The CSPG5 antagonist, PRRG2 antagonist, UNC5D antagonist, or PLB1 antagonist for use of any one of claims 30-32, wherein:
(a) the CSPG5 antagonist results in a decrease in the binding of CSPG5 and the HSV-2 glycoprotein G (gG) protein relative to binding of the two proteins in the absence of the antagonist;
(b) the PRRG2 antagonist results in a decrease in the binding of PRRG2 and the HSV-2 gG protein relative to binding of the two proteins in the absence of the antagonist;
(c) the UNC5D antagonist results in a decrease in the binding of UNC5D and the HSV-2 gG protein relative to binding of the two proteins in the absence of the antagonist; or
(d) the PLB1 antagonist results in a decrease in the binding of PLB1 and the HSV-2 gD protein relative to binding of the two proteins in the absence of the antagonist.

34. The CSPG5 antagonist, PRRG2 antagonist, UNC5D antagonist, or PLB1 antagonist for use of any one of claims 30-33, wherein the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

35. The CSPG5 antagonist, PRRG2 antagonist, UNC5D antagonist, or PLB1 antagonist for use of claim 34, wherein the inhibitory nucleic acid is an ASO or a siRNA.

36. The CSPG5 antagonist, PRRG2 antagonist, UNC5D antagonist, or PLB1 antagonist for use of claim 34, wherein the CSPG5 antagonist, PRRG2 antagonist, UNC5D antagonist, or PLB1 antagonist is a peptide.

37. The CSPG5 antagonist, PRRG2 antagonist, UNC5D antagonist, or PLB1 antagonist for use of claim 34, wherein the CSPG5 antagonist, PRRG2 antagonist, UNC5D antagonist, or PLB1 antagonist is an antibody or antigen-binding fragment thereof.

38. The CSPG5 antagonist, PRRG2 antagonist, UNC5D antagonist, or PLB1 antagonist for use of claim 37, wherein:
(a) the antibody or antigen-binding fragment thereof binds the HSV-2 gG protein and inhibits its binding to CSPG5, PRRG2, and/or UNC5D; or
(b) the antibody or antigen-binding fragment thereof binds the HSV-2 gD protein and inhibits its binding to PLB1.

39. The CSPG5 antagonist, PRRG2 antagonist, UNC5D antagonist, or PLB1 antagonist for use of claim 37, wherein the antibody or antigen-binding fragment thereof binds CSPG5, PRRG2, UNC5D, or PLB1.

40. The CSPG5 antagonist, PRRG2 antagonist, UNC5D antagonist, or PLB1 antagonist for use of claim 39, wherein:
(a) the antibody or antigen-binding fragment thereof inhibits the binding of CSPG5, PRRG2, or UNC5D to the HSV-2 gG protein; or
(b) the antibody or antigen-binding fragment thereof inhibits the binding of PVRL1 or PLB1 to the HSV-2 gD protein.

41. The CSPG5 antagonist, PRRG2 antagonist, UNC5D antagonist, or PLB1 antagonist for use of any one of claims 34 and 37-40, wherein the antigen-binding fragment is a bis-Fab, an Fv, a Fab, a Fab′-SH, a F(ab′)₂, a diabody, a linear antibody, an scFv, an scFab, a VH domain, or a VHH domain.

42. Use of a CSPG5 antagonist, a PRRG2 antagonist, a UNC5D antagonist, or a PLB1 antagonist in the manufacture of a medicament for treatment of an HSV-2 infection.

43. Use of a CSPG5 antagonist, a PRRG2 antagonist, a UNC5D antagonist, or a PLB1 antagonist in the manufacture of a medicament for treatment of genital herpes or herpes simplex encephalitis.

44. Use of a CSPG5 antagonist, a PRRG2 antagonist, a UNC5D antagonist, or a PLB1 antagonist in the manufacture of a medicament for reducing or preventing infection of a cell by HSV-2.

45. The use of any one of claims 42-44, wherein:
(a) the CSPG5 antagonist results in a decrease in the binding of CSPG5 and the HSV-2 glycoprotein G (gG) protein relative to binding of the two proteins in the absence of the antagonist;
(b) the PRRG2 antagonist results in a decrease in the binding of PRRG2 and the HSV-2 gG protein relative to binding of the two proteins in the absence of the antagonist;
(c) the UNC5D antagonist results in a decrease in the binding of UNC5D and the HSV-2 gG protein relative to binding of the two proteins in the absence of the antagonist; or
(d) the PLB1 antagonist results in a decrease in the binding of PLB1 and the HSV-2 gD protein relative to binding of the two proteins in the absence of the antagonist.

46. The use of any one of claims 42-45, wherein the CSPG5 antagonist, PRRG2 antagonist, UNC5D antagonist, or PLB1 antagonist is a small molecule, an antibody or antigen-binding fragment thereof, a peptide, a mimic, or an inhibitory nucleic acid.

47. The use of claim 46, wherein the inhibitory nucleic acid is an ASO or a siRNA.

48. The use of claim 46, wherein the CSPG5 antagonist, PRRG2 antagonist, UNC5D antagonist, or PLB1 antagonist is a peptide.

49. The use of claim 46, wherein the CSPG5 antagonist, PRRG2 antagonist, UNC5D antagonist, or PLB1 antagonist is an antibody or antigen-binding fragment thereof.

50. The use of claim 49, wherein:
(a) the antibody or antigen-binding fragment thereof binds the HSV-2 gG protein and inhibits its binding to CSPG5, PRRG2, and/or UNC5D; or
(b) the antibody or antigen-binding fragment thereof binds the HSV-2 gD protein and inhibits its binding to PLB1.

51. The use of claim 49, wherein the antibody or antigen-binding fragment thereof binds CSPG5, PRRG2, UNC5D, or PLB1.

52. The use of claim 51, wherein:
(a) the antibody or antigen-binding fragment thereof inhibits the binding of CSPG5, PRRG2, or UNC5D to the HSV-2 gG protein; or
(b) the antibody or antigen-binding fragment thereof inhibits the binding of PVRL1 or PLB1 to the HSV-2 gD protein.

53. The use of any one of claims 46 and 49-52, wherein the antigen-binding fragment is a bis-Fab, an Fv, a Fab, a Fab′-SH, a F(ab′)₂, a diabody, a linear antibody, an scFv, an scFab, a VH domain, or a VHH domain.

54. A method of treating an individual having a macacine alphaherpesvirus (MCHV) infection comprising administering to the individual an effective amount of a PILRA antagonist.

55. A method of decreasing MCHV infection in an individual comprising administering to the individual an effective amount of a PILRA antagonist.

56. The method of claim 54 or 55, wherein the PILRA antagonist results in a decrease in the binding of PILRA and the MCHV glycoprotein G (gG) protein relative to binding of the two proteins in the absence of the antagonist.

57. The method of any one of claims 54-56, wherein the PILRA antagonist reduces the extent and/or severity of MCHV infection of the individual relative to infection in the absence of the PILRA antagonist.

58. The method of any one of claims 54-57, wherein the PILRA antagonist is a small molecule, an antibody or antigen-binding fragment thereof, a peptide, a mimic, or an inhibitory nucleic acid.

59. The method of claim 58, wherein the inhibitory nucleic acid is an ASO or a siRNA.

60. The method of claim 58, wherein the PILRA antagonist is a peptide.

61. The method of claim 58, wherein the PILRA antagonist is an antibody or antigen-binding fragment thereof.

62. The method of claim 61, wherein the antibody or antigen-binding fragment thereof binds the MCHV gG protein and inhibits its binding to PILRA.

63. The method of claim 61, wherein the antibody or antigen-binding fragment thereof binds PILRA.

64. The method of claim 63, wherein the antibody or antigen-binding fragment thereof inhibits the binding of PILRA to the MCHV gG protein.

65. The method of any one of claims 58 and 61-64, wherein the antigen-binding fragment is a bis-Fab, an Fv, a Fab, a Fab′-SH, a F(ab′)₂, a diabody, a linear antibody, an scFv, an scFab, a VH domain, or a VHH domain.

66. The method of any one of claims 54-65, wherein the individual has a zoonotic MCHV infection.

67. The method of any one of claims 54-66, wherein the individual is a human.

68. A PILRA antagonist for use as a medicament, wherein the medicament is for treating an MCHV infection.

69. The PILRA antagonist for use of claim 68, wherein the medicament is for treating a zoonotic MCHV infection.

70. The PILRA antagonist for use of claim 68 or 69, wherein the PILRA antagonist results in a decrease in the binding of PILRA and the MCHV glycoprotein G (gG) protein relative to binding of the two proteins in the absence of the antagonist.

71. The PILRA antagonist for use of any one of claims 68-70, wherein the PILRA antagonist is a small molecule, an antibody or antigen-binding fragment thereof, a peptide, a mimic, or an inhibitory nucleic acid.

72. The PILRA antagonist for use of claim 71, wherein the inhibitory nucleic acid is an ASO or a siRNA.

73. The PILRA antagonist for use of claim 71, wherein the PILRA antagonist is a peptide.

74. The PILRA antagonist for use of claim 71, wherein the PILRA antagonist is an antibody or antigen-binding fragment thereof.

75. The PILRA antagonist for use of claim 74, wherein the antibody or antigen-binding fragment thereof binds the MCHV gG protein and inhibits its binding to PILRA.

76. The PILRA antagonist for use of claim 74, wherein the antibody or antigen-binding fragment thereof binds PILRA.

77. The PILRA antagonist for use of claim 76, wherein the antibody or antigen-binding fragment thereof inhibits the binding of PILRA to the MCHV gG protein.

78. The PILRA antagonist for use of any one of claims 71 and 74-77, wherein the antigen-binding fragment is a bis-Fab, an Fv, a Fab, a Fab′-SH, a F(ab′)₂, a diabody, a linear antibody, an scFv, an scFab, a VH domain, or a VHH domain.

79. Use of a PILRA antagonist in the manufacture of a medicament for treatment of a MCHV infection.

80. Use of a PILRA antagonist in the manufacture of a medicament for treatment of a zoonotic MCHV infection.

81. Use of a PILRA antagonist in the manufacture of a medicament for reducing or preventing infection of a cell by MCHV.

82. The use of any one of claims 79-81, wherein the PILRA antagonist results in a decrease in the binding of PILRA and the MCHV glycoprotein G (gG) protein relative to binding of the two proteins in the absence of the antagonist.

83. The use of any one of claims 79-82, wherein the PILRA antagonist is a small molecule, an antibody or antigen-binding fragment thereof, a peptide, a mimic, or an inhibitory nucleic acid.

84. The use of claim 83, wherein the inhibitory nucleic acid is an ASO or a siRNA.

85. The use of claim 83, wherein the PILRA antagonist is a peptide.

86. The use of claim 83, wherein the PILRA antagonist is an antibody or antigen-binding fragment thereof.

87. The use of claim 86, wherein the antibody or antigen-binding fragment thereof binds the MCHV gG protein and inhibits its binding to PILRA.

88. The use of claim 86, wherein the antibody or antigen-binding fragment thereof binds PILRA.

89. The use of claim 88, wherein the antibody or antigen-binding fragment thereof inhibits the binding of PILRA to the MCHV gG protein.

90. The use of any one of claims 83 and 86-89, wherein the antigen-binding fragment is a bis-Fab, an Fv, a Fab, a Fab′-SH, a F(ab′)₂, a diabody, a linear antibody, an scFv, an scFab, a VH domain, or a VHH domain.

91. A method of treating an individual having a human cytomegalovirus (HCMV) infection comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist.

92. A method of decreasing HCMV infection in an individual comprising administering to the individual an effective amount of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist.

93. The method of claim 91 or 92, wherein:
(a) the VEGFR2 antagonist results in a decrease in the binding of VEGFR2 and the HCMV UL6 protein relative to binding of the two proteins in the absence of the antagonist;
(b) the MERTK antagonist results in a decrease in the binding of MERTK and the HCMV UL6 protein relative to binding of the two proteins in the absence of the antagonist;
(c) the PDGFRa antagonist results in a decrease in the binding of PDGFRa and the HCMV UL6 protein relative to binding of the two proteins in the absence of the antagonist;
(d) the KIRREL2 antagonist results in a decrease in the binding of KIRREL2 and the HCMV UL6 protein relative to binding of the two proteins in the absence of the antagonist;
(e) the LILRB5 antagonist results in a decrease in the binding of LILRB5 and the HCMV UL9 protein relative to binding of the two proteins in the absence of the antagonist;
(f) the ULBP1 antagonist results in a decrease in the binding of ULBP1 and the HCMV UL9 protein relative to binding of the two proteins in the absence of the antagonist;
(g) the KIR2DL3 antagonist results in a decrease in the binding of KIR2DL3 and the HCMV UL9 protein relative to binding of the two proteins in the absence of the antagonist;
(h) the KIR2DS1 antagonist results in a decrease in the binding of KIR2DS1 and the HCMV UL9 protein relative to binding of the two proteins in the absence of the antagonist;
(i) the KIR2DS2 antagonist results in a decrease in the binding of KIR2DS2 and the HCMV UL9 protein relative to binding of the two proteins in the absence of the antagonist;
(j) the KIR2DS4 antagonist results in a decrease in the binding of KIR2DS4 and the HCMV UL9 protein relative to binding of the two proteins in the absence of the antagonist;
(k) the KIR2DS5 antagonist results in a decrease in the binding of KIR2DS5 and the HCMV UL9 protein relative to binding of the two proteins in the absence of the antagonist;
(l) the KIR2DL1 antagonist results in a decrease in the binding of KIR2DL1 and the HCMV UL9 protein relative to binding of the two proteins in the absence of the antagonist;
(m) the KIR3DL1 antagonist results in a decrease in the binding of KIR3DL1 and the HCMV UL9 protein relative to binding of the two proteins in the absence of the antagonist;
(n) the PRRG2 antagonist results in a decrease in the binding of PRRG2 and the HCMV UL142 protein relative to binding of the two proteins in the absence of the antagonist;
(o) the KLRAP1 antagonist results in a decrease in the binding of KLRAP1 and the HCMV UL144 protein relative to binding of the two proteins in the absence of the antagonist; or
(p) the SGCA antagonist results in a decrease in the binding of SGCA and the HCMV RL10 protein relative to binding of the two proteins in the absence of the antagonist.

94. The method of any one of claims 91-93, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist reduces the extent and/or severity of HCMV infection of the individual relative to infection in the absence of the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist, respectively.

95. The method of any one of claims 91-94, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a small molecule, an antibody or antigen-binding fragment thereof, a peptide, a mimic, or an inhibitory nucleic acid.

96. The method of claim 95, wherein the inhibitory nucleic acid is an ASO or a siRNA.

97. The method of claim 95, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

98. The method of claim 95, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof.

99. The method of claim 98, wherein:
(a) the antibody or antigen-binding fragment thereof binds the HCMV UL6 protein and inhibits its binding to VEGFR2, MERTK, PDGFRa, and/or KIRREL2;
(b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1;
(c) the antibody or antigen-binding fragment thereof binds the HCMV UL142 protein and inhibits its binding to PRRG2;
(d) the antibody or antigen-binding fragment thereof binds the HCMV UL144 protein and inhibits its binding to KLRAP1; or
(e) the antibody or antigen-binding fragment thereof binds the HCMV RL10 protein and inhibits its binding to SGCA.

100. The method of claim 98, wherein the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA.

101. The method of claim 100, wherein:
(a) the antibody or antigen-binding fragment thereof inhibits the binding of VEGFR2, MERTK, PDGFRa, or KIRREL2 to the HCMV UL6 protein;
(b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein;
(c) the antibody or antigen-binding fragment thereof inhibits the binding of PRRG2 to the HCMV UL142 protein;
(d) the antibody or antigen-binding fragment thereof inhibits the binding of KLRAP1 to the HCMV UL144 protein; or
(e) the antibody or antigen-binding fragment thereof inhibits the binding of SGCA to the HCMV RL10 protein.

102. The method of any one of claims 95 and 98-101, wherein the antigen-binding fragment is a bis-Fab, an Fv, a Fab, a Fab′-SH, a F(ab′)₂, a diabody, a linear antibody, an scFv, an scFab, a VH domain, or a VHH domain.

103. The method of any one of claims 91-102, wherein the HCMV infection is congenital.

104. The method of any one of claims 91-103, wherein the individual has CMV-related allograft rejection.

105. The method of any one of claims 91-104, wherein the individual is a human.

106. A KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist for use as a medicament.

107. A VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use as a medicament, wherein the medicament is for treating a HCMV infection.

108. A VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use as a medicament, wherein the medicament is for treating CMV-related allograft rejection.

109. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 107 or 108, wherein:
(a) the VEGFR2 antagonist results in a decrease in the binding of VEGFR2 and the HCMV UL6 protein relative to binding of the two proteins in the absence of the antagonist;
(b) the MERTK antagonist results in a decrease in the binding of MERTK and the HCMV UL6 protein relative to binding of the two proteins in the absence of the antagonist;
(c) the PDGFRa antagonist results in a decrease in the binding of PDGFRa and the HCMV UL6 protein relative to binding of the two proteins in the absence of the antagonist;
(d) the KIRREL2 antagonist results in a decrease in the binding of KIRREL2 and the HCMV UL6 protein relative to binding of the two proteins in the absence of the antagonist;
(e) the LILRB5 antagonist results in a decrease in the binding of LILRB5 and the HCMV UL9 protein relative to binding of the two proteins in the absence of the antagonist;
(f) the ULBP1 antagonist results in a decrease in the binding of ULBP1 and the HCMV UL9 protein relative to binding of the two proteins in the absence of the antagonist;
(g) the KIR2DL3 antagonist results in a decrease in the binding of KIR2DL3 and the HCMV UL9 protein relative to binding of the two proteins in the absence of the antagonist;
(h) the KIR2DS1 antagonist results in a decrease in the binding of KIR2DS1 and the HCMV UL9 protein relative to binding of the two proteins in the absence of the antagonist;
(i) the KIR2DS2 antagonist results in a decrease in the binding of KIR2DS2 and the HCMV UL9 protein relative to binding of the two proteins in the absence of the antagonist;
(j) the KIR2DS4 antagonist results in a decrease in the binding of KIR2DS4 and the HCMV UL9 protein relative to binding of the two proteins in the absence of the antagonist;
(k) the KIR2DS5 antagonist results in a decrease in the binding of KIR2DS5 and the HCMV UL9 protein relative to binding of the two proteins in the absence of the antagonist;
(l) the KIR2DL1 antagonist results in a decrease in the binding of KIR2DL1 and the HCMV UL9 protein relative to binding of the two proteins in the absence of the antagonist;
(m) the KIR3DL1 antagonist results in a decrease in the binding of KIR3DL1 and the HCMV UL9 protein relative to binding of the two proteins in the absence of the antagonist;
(n) the PRRG2 antagonist results in a decrease in the binding of PRRG2 and the HCMV UL142 protein relative to binding of the two proteins in the absence of the antagonist;
(o) the KLRAP1 antagonist results in a decrease in the binding of KLRAP1 and the HCMV UL144 protein relative to binding of the two proteins in the absence of the antagonist; or
(p) the SGCA antagonist results in a decrease in the binding of SGCA and the HCMV RL10 protein relative to binding of the two proteins in the absence of the antagonist.

110. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of any one of claims 107-109, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist reduces the extent and/or severity of HCMV infection of the individual relative to infection in the absence of the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist, respectively.

111. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 110, wherein the inhibitory nucleic acid is an ASO or a siRNA.

112. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 110, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

113. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 110, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof.

114. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 113, wherein:
(a) the antibody or antigen-binding fragment thereof binds the HCMV UL6 protein and inhibits its binding to VEGFR2, MERTK, PDGFRa, and/or KIRREL2;
(b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1;
(c) the antibody or antigen-binding fragment thereof binds the HCMV UL142 protein and inhibits its binding to PRRG2;
(d) the antibody or antigen-binding fragment thereof binds the HCMV UL144 protein and inhibits its binding to KLRAP1 and/or BTLA; or
(e) the antibody or antigen-binding fragment thereof binds the HCMV RL10 protein and inhibits its binding to SGCA.

115. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 113, wherein the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA.

116. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of claim 115, wherein:
(a) the antibody or antigen-binding fragment thereof inhibits the binding of VEGFR2, MERTK, PDGFRa, or KIRREL2 to the HCMV UL6 protein;
(b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein;
(c) the antibody or antigen-binding fragment thereof inhibits the binding of PRRG2 to the HCMV UL142 protein;
(d) the antibody or antigen-binding fragment thereof inhibits the binding of KLRAP1 or BTLA to the HCMV UL144 protein; or
(e) the antibody or antigen-binding fragment thereof inhibits the binding of SGCA to the HCMV RL10 protein.

117. The VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist for use of any one of claims 110 and 113-116, wherein the antigen-binding fragment is a bis-Fab, an Fv, a Fab, a Fab′-SH, a F(ab′)₂, a diabody, a linear antibody, an scFv, an scFab, a VH domain, or a VHH domain.

118. Use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for treatment of an HCMV infection.

119. Use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for treatment of CMV-related allograft rejection.

120. Use of a VEGFR2 antagonist, a MERTK antagonist, a PDGFRa antagonist, a KIRREL2 antagonist, a LILRB5 antagonist, a ULBP1 antagonist, a KIR2DL3 antagonist, a KIR2DS1 antagonist, a KIR2DS2 antagonist, a KIR2DS4 antagonist, a KIR2DS5 antagonist, a KIR2DL1 antagonist, a KIR3DL1 antagonist, a PRRG2 antagonist, a KLRAP1 antagonist, or a SGCA antagonist in the manufacture of a medicament for reducing or preventing infection of a cell by HCMV.

121. The use of any one of claims 118-120, wherein:
(a) the VEGFR2 antagonist results in a decrease in the binding of VEGFR2 and the HCMV UL6 protein relative to binding of the two proteins in the absence of the antagonist;
(b) the MERTK antagonist results in a decrease in the binding of MERTK and the HCMV UL6 protein relative to binding of the two proteins in the absence of the antagonist;
(c) the PDGFRa antagonist results in a decrease in the binding of PDGFRa and the HCMV UL6 protein relative to binding of the two proteins in the absence of the antagonist;
(d) the KIRREL2 antagonist results in a decrease in the binding of KIRREL2 and the HCMV UL6 protein relative to binding of the two proteins in the absence of the antagonist;
(e) the LILRB5 antagonist results in a decrease in the binding of LILRB5 and the HCMV UL9 protein relative to binding of the two proteins in the absence of the antagonist;
(f) the ULBP1 antagonist results in a decrease in the binding of ULBP1 and the HCMV UL9 protein relative to binding of the two proteins in the absence of the antagonist;
(g) the KIR2DL3 antagonist results in a decrease in the binding of KIR2DL3 and the HCMV UL9 protein relative to binding of the two proteins in the absence of the antagonist;
(h) the KIR2DS1 antagonist results in a decrease in the binding of KIR2DS1 and the HCMV UL9 protein relative to binding of the two proteins in the absence of the antagonist;
(i) the KIR2DS2 antagonist results in a decrease in the binding of KIR2DS2 and the HCMV UL9 protein relative to binding of the two proteins in the absence of the antagonist;
(j) the KIR2DS4 antagonist results in a decrease in the binding of KIR2DS4 and the HCMV UL9 protein relative to binding of the two proteins in the absence of the antagonist;
(k) the KIR2DS5 antagonist results in a decrease in the binding of KIR2DS5 and the HCMV UL9 protein relative to binding of the two proteins in the absence of the antagonist;
(l) the KIR2DL1 antagonist results in a decrease in the binding of KIR2DL1 and the HCMV UL9 protein relative to binding of the two proteins in the absence of the antagonist;
(m) the KIR3DL1 antagonist results in a decrease in the binding of KIR3DL1 and the HCMV UL9 protein relative to binding of the two proteins in the absence of the antagonist;
(n) the PRRG2 antagonist results in a decrease in the binding of PRRG2 and the HCMV UL142 protein relative to binding of the two proteins in the absence of the antagonist;
(o) the KLRAP1 antagonist results in a decrease in the binding of KLRAP1 and the HCMV UL144 protein relative to binding of the two proteins in the absence of the antagonist; or
(p) the SGCA antagonist results in a decrease in the binding of SGCA and the HCMV RL10 protein relative to binding of the two proteins in the absence of the antagonist.

122. The use of any one of claims 118-121, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a small molecule, an antibody or antigen-binding fragment thereof, a peptide, a mimic, or an inhibitory nucleic acid.

123. The use of claim 122, wherein the inhibitory nucleic acid is an ASO or a siRNA.

124. The use of claim 122, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is a peptide.

125. The use of claim 122, wherein the VEGFR2 antagonist, MERTK antagonist, PDGFRa antagonist, KIRREL2 antagonist, LILRB5 antagonist, ULBP1 antagonist, KIR2DL3 antagonist, KIR2DS1 antagonist, KIR2DS2 antagonist, KIR2DS4 antagonist, KIR2DS5 antagonist, KIR2DL1 antagonist, KIR3DL1 antagonist, PRRG2 antagonist, KLRAP1 antagonist, or SGCA antagonist is an antibody or antigen-binding fragment thereof.

126. The use of claim 125, wherein:
(a) the antibody or antigen-binding fragment thereof binds the HCMV UL6 protein and inhibits its binding to VEGFR2, MERTK, PDGFRa, and/or KIRREL2;
(b) the antibody or antigen-binding fragment thereof binds the HCMV UL9 protein and inhibits its binding to LILRB5, ULBP1, KIR2DL3, KIR2DS1, KIR2DS2, KIR2DS4, KIR2DS5, KIR2DL1, and/or KIR3DL1;
(c) the antibody or antigen-binding fragment thereof binds the HCMV UL142 protein and inhibits its binding to PRRG2;
(d) the antibody or antigen-binding fragment thereof binds the HCMV UL144 protein and inhibits its binding to KLRAP1 and/or BTLA; or
(e) the antibody or antigen-binding fragment thereof binds the HCMV RL10 protein and inhibits its binding to SGCA.

127. The use of claim 125, wherein the antibody or antigen-binding fragment thereof binds VEGFR2, MERTK, PDGFRa, KIRREL2, LILRB5, ULBP1, KIR2DL3, KIR2DS1, KIR2DS2, KIR2DS4, KIR2DS5, KIR2DL1, KIR3DL1, PRRG2, KLRAP1, or SGCA.

128. The use of claim 127, wherein:
(a) the antibody or antigen-binding fragment thereof inhibits the binding of VEGFR2, MERTK, PDGFRa, or KIRREL2 to the HCMV UL6 protein;
(b) the antibody or antigen-binding fragment thereof inhibits the binding of LILRB5, ULBP1, KIR2DL3, KIR2DS1, KIR2DS2, KIR2DS4, KIR2DS5, KIR2DL1, or KIR3DL1 to the HCMV UL9 protein;
(c) the antibody or antigen-binding fragment thereof inhibits the binding of PRRG2 to the HCMV UL142 protein;
(d) the antibody or antigen-binding fragment thereof inhibits the binding of KLRAP1 or BTLA to the HCMV UL144 protein; or
(e) the antibody or antigen-binding fragment thereof inhibits the binding of SGCA to the HCMV RL10 protein.

129. The use of any one of claims 122 and 125-128, wherein the antigen-binding fragment is a bis-Fab, an Fv, a Fab, a Fab′-SH, a F(ab′)₂, a diabody, a linear antibody, an scFv, an scFab, a VH domain, or a VHH domain.

130. A method of treating an individual having a Varicella zoster virus (VZV) infection comprising administering to the individual an effective amount of an ICAM1 antagonist, a MUSK antagonist, a HAVCR1 antagonist, a MOG antagonist, or a KIAA0319L antagonist.

131. A method of decreasing VZV infection in an individual comprising administering to the individual an effective amount of an ICAM1 antagonist, a MUSK antagonist, a HAVCR1 antagonist, a MOG antagonist, or a KIAA0319L antagonist.

132. The method of claim 130 or 131, wherein:
(a) the ICAM1 antagonist results in a decrease in the binding of ICAM1 and the VZV glycoprotein C (gC) protein relative to binding of the two proteins in the absence of the antagonist;
(b) the MUSK antagonist results in a decrease in the binding of MUSK and the VZV glycoprotein B (gB) protein relative to binding of the two proteins in the absence of the antagonist;
(c) the HAVCR1 antagonist results in a decrease in the binding of HAVCR1 and the VZV gB protein relative to binding of the two proteins in the absence of the antagonist;
(d) the MOG antagonist results in a decrease in the binding of MOG and the VZV glycoprotein I (gI) protein relative to binding of the two proteins in the absence of the antagonist; or
(e) the KIAA0319L antagonist results in a decrease in the binding of KIAA0319L and the VZV gI protein relative to binding of the two proteins in the absence of the antagonist.

133. The method of any one of claims 130-132, wherein the ICAM1 antagonist, MUSK antagonist, HAVCR1 antagonist, MOG antagonist, or KIAA0319L antagonist reduces the extent and/or severity of VZV infection of the individual relative to infection in the absence of the ICAM1 antagonist, MUSK antagonist, HAVCR1 antagonist, MOG antagonist, or KIAA0319L antagonist, respectively.

134. The method of any one of claims 130-133, wherein the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

135. The method of claim 134, wherein the inhibitory nucleic acid is an ASO or a siRNA.

136. The method of claim 134, wherein the ICAM1 antagonist, MUSK antagonist, HAVCR1 antagonist, MOG antagonist, or KIAA0319L antagonist is a peptide.

137. The method of claim 134, wherein the ICAM1 antagonist, MUSK antagonist, HAVCR1 antagonist, MOG antagonist, or KIAA0319L antagonist is an antibody or antigen-binding fragment thereof.

138. The method of claim 137, wherein:
(a) the antibody or antigen-binding fragment thereof binds the VZV gC protein and inhibits its binding to ICAM1;
(b) the antibody or antigen-binding fragment thereof binds the VZV gB protein and inhibits its binding to MUSK and/or HAVCR1; or
(c) the antibody or antigen-binding fragment thereof binds the VZV gI protein and inhibits its binding to MOG and/or KIAA0319L.

139. The method of claim 137, wherein the antibody or antigen-binding fragment thereof binds ICAM1, MUSK, HAVCR1, MOG, or KIAA0319L.

140. The method of claim 139, wherein:
(a) the antibody or antigen-binding fragment thereof inhibits the binding of ICAM1 to the VZV gC protein;
(b) the antibody or antigen-binding fragment thereof inhibits the binding of MUSK or HAVCR1 to the VZV gB protein; or
(c) the antibody or antigen-binding fragment thereof inhibits the binding of MOG or KIAA0319L to the VZV gI protein.

141. The method of any one of claims 134 and 137-140, wherein the antigen-binding fragment is a bis-Fab, an Fv, a Fab, a Fab′-SH, a F(ab′)₂, a diabody, a linear antibody, an scFv, an scFab, a VH domain, or a VHH domain.

142. The method of any one of claims 130-141, wherein the individual has chicken pox or shingles.

143. The method of any one of claims 130-142, wherein the individual is a human.

144. A MOG antagonist, a MUSK antagonist, or a KIAA0319L antagonist for use as a medicament.

145. An ICAM1 antagonist, MUSK antagonist, HAVCR1 antagonist, MOG antagonist, or KIAA0319L antagonist for use as a medicament, wherein the medicament is for treating a VZV infection.

146. An ICAM1 antagonist, MUSK antagonist, HAVCR1 antagonist, MOG antagonist, or KIAA0319L antagonist for use as a medicament, wherein the medicament is for treating chicken pox or shingles.

147. The ICAM1 antagonist, MUSK antagonist, HAVCR1 antagonist, MOG antagonist, or KIAA0319L antagonist for use of claim 145 or 146, wherein:
(a) the ICAM1 antagonist results in a decrease in the binding of ICAM1 and the VZV glycoprotein C (gC) protein relative to binding of the two proteins in the absence of the antagonist;
(b) the MUSK antagonist results in a decrease in the binding of MUSK and the VZV glycoprotein B (gB) protein relative to binding of the two proteins in the absence of the antagonist;
(c) the HAVCR1 antagonist results in a decrease in the binding of HAVCR1 and the VZV gB protein relative to binding of the two proteins in the absence of the antagonist;
(d) the MOG antagonist results in a decrease in the binding of MOG and the VZV glycoprotein I (gI) protein relative to binding of the two proteins in the absence of the antagonist; or
(e) the KIAA0319L antagonist results in a decrease in the binding of KIAA0319L and the VZV gI protein relative to binding of the two proteins in the absence of the antagonist.

148. The ICAM1 antagonist, MUSK antagonist, HAVCR1 antagonist, MOG antagonist, or KIAA0319L antagonist for use of any one of claims 145-147, wherein the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

149. The ICAM1 antagonist, MUSK antagonist, HAVCR1 antagonist, MOG antagonist, or KIAA0319L antagonist for use of claim 148, wherein the inhibitory nucleic acid is an ASO or a siRNA.

150. The ICAM1 antagonist, MUSK antagonist, HAVCR1 antagonist, MOG antagonist, or KIAA0319L antagonist for use of claim 148, wherein the ICAM1 antagonist, MUSK antagonist, HAVCR1 antagonist, MOG antagonist, or KIAA0319L antagonist is a peptide.

151. The ICAM1 antagonist, MUSK antagonist, HAVCR1 antagonist, MOG antagonist, or KIAA0319L antagonist for use of claim 148, wherein the ICAM1 antagonist, MUSK antagonist, HAVCR1 antagonist, MOG antagonist, or KIAA0319L antagonist is an antibody or antigen-binding fragment thereof.

152. The ICAM1 antagonist, MUSK antagonist, HAVCR1 antagonist, MOG antagonist, or KIAA0319L antagonist for use of claim 151, wherein:
(a) the antibody or antigen-binding fragment thereof binds the VZV gC protein and inhibits its binding to ICAM1;
(b) the antibody or antigen-binding fragment thereof binds the VZV gB protein and inhibits its binding to MUSK and/or HAVCR1; or
(c) the antibody or antigen-binding fragment thereof binds the VZV gI protein and inhibits its binding to MOG and/or KIAA0319L.

153. The ICAM1 antagonist, MUSK antagonist, HAVCR1 antagonist, MOG antagonist, or KIAA0319L antagonist for use of claim 152, wherein the antibody or antigen-binding fragment thereof binds ICAM1, MUSK, HAVCR1, MOG, or KIAA0319L.

154. The ICAM1 antagonist, MUSK antagonist, HAVCR1 antagonist, MOG antagonist, or KIAA0319L antagonist for use of claim 153, wherein:
(a) the antibody or antigen-binding fragment thereof inhibits the binding of ICAM1 to the VZV gC protein;
(b) the antibody or antigen-binding fragment thereof inhibits the binding of MUSK or HAVCR1 to the VZV gB protein; or
(c) the antibody or antigen-binding fragment thereof inhibits the binding of MOG or KIAA0319L to the VZV gI protein.

155. The ICAM1 antagonist, MUSK antagonist, HAVCR1 antagonist, MOG antagonist, or KIAA0319L antagonist for use of any one of claims 148 and 151-154, wherein the antigen-binding fragment is a bis-Fab, an Fv, a Fab, a Fab′-SH, a F(ab′)₂, a diabody, a linear antibody, an scFv, an scFab, a VH domain, or a VHH domain.

156. Use of an ICAM1 antagonist, a MUSK antagonist, a HAVCR1 antagonist, a MOG antagonist, or a KIAA0319L antagonist in the manufacture of a medicament for treatment of a VZV infection.

157. Use of an ICAM1 antagonist, a MUSK antagonist, a HAVCR1 antagonist, a MOG antagonist, or a KIAA0319L antagonist in the manufacture of a medicament for treatment of chicken pox or shingles.

158. Use of an ICAM1 antagonist, a MUSK antagonist, a HAVCR1 antagonist, a MOG antagonist, or a KIAA0319L antagonist in the manufacture of a medicament for reducing or preventing infection of a cell by VZV.

159. The use of any one of claims 156-158, wherein:
(a) the ICAM1 antagonist results in a decrease in the binding of ICAM1 and the VZV glycoprotein C (gC) protein relative to binding of the two proteins in the absence of the antagonist;
(b) the MUSK antagonist results in a decrease in the binding of MUSK and the VZV glycoprotein B (gB) protein relative to binding of the two proteins in the absence of the antagonist;
(c) the HAVCR1 antagonist results in a decrease in the binding of HAVCR1 and the VZV gB protein relative to binding of the two proteins in the absence of the antagonist;
(d) the MOG antagonist results in a decrease in the binding of MOG and the VZV glycoprotein I (gI) protein relative to binding of the two proteins in the absence of the antagonist; or
(e) the KIAA0319L antagonist results in a decrease in the binding of KIAA0319L and the VZV gI protein relative to binding of the two proteins in the absence of the antagonist.

160. The use of any one of claims 156-159, wherein the ICAM1 antagonist, MUSK antagonist, HAVCR1 antagonist, MOG antagonist, or KIAA0319L antagonist is a small molecule, an antibody or antigen-binding fragment thereof, a peptide, a mimic, or an inhibitory nucleic acid.

161. The use of claim 160, wherein the inhibitory nucleic acid is an ASO or a siRNA.

162. The use of claim 160, wherein the ICAM1 antagonist, MUSK antagonist, HAVCR1 antagonist, MOG antagonist, or KIAA0319L antagonist is a peptide.

163. The use of claim 160, wherein the ICAM1 antagonist, MUSK antagonist, HAVCR1 antagonist, MOG antagonist, or KIAA0319L antagonist is an antibody or antigen-binding fragment thereof.

164. The use of claim 163, wherein:
(a) the antibody or antigen-binding fragment thereof binds the VZV gC protein and inhibits its binding to ICAM1;
(b) the antibody or antigen-binding fragment thereof binds the VZV gB protein and inhibits its binding to MUSK and/or HAVCR1; or
(c) the antibody or antigen-binding fragment thereof binds the VZV gI protein and inhibits its binding to MOG and/or KIAA0319L.

165. The use of claim 163, wherein the antibody or antigen-binding fragment thereof binds ICAM1, MUSK, HAVCR1, MOG, or KIAA0319L.

166. The use of claim 165, wherein:
(a) the antibody or antigen-binding fragment thereof inhibits the binding of ICAM1 to the VZV gC protein;
(b) the antibody or antigen-binding fragment thereof inhibits the binding of MUSK or HAVCR1 to the VZV gB protein; or
(c) the antibody or antigen-binding fragment thereof inhibits the binding of MOG or KIAA0319L to the VZV gI protein.

167. The use of any one of claims 160 and 163-166, wherein the antigen-binding fragment is a bis-Fab, an Fv, a Fab, a Fab′-SH, a F(ab′)₂, a diabody, a linear antibody, an scFv, an scFab, a VH domain, or a VHH domain.

168. A method of treating an individual having a human herpesvirus 8 (HHV8) infection comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

169. A method of decreasing HHV8 infection in an individual comprising administering to the individual an effective amount of a KLRAP1 antagonist, a LILRB1 antagonist, a CLEC4G antagonist, a FLRT1 antagonist, a FLRT2 antagonist, or a FLRT3 antagonist.

170. The method of claim 168 or 169, wherein:
(a) the KLRAP1 antagonist results in a decrease in the binding of KLRAP1 and the HHV8 K14 protein relative to binding of the two proteins in the absence of the antagonist;
(b) the LILRB1 antagonist results in a decrease in the binding of LILRB1 and the HHV8 KCP protein relative to binding of the two proteins in the absence of the antagonist;
(c) the CLEC4G antagonist results in a decrease in the binding of CLEC4G and the HHV8 KCP protein relative to binding of the two proteins in the absence of the antagonist;
(d) the FLRT1 antagonist results in a decrease in the binding of FLRT1 and the HHV8 KCP protein relative to binding of the two proteins in the absence of the antagonist;
(e) the FLRT2 antagonist results in a decrease in the binding of FLRT2 and the HHV8 KCP protein relative to binding of the two proteins in the absence of the antagonist; or
(f) the FLRT3 antagonist results in a decrease in the binding of FLRT3 and the HHV8 KCP protein relative to binding of the two proteins in the absence of the antagonist.

171. The method of any one of claims 168-170, wherein the KLRAP1 antagonist, LILRB1 antagonist, CLEC4G antagonist, FLRT1 antagonist, FLRT2 antagonist, or FLRT3 antagonist reduces the extent and/or severity of HHV8 infection of the individual relative to infection in the absence of the KLRAP1 antagonist, LILRB1 antagonist, CLEC4G antagonist, FLRT1 antagonist, FLRT2 antagonist, or FLRT3 antagonist, respectively.

172. The method of any one of claims 168-171, wherein the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

173. The method of claim 172, wherein the inhibitory nucleic acid is an ASO or a siRNA.

174. The method of claim 172, wherein the KLRAP1 antagonist, LILRB1 antagonist, CLEC4G antagonist, FLRT1 antagonist, FLRT2 antagonist, or FLRT3 antagonist is a peptide.

175. The method of claim 172, wherein the KLRAP1 antagonist, LILRB1 antagonist, CLEC4G antagonist, FLRT1 antagonist, FLRT2 antagonist, or FLRT3 antagonist is an antibody or antigen-binding fragment thereof.

176. The method of claim 175, wherein:
(a) the antibody or antigen-binding fragment thereof binds the HHV8 K14 protein and inhibits its binding to KLRAP1; or
(b) the antibody or antigen-binding fragment thereof binds the HHV8 KCP protein and inhibits its binding to LILRB1, CLEC4G, FLRT1, FLRT2, and/or FLRT3.

177. The method of claim 175, wherein the antibody or antigen-binding fragment thereof binds KLRAP1, LILRB1, CLEC4G, FLRT1, FLRT2, or FLRT3.

178. The method of claim 177, wherein:
(a) the antibody or antigen-binding fragment thereof inhibits the binding of KLRAP1 to the HHV8 K14 protein; or
(b) the antibody or antigen-binding fragment thereof inhibits the binding of LILRB1, CLEC4G, FLRT1, FLRT2, or FLRT3 to the HHV8 KCP protein.

179. The method of any one of claims 172 and 175-178, wherein the antigen-binding fragment is a bis-Fab, an Fv, a Fab, a Fab′-SH, a F(ab′)₂, a diabody, a linear antibody, an scFv, an scFab, a VH domain, or a VHH domain.

180. The method of any one of claims 168-179, wherein the individual has Kaposi's sarcoma, primary effusion lymphoma, HHV8-associated multicentric Castleman's disease, or KSHV inflammatory cytokine syndrome.

181. The method of any one of claims 168-180, wherein the individual is a human.

182. A KLRAP1 antagonist, a CLEC4G antagonist, a FLRT1 antagonist, a FLRT2 antagonist, or a FLRT3 antagonist for use as a medicament.

183. A KLRAP1 antagonist, LILRB1 antagonist, CLEC4G antagonist, FLRT1 antagonist, FLRT2 antagonist, or FLRT3 antagonist for use as a medicament, wherein the medicament is for treating an HHV8 infection.

184. A KLRAP1 antagonist, LILRB1 antagonist, CLEC4G antagonist, FLRT1 antagonist, FLRT2 antagonist, or FLRT3 antagonist for use as a medicament, wherein the medicament is for treating Kaposi's sarcoma, primary effusion lymphoma, HHV8-associated multicentric Castleman's disease, or KSHV inflammatory cytokine syndrome.

185. The KLRAP1 antagonist, LILRB1 antagonist, CLEC4G antagonist, FLRT1 antagonist, FLRT2 antagonist, or FLRT3 antagonist for use of claim 183 or 184, wherein:
(a) the KLRAP1 antagonist results in a decrease in the binding of KLRAP1 and the HHV8 K14 protein relative to binding of the two proteins in the absence of the antagonist;
(b) the LILRB1 antagonist results in a decrease in the binding of LILRB1 and the HHV8 KCP protein relative to binding of the two proteins in the absence of the antagonist;
(c) the CLEC4G antagonist results in a decrease in the binding of CLEC4G and the HHV8 KCP protein relative to binding of the two proteins in the absence of the antagonist;
(d) the FLRT1 antagonist results in a decrease in the binding of FLRT1 and the HHV8 KCP protein relative to binding of the two proteins in the absence of the antagonist;
(e) the FLRT2 antagonist results in a decrease in the binding of FLRT2 and the HHV8 KCP protein relative to binding of the two proteins in the absence of the antagonist; or
(f) the FLRT3 antagonist results in a decrease in the binding of FLRT3 and the HHV8 KCP protein relative to binding of the two proteins in the absence of the antagonist.

186. The KLRAP1 antagonist, LILRB1 antagonist, CLEC4G antagonist, FLRT1 antagonist, FLRT2 antagonist, or FLRT3 antagonist for use of any one of claims 183-185, wherein the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

187. The KLRAP1 antagonist, LILRB1 antagonist, CLEC4G antagonist, FLRT1 antagonist, FLRT2 antagonist, or FLRT3 antagonist for use of claim 186, wherein the inhibitory nucleic acid is an ASO or a siRNA.

188. The KLRAP1 antagonist, LILRB1 antagonist, CLEC4G antagonist, FLRT1 antagonist, FLRT2 antagonist, or FLRT3 antagonist for use of claim 186, wherein the KLRAP1 antagonist, LILRB1 antagonist, CLEC4G antagonist, FLRT1 antagonist, FLRT2 antagonist, or FLRT3 antagonist is a peptide.

189. The KLRAP1 antagonist, LILRB1 antagonist, CLEC4G antagonist, FLRT1 antagonist, FLRT2 antagonist, or FLRT3 antagonist for use of claim 186, wherein the KLRAP1 antagonist, LILRB1 antagonist, CLEC4G antagonist, FLRT1 antagonist, FLRT2 antagonist, or FLRT3 antagonist is an antibody or antigen-binding fragment thereof.

190. The KLRAP1 antagonist, LILRB1 antagonist, CLEC4G antagonist, FLRT1 antagonist, FLRT2 antagonist, or FLRT3 antagonist for use of claim 189, wherein:
(a) the antibody or antigen-binding fragment thereof binds the HHV8 K14 protein and inhibits its binding to KLRAP1; or
(b) the antibody or antigen-binding fragment thereof binds the HHV8 KCP protein and inhibits its binding to LILRB1, CLEC4G, FLRT1, FLRT2, and/or FLRT3.

191. The KLRAP1 antagonist, LILRB1 antagonist, CLEC4G antagonist, FLRT1 antagonist, FLRT2 antagonist, or FLRT3 antagonist for use of claim 189, wherein the antibody or antigen-binding fragment thereof binds KLRAP1, LILRB1, CLEC4G, FLRT1, FLRT2, or FLRT3.

192. The KLRAP1 antagonist, LILRB1 antagonist, CLEC4G antagonist, FLRT1 antagonist, FLRT2 antagonist, or FLRT3 antagonist for use of claim 191, wherein:
(a) the antibody or antigen-binding fragment thereof inhibits the binding of KLRAP1 to the HHV8 K14 protein; or
(b) the antibody or antigen-binding fragment thereof inhibits the binding of LILRB1, CLEC4G, FLRT1, FLRT2, or FLRT3 to the HHV8 KCP protein.

193. The KLRAP1 antagonist, LILRB1 antagonist, CLEC4G antagonist, FLRT1 antagonist, FLRT2 antagonist, or FLRT3 antagonist for use of any one of claims 186 and 189-192, wherein the antigen-binding fragment is a bis-Fab, an Fv, a Fab, a Fab′-SH, a F(ab′)₂, a diabody, a linear antibody, an scFv, an scFab, a VH domain, or a VHH domain.

194. Use of a KLRAP1 antagonist, a LILRB1 antagonist, a CLEC4G antagonist, a FLRT1 antagonist, a FLRT2 antagonist, or a FLRT3 antagonist in the manufacture of a medicament for treatment of an HHV8 infection.

195. Use of a KLRAP1 antagonist, a LILRB1 antagonist, a CLEC4G antagonist, a FLRT1 antagonist, a FLRT2 antagonist, or a FLRT3 antagonist in the manufacture of a medicament for treatment of Kaposi's sarcoma, primary effusion lymphoma, HHV8-associated multicentric Castleman's disease, or KSHV inflammatory cytokine syndrome.

196. Use of a KLRAP1 antagonist, a LILRB1 antagonist, a CLEC4G antagonist, a FLRT1 antagonist, a FLRT2 antagonist, or a FLRT3 antagonist in the manufacture of a medicament for reducing or preventing infection of a cell by HHV8.

197. The use of any one of claims 194-196, wherein:
(a) the KLRAP1 antagonist results in a decrease in the binding of KLRAP1 and the HHV8 K14 protein relative to binding of the two proteins in the absence of the antagonist;
(b) the LILRB1 antagonist results in a decrease in the binding of LILRB1 and the HHV8 KCP protein relative to binding of the two proteins in the absence of the antagonist;
(c) the CLEC4G antagonist results in a decrease in the binding of CLEC4G and the HHV8 KCP protein relative to binding of the two proteins in the absence of the antagonist;
(d) the FLRT1 antagonist results in a decrease in the binding of FLRT1 and the HHV8 KCP protein relative to binding of the two proteins in the absence of the antagonist;
(e) the FLRT2 antagonist results in a decrease in the binding of FLRT2 and the HHV8 KCP protein relative to binding of the two proteins in the absence of the antagonist; or
(f) the FLRT3 antagonist results in a decrease in the binding of FLRT3 and the HHV8 KCP protein relative to binding of the two proteins in the absence of the antagonist.

198. The use of any one of claims 194-197, wherein the KLRAP1 antagonist, LILRB1 antagonist, CLEC4G antagonist, FLRT1 antagonist, FLRT2 antagonist, or FLRT3 antagonist is a small molecule, an antibody or antigen-binding fragment thereof, a peptide, a mimic, or an inhibitory nucleic acid.

199. The use of claim 198, wherein the inhibitory nucleic acid is an ASO or a siRNA.

200. The use of claim 198, wherein the KLRAP1 antagonist, LILRB1 antagonist, CLEC4G antagonist, FLRT1 antagonist, FLRT2 antagonist, or FLRT3 antagonist is a peptide.

201. The use of claim 198, wherein the KLRAP1 antagonist, LILRB1 antagonist, CLEC4G antagonist, FLRT1 antagonist, FLRT2 antagonist, or FLRT3 antagonist is an antibody or antigen-binding fragment thereof.

202. The use of claim 201, wherein:
(a) the antibody or antigen-binding fragment thereof binds the HHV8 K14 protein and inhibits its binding to KLRAP1; or
(b) the antibody or antigen-binding fragment thereof binds the HHV8 KCP protein and inhibits its binding to LILRB1, CLEC4G, FLRT1, FLRT2, and/or FLRT3.

203. The use of claim 201, wherein the antibody or antigen-binding fragment thereof binds KLRAP1, LILRB1, CLEC4G, FLRT1, FLRT2, or FLRT3.

204. The use of claim 203, wherein:
(a) the antibody or antigen-binding fragment thereof inhibits the binding of KLRAP1 to the HHV8 K14 protein; or
(b) the antibody or antigen-binding fragment thereof inhibits the binding of LILRB1, CLEC4G, FLRT1, FLRT2, or FLRT3 to the HHV8 KCP protein.

205. The use of any one of claims 198 and 201-204, wherein the antigen-binding fragment is a bis-Fab, an Fv, a Fab, a Fab′-SH, a F(ab′)₂, a diabody, a linear antibody, an scFv, an scFab, a VH domain, or a VHH domain.

206. A method of identifying a modulator of the interaction between the HCMV UL6 protein and MERTK or VEGFR2, the method comprising:
(a) providing a candidate modulator;
(b) contacting the HCMV UL6 protein with MERTK or VEGFR2 in the presence or absence of the candidate modulator under conditions permitting the binding of the HCMV UL6 protein to MERTK or VEGFR2; and
(c) measuring the binding of the HCMV UL6 protein to MERTK or VEGFR2, wherein an increase or decrease in binding in the presence of the candidate modulator relative to binding in the absence of the candidate modulator identifies the candidate modulator as a modulator of the interaction between the HCMV UL6 protein and MERTK or VEGFR2.

207. A method of identifying a modulator of a downstream activity of the HCMV UL6 protein, the method comprising:
(a) providing a candidate modulator;
(b) contacting the HCMV UL6 protein with MERTK or VEGFR2 in the presence or absence of the candidate modulator under conditions permitting the binding of the HCMV UL6 protein to MERTK or VEGFR2; and
(c) measuring a downstream activity of the HCMV UL6 protein, wherein a change in the downstream activity in the presence of the candidate modulator relative to the downstream activity in the absence of the candidate modulator identifies the candidate modulator as a modulator of the downstream activity of the HCMV UL6 protein.

208. A method of identifying a modulator of a downstream activity of MERTK or VEGFR2, the method comprising:
(a) providing a candidate modulator;
(b) contacting MERTK or VEGFR2 with the HCMV UL6 protein in the presence or absence of the candidate modulator under conditions permitting the binding of MERTK or VEGFR2 to the HCMV UL6 protein; and
(c) measuring a downstream activity of MERTK or VEGFR2, wherein a change in the downstream activity in the presence of the candidate modulator relative to the downstream activity in the absence of the candidate modulator identifies the candidate modulator as a modulator of the downstream activity of MERTK or VEGFR2.

209. The method of claim 206, wherein the increase or decrease in binding is at least 70%, as measured by a surface plasmon resonance (SPR) assay, a BLI assay, or an enzyme-linked immunosorbent assay (ELISA).

210. The method of claim 207 or 208, wherein the modulator is an inhibitor of the downstream activity of the HCMV UL6 protein or MERTK or VEGFR2.

211. The method of claim 207 or 208, wherein the modulator is an activator of the downstream activity of the HCMV UL6 protein or MERTK or VEGFR2.

212. The method of claim 207 or 208, wherein the change in the downstream activity is a decrease in the amount, strength, or duration of the downstream activity.

213. The method of claim 207 or 208, wherein the change in the downstream activity is an increase in the amount, strength, or duration of the downstream activity.

214. The method of any one of claims 207-210 and 212, wherein the downstream activity is infection of a cell by HCMV.

215. The method of claim 214, wherein infection is decreased in the presence of the modulator.

216. The method of claim 215, wherein infection is decreased by at least 40%, as measured in a viral infection assay or a viral entry assay.

217. The method of any one of claims 207-210 and 212, wherein the downstream activity is angiogenesis.

218. The method of claim 217, wherein angiogenesis is decreased by at least 40%, as measured in a tube formation assay.

219. The method of any one of claims 207-210 and 212, wherein the modulator is an inhibitor of a downstream activity of the HCMV UL6 protein or MERTK, and the downstream activity is phosphorylation of MERTK.

220. The method of claim 219, wherein phosphorylation of MERTK is decreased by at least 40%, as measured using a Western blot or ELISA.

221. The method any one of claims 206-220, wherein the modulator is a small molecule, an antibody or antigen-binding fragment thereof, a peptide, a mimic, an antisense oligonucleotide, or a small interfering RNA (siRNA).

222. The method of claim 221, wherein the antigen-binding fragment is a bis-Fab, an Fv, a Fab, a Fab′-SH, a F(ab′)₂, a diabody, a linear antibody, an scFv, an ScFab, a VH domain, or a VHH domain.

223. The method of claim 221 or 222, wherein the antibody or antigen-binding fragment thereof binds the HCMV UL6 protein.

224. The method of claim 221 or 222, wherein the antibody or antigen-binding fragment thereof binds MERTK or VEGFR2.
